(19) United States
(12) Patent Application Publication
(10) Pub. No.: US 2005/0070700 A1
(43) Pub. Date: Mar. 31, 2005

(54) Equine arteritis virus vaccine

(76) Inventor: Matthias Giese, Heidelberg (DE)

Correspondence Address: Anthony P. Bottino, Bottino LLC, 49 Woodland Road, Bedford Hills, NY, 10507, US

(21) Appl. No.: 10/675,444

(22) Filed: Sep. 30, 2003

(51) Int. Cl. A61K 39/12; C12Q 1/70; C12N 15/00; C12N 15/09; C12N 15/70; C12N 15/74; C12N 15/63; A61K 39/193; C07H 21/04

(52) U.S. Cl. 536/23.72; 435/320.1; 424/218.1; 435/5

(57) ABSTRACT

Disclosed are vaccine compositions comprising open reading frame (ORF) 2, ORF 5 and ORF 7 nucleic acid of EAV, and vectors comprising said ORFs. Also disclosed are methods of using the ORFs and vectors in the manufacture of a medicament for the prevention and treatment of EAV infections.

### 1. TECHNICAL FIELD

The invention belongs to the field of animal health and in equine arteritis virus (EAV). The invention provides vaccine compositions comprising open reading frame (ORF) 2, ORF 5 and ORF 7 nucleic acid of EAV, nucleic acid said ORF2, ORF 5 and ORF 7 and vectors comprising said ORFs. The invention further relates to the use of said ORFs and vectors in the manufacture of a medicament for the prevention and treatment of EAV infections.

### 2. BACKGROUND OF THE INVENTION

Equine arteritis is a contagious disease of horses and is spread via respiratory or reproductive tract and caused by equine arteritis virus (EAV) which is a member of the Arteriviridae family, that includes lactate dehydrogenase-elevating virus (LDV), porcine reproductive and respiratory syndrome virus (PRRSV), and simian haemorrhagic fever virus (SHFV).

EAV is well investigated and its biological and biophysical properties together with the data on viral pathogenesis and cell virus interactions had been documented in over 200 scientific reports (enclosure 1). The genomic organization and transcriptional strategy of arteriviruses are shown in FIG. 1. The EAV virions are 60-65 nm in diameter and possess a 49S RNA genome that is a single-stranded, nonsegmented, capped and polyadenylated message-sense RNA (12687 nucleotides; den Boon, et al. 1991; GenBank accession number: X53459). The EAV genome is infectious and contains at least eight open reading frames (ORF) 1a, 1b, 2, 3, 4, 5, 6, and 7 (SEQ ID No. 10). The two largest viral ORFs (ORF 1a and ORF 1b) encode the viral replicase (den Boon, et al. 1991) and are located at the 5′-end of the viral genome between the nucleotide positions 1 and 9807. The ORFs 2 to 7 are overlapping and located at the 3′-end of EAV genome. These ORFs encode four known structural viral proteins and two proteins of unknown function (gene products of ORFs 3 and 4). The EAV virion consists of a phosphorylated nucleocapsid protein (N, 14 kDa, gene product of ORF 7), a N-glycosylated major membrane protein (G_{L}, 30-44 kDa, gene product of ORF 5), a N-glycosylated minor membrane protein (G_{S}, 25 kDa, gene product of ORF 2), and an unglycosylated membrane protein (M, 17 ka, gene product of ORF 6). The EAV infection is maintained in horse population, because chronic carrier animals shed EAV in their semen and transmit it venereally to mares during the mating season. PRRSV is also characterized by reproductive failure e.g. late-term abortions in sows and causing respiratory illness and mortality in young pigs.

The analysis of the genetic stability of EAV during horizontal and vertical transmission in an outbreak of equine viral arteritis revealed that the carrier stallion is the source of genetic diversity of EAV (Balasuriya et al., 1999). It is known that the infected carrier stallion is the critical natural reservoir of EAV. The outbreak of an EAV infection can be initiated by the horizontal aerosol transmission of specific viral variants that occur in the semen of carrier stallions. However, Patton and co-workers show that not only the carrier stallion is the critical natural reservoir of EAV, but also genetic diversity of the virus is generated in the course of persistent infection of carrier stallions (Patton et al., 1999).

Consequently, PRRSV and EAV cause economically important infectious diseases in swine and horse farms worldwide. The development of an efficient vaccine is of particular importance, since it focuses attention on the prevention of the diseases. In the art, there was a long lasting need for an effective EAV vaccine capable of preventing or curing an EAV-associated disease. Therefore, the technical problem underlying this invention was to provide such a vaccine capable of preventing or curing an EAV-associated disease.

### BRIEF SUMMARY OF THE INVENTION

It is therefore an object of the invention to provide a vaccine composition protective against equine arterivirius (EAV).

It is also an object of the invention to provide for methods for prophylaxis or treatment of EAV infection in a horse using such vaccine compositions.

### FIGURES

FIG. 1

Schematic diagram of the genomic organisation and transcriptional strategy of the family Arteriviridae.

FIG. 2:

Schematic diagram of the strategy used for molecular cloning of neutralizing domain of equine arteritis virus (EAV).

FIG. 3:

The results of neutralization tests obtained by the analysis of the sera of the individual Balb/c mice that were inoculated in two independent experiments (A and B) with the DNA of recombinant plasmid pCR3.1-EAV-O5-BX-C14 harboring and expressing ORF 5 of equine arteritis virus (EAV).

FIG. 4:

The results of neutralization test obtained by the analysis of the sera of the individual Balb/c mice that were inoculated with the DNA of recombinant plasmids pCR3.1-EAV-O5-BX-C14 and pCR3.1-EAV-O7-BX-C3 harboring and expressing ORFs 5 and 7 of equine arteritis virus (EAV).

FIG. 5:

The results of neutralization test obtained by the analysis of the sera of the individual Balb/c mice that were inoculated with the DNA of recombinant plasmids pDP-EAV-O5-BsS-C2 and pDP-EAV-O7-BsS-C1 harboring and expressing ORFs 5 and 7 of equine arteritis virus (EAV).

FIG. 6:

The results of neutralization test obtained by the analysis of the sera of the individual Balb/c mice that were inoculated with the DNA of recombinant plasmids pCR3.1-EAV-O5-BX-C14 and pCR3.1-EAV-O6-BE-C4 harboring and expressing ORFs 5 and 6 of equine arteritis virus (EAV).

FIG. 7:

The results of neutralization test obtained by the analysis of the sera of the individual Balb/c mice that were inoculated with the DNA of recombinant plasmid pCR3.1-EAV-O4-BX-C3 harboring and expressing ORF 4 of equine arteritis virus (EAV). The individual animals are indicated with number 1 to 10.

FIG. 8:

The results of neutralization test obtained by the analysis of the sera of the individual Balb/c mice that were inoculated with the DNA of recombinant plasmid pCR3.1-EAV-O4-BE-C3 harboring and expressing ORF 4 of equine arteritis virus (EAV). The individual animals are indicated with number 1 to 10.

FIG. 9:

The results of neutralization tests obtained by the analysis of the sera of the individual Balb/c mice that were inoculated with the DNA of recombinant plasmid pCR3.1-EAV-O5-del-121 harboring and expressing the N-terminal hydrophilic ectodomain of G_{L} envelope glycoprotein (amino acid residue 1-121 of ORF 5) of equine arteritis virus (EAV).

FIG. 10:

The results of neutralization test obtained by the analysis of the sera of the individual Balb/c mice that were inoculated with the DNA of recombinant plasmids pCR3.1-EAV-O₂-BX-C5, pCR3.1-EAV-O5-BX-C14, and pCR3.1-EAV-O6-BE-C4 harboring and expressing ORFs 2 (small glycoprotein), 5 (large envelope glycoprotein), and 6 (membrane protein), of equine arteritis virus (EAV).

FIG. 11:

The results of neutralization test obtained by the analysis of the sera of the individual Balb/c mice that were inoculated with the DNA of recombinant plasmids pCR3.1-EAV-O₂-BX-C5 and pCR3.1-EAV-O4-BX-C3 harboring and expressing ORFs 2 and 4 of equine arteritis virus (EAV).

FIG. 12:

An example of the results obtained by enzyme linked immunosorbent assay (ELISA) for the detection of EAV specific antibodies.

FIG. 13: Establishing a lymphoproliferation assay for detection of cellular immune response

FIG. 14 (FIG. 14-14b) Determination of the maximum cellular ⁵¹Cr-uptake

FIG. 15 (FIG. 15a-15e) Calculated specific lysis after the 2^{nd} and 3^{rd} booster immunization

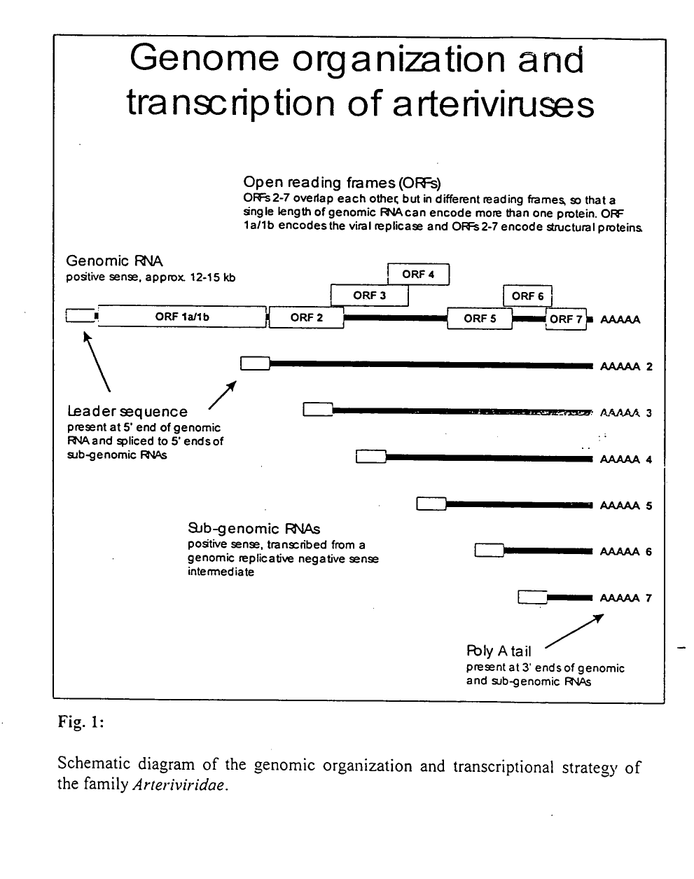

### DETAILED DESCRIPTION OF THE INVENTION

Definitions of Terms Used in the Description:

Before the embodiments of the present invention it must be noted that as used herein and in the appended claims, the singular forms “a”, “an”, and “the” include plural reference unless the context clearly dictates otherwise. Thus, for example, reference to “a nucleic acid molecule” includes a plurality of such nucleic acid molecules, reference to the “vector” is a reference to one or more vectors and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies as reported in the publications which might be used in connection with the invention. Nothing herein is to be construed as an admission that the invention is not entitled to antedate such disclosure by virtue of prior invention.

The term “EAV” as used herein refers to all viruses belonging to species equine arteritis virus within the family Arteriviridae.

A “fragment” according to the invention is any subunit of a DNA molecule (e.g. part of an open reading frame (ORF)) of a longer DNA molecule (e.g. an entire ORF) EAV according to the invention, i.e. any subset, characterized in that it is encoded by a shorter nucleic acid molecule than disclosed which can still be transcribed into RNA. “Fragment” also relates to subsets of proteins, i.e. smaller proteins encoded by said DNA fragments. The expression is to be understood depending upon the context in which it is used.

A “functional variant” of the DNA molecule according to the invention or protein encoded thereby is a DNA molecule or protein which possesses a biological activity (either functional or structural) that is substantially similar to the DNA molecule or protein according to the invention. The term “functional variant” also includes “a fragment”, “a functional variant”, “variant based on the degenerative nucleic acid code” or “chemical derivative”. Such a “functional variant” e.g. may carry one or several nucleic acid exchanges, deletions or insertions. Said exchanges, deletions or insertions may account for 10% of the entire sequence. Said functional variant at least partially retains its biological activity, e.g. function as an infectious clone or a vaccine strain, or even exhibits improved biological activity.

A “variant based on the degenerative nature of the genetic code” is a variant resulting from the fact that a certain amino acid may be encoded by several different nucleotide tripletts. Said variant at least partially retains its biological activity, or even exhibits improved biological activity.

According to the invention, “mutation” means the replacement of a nucleotide by another (e.g. C for a T) a so-called “substitution” or any other mutation such as “deletion” or “insertion”. “Deletion” means the removal of one or several nucleotides or amino acids.

A “fusion molecule” may be the DNA molecule or protein according to the invention fused to e.g. a reporter such as a radiolabel, a chemical molecule such as a fluorescent label or any other molecule known in the art.

As used herein, a “chemical derivative” according to the invention is a DNA molecule or protein according to the invention chemically modified or containing additional chemical moieties not normally being part of the molecule. Such moieties may improve the molecule's solubility, absorption, biological half life etc.

A molecule is “substantially similar” to another molecule if both molecules have substantially similar nucleotide sequences or biological activity. Thus, provided that two molecules possess a similar activity, they are considered variants as that term is used herein if the nucleotide sequence is not identical, and two molecules which have a similar nucleotide sequence are considered variants as that term is used herein even if their biological activity is not identical.

The terms “vaccine” and “vaccine composition” are used interchangeably.

The term “vaccine” as used herein refers to a pharmaceutical composition comprising at least one immunologically active component that induces an immunological response in an animal and possibly but not necessarily one or more additional components that enhance the immunological activity of said active component. A vaccine may additionally comprise further components typical to pharmaceutical compostions. The immunologically active component of a vaccine may comprise complete virus particles in either their original form or as attenuated particles in a so called modified live vaccine (MLV) or particles inactivated by appropriate methods in a so called killed vaccine (KV). In another form the immunologically active component of a vaccine may comprise appropriate elements of said organisms (subunit vaccines) whereby these elements are generated either by destroying the whole particle or the growth cultures containing such particles and optionally subsequent purification steps yielding the desired structure(s), or by synthetic processes including an appropriate manipulation by use of a suitable system based on, for example, bacteria, insects, mammalian or other species plus optionally subsequent isolation and purification procedures, or by induction of said synthetic processes in the animal needing a vaccine by direct incorporation of genetic material using suitable pharmaceutical compositions (polynucleotide vaccination). A vaccine may comprise one or simultaneously more than one of the elements described above.

The term “vaccine” as understood herein is a vaccine for veterinary use comprising antigenic substances and is administered for the purpose of inducing a specific and active immunity against a disease provoked by EAV. The EAV vaccine according to the invention confers active immunity that may be transferred passively via maternal antibodies against the immunogens it contains and sometimes also against antigenically related organisms.

Additional components to enhance the immune response are constituents commonly referred to as adjuvants, like e.g. aluminiumhydroxide, mineral or other oils or ancillary molecules added to the vaccine or generated by the body after the respective induction by such additional components, like but not restricted to interferons, interleukins or growth factors.

A “vaccine composition” essentially consists of one or more ingredients capable of modifying physiological e.g. immunological functions of the organism it is administered to, or of organisms living in or on the organism. The term includes, but is not restricted to antibiotics or antiparasitics, as well as other constituents commonly used to achieve certain other objectives like, but not limited to, processing traits, sterility, stability, feasibility to administer the composition via enteral or parenteral routes such as oral, intranasal, intravenous, intramuscular, subcutaneous, intradermal or other suitable route, tolerance after administration, controlled release properties.

Disclosure of the Invention

The solution to the above technical problem is achieved by the description and the embodiments characterized in the claims.

The long lasting need in the art for a vaccine capable of preventing or curing an EAV-associated disease has been overcome by the present inventors who provided a such a nucleic acid-based prophylactic or therapeutic vaccine for EAV-associated diseases. Said vaccine is described in more details infra.

Surprisingly, the nucleic acid-based vaccine according to the invention for the first time in the art is capable of not only generating a humoral (antibody-based) response (demonstrated in an exemplary manner in e.g. example 1), but also a cellular immune response in horses. Only this cellular immune response, as exemplified in examples 2 to 5, is protective against horizontal and vertical EAV transmission in horses. More surprisingly, quite contrary to what was expected by the artisan (Barry and Johnston, 1997) the vaccine according to the invention is protective against infection not only in young horses, but also in horses of all ages (as exemplified by data, see table 8 and 10). Thus, the vaccines according to the invention as described infra are capable of inducing a cellular immune response in horses and are protective against horizontal and vertical EAV transmission in horses of different ages.

In a first important embodiment, the invention relates to a vaccine composition which is protective against equine arterivirus (EAV) infections in horses and induces a cellular immune response, comprising a open reading frame nucleic acid (ORF) 2, ORF 5 and/or ORF7 of EAV.

The invention also relates to vaccine compositions wherein said ORF 2, ORF5, and/or ORF7 are fragments, functional variants or carry mutations as defined supra.

To prepare such nucleic acids, the artisan may follow the examples of the present invention and apply also standard molecular biology methods which can be found e.g. in Sambrook et al. (1989) Molecular Cloning: A Laboratory Manual, 2nd ed., Cold Spring Harbor Laboratory Press, Cold Spring Harbor, N.Y. and Bertram, S. and Gassen, H. G. Gentechnische Methoden, G. Fischer Verlag, Stuttgart, N.Y., 1991).

The invention further relates to a vaccine composition according to the invention, wherein said vaccine composition comprises a nucleic acid ORF 2, ORF 5 and ORF7 of EAV. Surprisingly, contrary to the opinion in the art, a vaccine comprising nucleic acid said three ORFs is particularly effective and much better than a vaccine wherein the entire cDNA for EAV is used. ORF 2 relates also to ORF 2a which is comprised by the present invention. ORF 2 encodes a small glycoprotein. ORF 5 encodes a large envelope glycoprotein. ORF 7 encodes the nucleocapsid protein. The invention also relates to vaccine compositions comprising mutated or truncated ORFs, such as ORF 5 in a deleted form (SEQ ID No. 9).

The invention further relates to a vaccine composition according to the invention as disclosed supra, wherein said vaccine composition further comprises one or several ORFs selected from the group of ORF 1a, ORF 1b, ORF 3, ORF 4, ORF 6. ORF 1a and ORF 1b encode the viral replicase. Any combination of said ORFs or all of said ORFs may be part of said vaccine. ORF 3 and ORF 4 encode proteins of yet unknown function. ORF 6 encodes an unglycosylated membrane protein. Preferably, said ORF or ORFs are as disclosed in SEQ ID No. 1 or SEQ ID No. 8, SEQ ID No. 3, SEQ ID No. 4, SEQ ID No. 6.

The invention also relates to vaccine compositions comprising mutated or truncated ORFs, such as the partial ORF1 sequence in SEQ ID No. 8.

The invention further relates to a vaccine composition according to the invention as disclosed supra, wherein said nucleic acid is cDNA.

The invention further relates to a vaccine composition according to the invention as disclosed supra, wherein said vaccine composition comprises one or several nucleic acid vectors each comprising said ORF or ORFs. One embodiment of the invention relates to such a vector comprising more than one ORF.

The invention further relates to a vaccine composition according to the invention as disclosed supra, wherein said vector(s) is/are expression vector(s).

The invention further relates to a vaccine composition according to the invention as disclosed supra, wherein said expression vector(s) comprise(s) a eukaryotic cis-acting transcription/translation sequence functionally linked to said ORF(s).

The invention further relates to a vaccine composition according to the invention as disclosed supra, wherein said expression vector is selected from the group of pCR3.1, pcDNA3.1/His A, pcDNA3.1/His B, pcDNA3.1/His C, and pDisplay (pD) Such vectors are commercially available (Invitrogen).

The invention further relates to a vaccine composition according to the invention as disclosed supra, further comprising the nucleic acid encoding interleukin 2 (IL-2) or a vector or expression vector comprising said nucleic acid encoding IL-2.

The invention further relates to a vaccine composition according to the invention as disclosed supra, further comprising pharmaceutically acceptable carrier or excipient. The invention further relates to a vaccine composition according to the invention as disclosed supra, further comprising one or several adjuvants selected from the group of Muramyl Dipeptide (MDP), Montanide 720, Poly Inosine:Cytosine (Poly I:C) or plasmid DNA comprising unmethylated cytosine, guanine dinucleotide sequence motifs (CpG).

The invention relates to a vaccine according to the invention as described supra wherein the adjuvants is any one of the compounds described in Chapter 7 (pp 141-227) of “Vaccine Design, The Subunit and Adjuvant Approach” (eds. Powell, M. F. and Newman, M. J.) Pharmaceutical Biotechnology, Volume 6, Plenum Press (New York). Examples from this compendium include Muramyl Dipeptide (MDP) and Montanide 720 as disclosed supra. Molecules such as Poly Inosine:Cytosine (Poly I:C) or “immunostimulatory nucleic acid molecules” such as plasmid DNA containing CpG motifs can also be administered as adjuvants in combination with antigens encapsulated in microparticles. An “immunostimulatory nucleic acid molecule” refers to a nucleic acid molecule, which contains an unmethylated cytosine, guanine dinucleotide sequence (i.e. “CpG DNA” or DNA containing a cytosine followed by guanosine and linked by a phosphate bond) and stimulates (e.g. has a mitogenic effect on, or induces or increases cytokine expression by) a vertebrate lymphocyte. An immunostimulatory nucleic acid molecule can be double-stranded or single-stranded. Generally, double-stranded molecules are more stable in vivo, while single-stranded molecules have increased immune activity. The instant invention is based on the finding that certain “immunostimulatory nucleic acid molecules” containing unmethylated cytosine-guanine (CpG) dinucleotides activate lymphocytes in a subject and redirect a subject's immune response from a Th2 to a Th1 (e.g. by inducing monocytic cells and other cells to produce Th1 cytokines, including IL-12, IFN-.gamma. and GM-CSF).

The invention further relates to a vaccine composition according to the invention as disclosed supra, consisting of expression vectors comprising ORF2, ORF5 and ORF7 of EAV, respectively, and optionally carrier, excipients or adjuvants and an expression vector comprising the nucleic acid encoding IL-2. Said nucleic acid is preferably equine IL-2. Preferably also, said the coding nucleic acid equine IL-2 is co-expressed on a vector as disclosed supra encoding one or several EAV ORFs.

The invention further relates to a vaccine composition according to the invention as disclosed supra, wherein ORF 2 is SEQ ID No. 2, ORF 5 is SEQ ID No. 5 or SEQ ID No. 9 and ORF 7 is SEQ ID No. 7.

Suitable for targeted delivery of the vaccine composition according to the invention are colloidal dispersion systems or liposomes. One example of a targeted delivery system for the EAV ORF nucleic acid molecules according to the invention is said colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. The preferred colloidal system of this invention is a liposome. Liposomes are artificial membrane vesicles which are useful as delivery verhicles in vitro and in vivo. These formulations may have net cationic, anionic or neutral charge characteristics are useful characteristics with in vitro, in vivo and ex vivo delivery methods. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley R and Papahadjopoulos D (1981). New generation liposomes—The engineering of an efficient vehicle for intracellular delivery of nucleic acids. Trends Biochem Sci 6, 77-80). In addition to mammalian cells, liposomes have been used for delivery of polynucleotides in plant, yeast and bacterial cells. In order for a liposome to be an efficient gene transfer vehicle of the EAV ORFs according to the invention, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino R J, and Gould-Fogerite S (1988). Liposome mediated gene transfer. BioTechniques 6, 682-690).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

The vaccine composition of the present invention may contain said recombinant vector as a naked “gene expression vector”. This means that the construct is not associated with a delivery vehicle (e.g. liposomes, colloidal particles and the like). One of the principal advantages of naked DNA vectors is the lack of a immune response stimulated by the vector itself.

The invention further relates to a vaccine composition according to the invention as disclosed supra, wherein the nucleic acid or nucleic acid vector or expression vector is encapsulated into liposomes.

Several types of liposomal preparations may be used for encapsulation, including large multilamellar vesicles, small unilamellar vesicles, neutral, anionic liposomes or simple cationic amphiphiles. Most preferred are cationic liposomes.

These synthetic gene delivery systems are described by many terms:

The cationic lipid-mediated transfection has been also called liposome-mediated-transfection, cationic liposome-mediated transfection, lipofection, cytofection, amphifection, and lipid-mediated transfection. Similarly, the complexes that are produced when cationic lipids are mixed with DNA have been referred to as cytosomes, amphisomes, liposomes, nucleolipidic particles, cationic lipid-DNA complexes, lipid-DNA complexes, DNA-lipid complexes etc. Recently, a common nomenclature was proposed: Lipoplex—replaces all of the terms for cationic lipid-nucleic acid complexes (including DNA, RNA, or synthetic oligonucleotides) and lipofection means the nucleic acid delivery mediated by lipoplexes. Any of said gene delivery system may be used according to the invention.

The positive charge on cationic lipid molecules facilitates their association with negatively charged nucleic acid as well as with membrane phospholipids (negatively charged) what is the basis for the non-specific interaction of the complex.

The specific binding to the cell is mediated by use of specific ligands for cellular receptors. Cationic Liposomes may deliver DNA either directly across the plasma membrane or via endosome compartment. Regardless of its exact entry point, much of the DNA does accumulate in the endosomes and is lost by the internal hydrolytic digestion within the endosomes. To protect the plasmid DNA several strategies may be used according to the invention. This includes the use of acidotropic, weak amines such as chloroquine, which presumably prevent DNA degradation by inhibiting endosomol acidification. But also viral fusion peptides or whole viruses may be included to disrupt endosomes or promote fusion of liposomes with endosomes and facilitate release of DNA into the cytoplasm. Such protection of the plasmid DNA is also a preferred embodiment of the invention.

The DNA concentration, the ratio of lipid reagent to DNA, the transfection time and the effect of serum are the most critical factors in each transfection.

Liposomes must be stable. In case of leakage they would lose antigen and adjuvants is premature.

Preferred is a vaccine composition according to the invention as disclosed supra comprising 0.05 μg-10 μg, preferred 0.1 μg-1 μg, most preferred 0.5 μg. Further preferred dose ranges are: low range 0.1-1.0 μg, most preferred 0.5 μg, middle range 1.1-10 μg, most preferred 15 μg, high range 11 μg-20 μg, most preferred 15 μg. The artisan knows the criteria for the ideal dose which depends on the chosen route of administration, i.e. with gene gun the vaccine is injected directly into Langerhans cells, thus very little of antigen gets lost, whereas i.m. injection requires much higher doses.

Most preferred is a vaccine composition according to the invention as disclosed supra comprising 0.5 μg of individual nucleic acid vector or preferred expression vector and preferably for 10 shots per animal, i.e. 5 μg of individual nucleic acid vector (or preferred expression vector) per vaccination, e.g. if seven nucleic acid vectors (or preferred expression vectors) are used, 35 μg per vaccination and animal (see example 2).

Also preferred is a vaccine composition according to the invention as disclosed supra comprising: low range 10-100 μg, most preferred 50 μg, middle range 101-500 μg, most preferred 200 μg, high range 501 μg-2000 μg, most preferred 1000 μg. Again, the artisan knows the criteria for the ideal dose which depends on the chosen route of administration, i.e. with gene gun the vaccine is injected directly into Langerhans cells, thus very little of antigen gets lost, whereas i.m. injection requires much higher doses.

Also most preferred is a vaccine composition according to the invention as disclosed supra comprising 50 μg of individual nucleic acid vector or preferred expression vector and preferably for 4 injections per animal, i.e. 200 μg of individual nucleic acid vector (or preferred expression vector) per vaccination, e.g. if seven nucleic acid vectors (or preferred expression vectors) are used, 1,4 mg per vaccination and animal (see example 2).

In yet another important embodiment the invention relates to a nucleic acid vector comprising nucleic acid selected from the group of ORF 1a, ORF 1b, ORF 2, ORF 3, ORF 4, ORF 5, ORF 6 and/or ORF7 of EAV. Preferably, said ORF or ORFs are as disclosed in SEQ ID No. 1 or SEQ ID No. 8, SEQ ID No. 2, SEQ ID No. 3, SEQ ID No. 4, SEQ ID No. 5, SEQ ID No. 6 and/or SEQ ID No. 7.

A preferred aspect of the invention is a nucleic acid vector according to the invention as disclosed supra, wherein said nucleic acid is DNA.

A more preferred aspect of the invention is a nucleic acid vector according to the invention as disclosed supra, wherein said nucleic acid vector is an expression vector.

Another more preferred aspect of the invention is a nucleic acid vector according to the invention as disclosed supra, wherein said expression vector comprises a eukaryotic cis-acting transcription/translation sequence functionally linked to said ORF(s).

To accomplish expression, a wide variety of vectors have been developed and are commercially available which allow inducible (e.g., LacSwitch expression vectors, Stratagene, La Jolla, Calif.) or cognate (e.g., pcDNA3 vectors, Invitrogen, Chatsworth, Calif.) expression of EAV ORF nucleotide sequences under the regulation of an artificial promoter element. Such promoter elements are often derived from CMV of SV40 viral genes, although other strong promoter elements which are active in eukaryotic cells can also be employed to induce transcription of EAV ORF nucleotide sequences. Typically, these vectors also contain an artificial polyadenylation sequence and 3′ UTR which can also be derived from exogenous viral gene sequences or from other eukaryotic genes. Furthermore, in some constructs, artificial, non-coding, spliceable introns and exons are included in the vector to enhance expression of the nucleotide sequence of interest (in this case, EAV ORF sequences). These expression systems are commonly available from commercial sources and are typified by vectors such as pCDNA3 and pZeoSV (Invitrogen, San Diego, Calif.). Innumerable commercially-available as well as custom-designed expression vectors are available from commercial sources to allow expression of any desired EAV ORF transcript in more or less any desired cell type, either constitutively or after exposure to a certain exogenous, stimulus (e.g., withdrawal of tetracycline or exposure to IPTG).

A most preferred aspect of the invention is a nucleic acid according to the invention as disclosed supra, wherein said expression vector is selected from the group of pCR3.1, pcDNA3.1/His A, pcDNA3.1/His B, pcDNA3.1/His C, and pDisplay (pD).

Another most preferred aspect of the invention is a nucleic acid according to the invention as disclosed supra, wherein said nucleic acid vector comprises a nucleic acid selected from the group of SEQ ID No. 2, SEQ ID No. 5, SEQ ID No. 9 and/or SEQ ID No. 7.

Gene gun is a ballistic system that can propel DNA-coated microparticles directly into the skin. Plasmid DNA is affixed to gold particles of about 0.45 μm. The total amount of DNA per sheet is a function of the DNA/gold ratio, i.e. 5 μg DNA/mg gold. If about 0.4 mg of gold particles are shoot into the epidermis/dermis driven by helium (helium discharge pressure of 400-450 psi), about 2 μg of DNA is inoculated into the skin.

Therefore, the fundamental difference of gene gun vs. i.m. injection is the amount of DNA required to produce an equivalent level of gene expression. This apparent difference could most likely related to the fact that i.m. or i.d. injections by needle places the DNA also into extracellular spaces, exposure of DNA to nucleases in the intestitial fluid or that the hydrostatic pressure that results from the injection of several μl of saline into a muscle rapidly drives out the DNA of protein producing cells so that only a little amount of injected DNA will be expressed in protein.

Interestingly, the gun is in general limited by the little amount of plasmid that can be delivered in one inoculation. This limit is about 2.5 μg of plasmid per shot. Exceeding this amount causes the particles to clump together; creating ‘macroparticles’ which cause increased damage to the target tissue. By contrast, milligrams of plasmid DNA can be delivered by i.m. injections using needles and the success of i.m. injection can be easily monitored by swelling of the injected muscle bundle. Qualitative differences in immunisation by the gene gun and i.m. injection can be circumvented by increasing the amount of DNA. A DNA inoculation by gene gun needs almost hairless skin. So the most animals to be vaccinated have to get shaved.

Such problems should be overcome by new generations of gene guns which also may be used according to the invention. A new device, the PowderJect® system driven by helium gas, is the first one which is able to deliver both metallic and non-metallic particles into tissues. Most preferred is the use of the PowderJect® system.

Thus, another important embodiment of the invention is a method for prophylaxis or treatment of EAV infection in a horse, comprising
(i) coating one or several DNA molecule(s) according to the invention as disclosed supra, or one or several nucleic acid vector(s) according to the invention as disclosed supra, onto carrier particles;
(ii) accelerating the coated carrier particles into epidermal cells of the horse in vivo; and
(iii) inducing a protective or therapeutic immune response in said horse upon or after exposure to EAV; and
(iv) monitoring the reduction of EAV-associated symptoms or the reduction of horizontal or vertical transmission.

Preferably, in said method as disclosed supra, the carrier particles are gold.

Any of the above-disclosed devices such as gene gun or the PowderJect® system may be used. The injection may be carried out as disclosed supra. Most preferably, said method may be carried out repeatedly. An appropriate vaccination scheme may be preferably on day 0 (basic vaccination), 2 weeks therafter, 4 weeks after the basic vaccination and 7 weeks after the basic vaccination. This is exemplified in example 2.

Also preferred vaccination schemes are:
1) only one base immunization without booster; or
2) base immunization, 1^{st} boost after 8-12 weeks; or
3) base immunization, 1^{st} boost after 8-12 weeks, 2^{nd} boost after 12 months.

Said vaccine, DNA molecule(s) according to the invention as disclosed supra, or one or several nucleic acid vector(s) according to the invention as disclosed supra may be administered by any known route of administration: preferably orally, nasally, lingually, intravenously (i.v.), intradermally (i.d.), intraepidermally (by rubbing into the skin), intranasally, vaginally, subcutaneously (s.c.), intramuscular (i.m.).

According to the invention, various vehicles for administration of the vaccine, DNA molecule(s) according to the invention as disclosed supra, or one or several nucleic acid vector(s) according to the invention as disclosed supra may be used: only plasmid ‘naked’ DNA inoculation by needle-liposomes-gold beads-biodegradable nanoparticles-virus like particles (VLP)-aerosol.

Preferred modes of administration for the vaccine, DNA molecule(s) according to the invention as disclosed supra, or one or several nucleic acid vector(s) according to the invention as disclosed supra are: injection by needle, gene gun, encapsulated in liposomes or rubbing into the skin.

Preferred doses are 0.5 μg of individual nucleic acid vector or preferred expression vector and 10 shots per animal, i.e. 5 μg of individual nucleic acid vector (or preferred expression vector) per vaccination, e.g. if seven nucleic acid vectors (or preferred expression vectors) are used, 35 μg per vaccination and animal (see example 2).

Preferred are 1 to 10, more preferably 5 or 7 or 10 shots per aninmal, most preferred are 10 shots per animal.

One preferred method of vaccination is the direct injection of plasmid DNA into skeletal muscle. Long-lasting immune responses are obtained in many cases without boost.

For the i.m. route, DNA preferably is injected by needle, whereas for the i.e. route, a gene gun preferably is used (see supra).

Yet another important embodiment of the invention is a method for prophylaxis or treatment of EAV infection in a horse, comprising
(i) injecting a vaccine composition according to the invention as disclosed supra, or one or several DNA molecule(s) according to the invention as disclosed supra, or one or several nucleic acid vector(s) according to the invention as disclosed supra into muscular cells of the horse in vivo; and
(ii) inducing a protective or therapeutic immune response in said horse upon or after exposure to EAV, and
(iii) monitoring the reduction of EAV-associated symptoms or the reduction of horizontal or vertical transmission.

Most preferably, said method may be carried out repeatedly. An appropriate vaccination scheme may be preferably on day 0 (basic vaccination), 2 weeks therafter, 4 weeks after the basic vaccination and 7 weeks after the basic vaccination. This is exemplified in example 2. Preferred doses are 50 μg of individual nucleic acid vector or preferred expression vector and 4 inoculations per animal, i.e. 200 μg of individual nucleic acid vector (or preferred expression vector) per vaccination, e.g. if seven nucleic acid vectors (or preferred expression vectors) are used, 1.4 mg per vaccination and animal (see example 2).

Also preferred is a vaccine composition according to the invention as disclosed supra comprising: low range 10-100 μg, most preferred 50 μg, middle range 101-500 μg, most preferred 200 μg, high range 501 μg-2000 μg, most preferred 1000 μg. Again, the artisan knows the criteria for the ideal dose which depends on the chosen route of administration, i.e. with gene gun the vaccine is injected directly into Langerhans cells, thus very little of antigen gets lost, whereas i.m. injection requires much higher doses.

Again, preferred vaccination schemes are:
1) only one base immunization without booster; or
2) base immunization, 1^{st} boost after 8-12 weeks; or
3) base immunization, 1^{st} boost after 8-12 weeks, 2^{nd} boost after 12 months.

Said vaccine, DNA molecule(s) according to the invention as disclosed supra, or one or several nucleic acid vector(s) according to the invention as disclosed supra may be administered by any known route of administration: preferably orally, intravenously (i.v.), intradermally, intraepidermally (by rubbing into the skin), intranasally, vaginally, subcutaneously (s.c.), intramuscular (i.m.).

Preferred modes of administration for the vaccine, DNA molecule(s) according to the invention as disclosed supra, or one or several nucleic acid vector(s) according to the invention as disclosed supra are: injection by needle, gene gun, encapsulated in liposomes or rubbing into the skin.

Preferred are 1 to 10, more preferably 3 or 4 or 5 inoculations per aninmal, most preferred are 4 inoculations per animal.

The invention further relates to the use of one or several DNA molecule(s) according to the invention as disclosed supra or one or several nucleic acid vector(s) according to the invention as disclosed supra in the manufacture of a vaccine for the prophylaxis and treatment of EAV infections. Preferred is the use of DNA molecule(s) or nucleic acid vector(s) according to the invention as disclosed supra in the manufacture of a vaccine suitable for intraepidermal, intradermal or intramuscular administration for the prophylaxis and treatment of EAV infections.

More preferred is the use of DNA molecule(s) or nucleic acid vector(s) according to the invention as disclosed supra in the manufacture of a vaccine suitable for intraepidermal or intradermal administration for the prophylaxis and treatment of EAV infections, wherein the vaccine is to be administered on day 0 (basic vaccination), 2 weeks therafter, 4 weeks after the basic vaccination and 7 weeks after the basic vaccination. This is exemplified in example 2.

More preferred is the use of DNA molecule(s) or nucleic acid vector(s) or expression vector according to the invention as disclosed supra in the manufacture of a vaccine suitable for intraepidermal or intradermal administration for the prophylaxis and treatment of EAV infections, wherein the vaccine comprises 0.5 μg of individual nucleic acid vector or preferred expression vector and preferably for 10 shots per animal, i.e. 5 μg of individual nucleic acid vector (or preferred expression vector) per vaccination, e.g. if seven nucleic acid vectors (or preferred expression vectors) are used, 35 μg per vaccination and animal (see example 2).

Preferred is the use of DNA molecule(s) or nucleic acid vector(s) or expression vector according to the invention comprising 0.05 μg-10 μg, preferred 0.1 μg-1 μg, most preferred 0.5 μg. Further preferred dose ranges are: low range 0.1-1.0 μg, most preferred 0.5 μg, middle range 1.1-10 μg, most preferred 15 μg, high range 11 μg-20 μg, most preferred 15 μg. The artisan knows the criteria for the ideal dose which depends on the chosen route of administration, i.e. with gene gun the vaccine is injected directly into Langerhans cells, thus very little of antigen gets lost, whereas i.m. injection requires much higher doses.

Again, preferred vaccination schemes are:
1) only one base immunization without booster; or
2) base immunization, 1^{st} boost after 8-12 weeks; or
3) base immunization, 1^{st} boost after 8-12 weeks, 2^{nd} boost after 12 months.

Said vaccine, DNA molecule(s) according to the invention as disclosed supra, or one or several nucleic acid vector(s) according to the invention as disclosed supra may be administered by any known route of administration: preferably orally, intravenously (i.v.), intradermally, intraepidermally (by rubbing into the skin), intranasally, vaginally, subcutaneously (s.c.), intramuscular (i.m.).

Preferred modes of administration for the vaccine, DNA molecule(s) according to the invention as disclosed supra, or one or several nucleic acid vector(s) according to the invention as disclosed supra are: injection by needle, gene gun, encapsulated in liposomes or rubbing into the skin.

Preferred are 1 to 10, more preferably 3 or 4 or 5 administrations per aninmal, most preferred are 4 administrations per animal.

More preferred is the use of DNA molecule(s) or nucleic acid vector(s) according to the invention as disclosed supra in the manufacture of a vaccine suitable for intraepidermal or intradermal administration for the prophylaxis and treatment of EAV infections, wherein the vaccine is to be administered on day 0 (basic vaccination), 2 weeks therafter, 4 weeks after the basic vaccination and 7 weeks after the basic vaccination. This is exemplified in example 2.

Preferred is the use of DNA molecule(s) or nucleic acid vector(s) or expression vector according to the invention comprising 0.05 μg-10 μg, preferred 0.1 μg-1 μg, most preferred 0.5 μg. Further preferred dose ranges are: low range 0.1-1.0 μg, most preferred 0.5 μg, middle range 1.1-10 μg, most preferred 15 μg, high range 11 μg-20 μg, most preferred 15 μg. The artisan knows the criteria for the ideal dose which depends on the chosen route of administration.

Again, preferred vaccination schemes are:
1) only one base immunization without booster; or
2) base immunization, 1^{st} boost after 8-12 weeks; or
3) base immunization, 1^{st} boost after 8-12 weeks, 2^{nd} boost after 12 months.

Said vaccine, DNA molecule(s) according to the invention as disclosed supra, or one or several nucleic acid vector(s) according to the invention as disclosed supra may be administered by any known route of administration: preferably orally, intravenously (i.v.), intradermally, intraepidermally (by rubbing into the skin), intranasally, vaginally, subcutaneously (s.c.), intramuscular (i.m.).

Preferred modes of administration for the vaccine, DNA molecule(s) according to the invention as disclosed supra, or one or several nucleic acid vector(s) according to the invention as disclosed supra are: injection by needle, gene gun, encapsulated in liposomes or rubbing into the skin.

Preferred are 1 to 10, more preferably 3 or 4 or 5 administrations per aninmal, most preferred are 4 administrations per animal.

Also more preferred is the use of DNA molecule(s) or nucleic acid vector(s) or expression vector according to the invention as disclosed supra in the manufacture of a vaccine suitable for intramuscular administration for the prophylaxis and treatment of EAV infections, wherein the vaccine comprises 50 μg of individual nucleic acid vector or preferred expression vector and preferably for 4 shots per animal, i.e. 200 μg of individual nucleic acid vector (or preferred expression vector) per vaccination, e.g. if seven nucleic acid vectors (or preferred expression vectors) are used, 1.4 mg per vaccination and animal (see example 2).

Also preferred is use of DNA molecule(s) or nucleic acid vector(s) or expression vector according to the invention as disclosed supra comprising: low range 10-100 μg, most preferred 50 μg, middle range 101-500 μg, most preferred 200 μg, high range 501 μg-2000 μg, most preferred 100 μg. Again, the artisan knows the criteria for the ideal dose which depends on the chosen route of administration, i.e. with gene gun the vaccine is injected directly into Langerhans cells, thus very little of antigen gets lost, whereas i.m. injection requires much higher doses.

Again, preferred vaccination schemes are:
1) only one base immunization without booster; or
2) base immunization, 1^{st} boost after 8-12 weeks; or
3) base immunization, 1^{st} boost after 8-12 weeks, 2^{nd} boost after 12 months.

Said vaccine, DNA molecule(s) according to the invention as disclosed supra, or one or several nucleic acid vector(s) according to the invention as disclosed supra may be administered by any known route of administration: preferably orally, intravenously (i.v.), intradermally, intraepidermally (by rubbing into the skin), intranasally, vaginally, subcutaneously (s.c.), intramuscular (i.m.).

Preferred modes of administration for the vaccine, DNA molecule(s) according to the invention as disclosed supra, or one or several nucleic acid vector(s) according to the invention as disclosed supra are: injection by needle, gene gun, encapsulated in liposomes or rubbing into the skin.

Preferred are 1 to 10, more preferably 3 or 4 or 5 administrations per aninmal, most preferred are 4 administrations per animal.

The invention also relates to the a kit of parts comprising a vaccine composition according to the invention as disclosed supra or one or several EAV ORF DNA molecule(s) according to the invention as disclosed supra or one or several nucleic acid vector(s) according to the invention as disclosed supra. Said kit is ready-to-use for vaccination of horses. Said kit may further contain, but is not limited to test tubes, other suitable containers, washing reagents and reaction buffers (which may vary in pH and magnesium concentrations), sterile water, liposomal preparations, transfection reagent such as DOTAP Liposomal (Roche) or Lipofectin, BME (Eagle's basal medium), ethanol, gold, spermidine, CaCl₂, carrier proteins and further compounds known to the skilled artisan.

The following examples serve to further illustrate the present invention; but the same should not be construed as limiting the scope of the invention disclosed herein.

### EXAMPLE 1

### Neutralization Tests

Materials and Methods

Viruses and Cells

The equine arteritis virus (EAV) used in this study was kindly provided by Professor H. Ludwig, Berlin and propagated on rabbit kidney cells (RK13, ATCC number CCL-37). The cell cultures were obtained from the American Type Culture Collection and propagated in Basal Medium Eagle (BME) supplemented with 10% fetal calf serum, 100 IE/penicillin G, 100 IE/ml streptomycin. Medium and serum were purchased from GibcoBRL (Eggenstein, Germany).

Production of EAV-Specific Antisera

Antiserum against EAV was induced in New Zealand white rabbit. The animal was inoculated subcutaneously with 0.5 ml purified EAV. Inoculation was repeated for four times. The experimental protocol is summarized in Table 1. The sensitivity of rabbit antisera was determined by western blot analysis. It was found that the rabbit antiserum raised against EAV is able to recognize viral specific protein at the dilution of about 1:2000 and higher.

Preparation of Viral RNA

Virion RNA and total infected cell RNA was prepared from EAV-infected RK13 cell cultures at 12, 24, 36, and 48 h p.i. using a guanidinium iso-thiocyanate/cesiumchloride procedure based on the method described by Gli\check{s}in et al. (1974). Infected cells or virions from clarified infected cell culture supernatants were dissolved in a 4.0 M guanidinium thiocyanate (GTC) solution. Cellular DNA in the infected cell preparation was sheared by repeatedly passing the solution through a 23-gauge needle. CsCl and sarcosyl (30% aqueous solution) were added to the GTC preparation to final concentrations of 0.15 g/ml and 3.0%, respectively. In volumes of 8 ml the preparation was transferred onto a 3 ml 5.7 M CsCl cushion and centrifuged at 29,000 rpm in a Beckman SW41 rotor for 24 h at 20° C. The supernatant was discarded and the RNA pellet was dissolved in RNase free H₂O to a final concentration of about 10 μg/ml. RNA preparations were stored at −20° C. in 80% ethanol containing 100 mM sodium acetate. As an alternative total RNA of EAV-infected cells was prepared using the RNeasy Mini Kit (Qiagen, Hilden, Germany) according to the instructions of the manufacturer.

First-Strand cDNA Synthesis

For each first-strand cDNA synthesis reaction approximately 0.5 μg of purified RNA were pelleted and dissolved in 10 μl RNase free H₂O containing 20 U RNase inhibitor (Takara Shuzo Co., Ltd., Shiga, Japan). The reaction was prepared in 20 μl volumes using enzymes and reagents from the RNA LA PCR Kit Ver.1.1 (Takara Shuzo Co., Ltd., Shiga, Japan) according to the instructions of the manufacturer. The reaction included 5 mM MgCl₂, 1 mM of each dNTP, 10 pmol of a specific reverse oligonucleotide primer, and 5 U AMV reverse transcriptase XL. The reaction was incubated in an automated temperature cycling reactor (Genius, Techne, Cambridge, UK) for 2 min at 60° C. followed by 15 min at 50° C. Then the temperature was gradually lowered to 42° C. at a speed of 1° C./min. As a final step the reaction was incubated for 2 min at 80° C. and rapidly cooled to 4° C. RNase free H₂O was added to the reaction products to obtain a final volume of 100 μl. The first-strand cDNA stocks were stored at −20° C.

Oligonucleotides and Polymerase Chain Reaction (PCR)

Specific oligonucleotides were synthesized with an Oligo 1000M DNA Synthesizer (Beckman Instruments GmbH, München, Germany). The properties of the individual oligonucleotides primers are summarized in Table 2. Polymerase chain reaction (PCR) was performed in 100 μl volumes using TaKaRa LA Taq DNA polymerase (supplied with reaction buffer, Takara Shuzo Co., Ltd., Shiga, Japan). Each reaction contained 1.5-2.5 mM MgCl₂, 12.5 nmol of each dNTP (Boehringer Mannheim Biochemica, Mannheim, Germany), 50 pmol of each oligonucleotide primer, and 1 μl of a first-strand cDNA stock solution (see above). An improved PCR protocol was developed based on a combination of commonly used hot-start and touch-down procedures. Briefly, before adding the dNTP mixture and the DNA polymerase the samples were preheated for 5 min at 94° C. and rapidly cooled to 4° C. Then dNTPs and DNA polymerase were added at 4° C. and the reaction tubes were directly transferred to a preheated temperature cycling reactor (Genius, Techne, Cambridge, UK) at 94° C. PCR reactions were incubated for 35 cycles under cycling conditions of 94° C. for 30 sec, 70-56° C. for 1 min (starting at 70° C. and decreasing by 0.4° C. per cycle), and 72° C. for 1-5 min, depending on the size of the expected PCR product. As a final step the reaction mixture was incubated for 7 min at 72° C. Reaction products were analyzed by polyacrylamide slab gel electrophoresis and ethidium bromide staining.

Molecular Cloning of Viral cDNA and Preparation of Plasmid DNA

PCR products representing EAV-specific cDNA sequences were subjected to restriction endonuclease treatment and restriction fragments were purified using preparative low melting point agarose gel electrophoresis. Specific DNA bands were extracted from the gel by a hot phenol procedure followed by gel filtration. Restricted and purified EAV cDNA was inserted into one of the following mammalian expression vectors: pCR3.1, pcDNA3.1, pcDNA3.1/His, pDisplay (Invitrogen). Vector plasmids were prepared using restriction endonucleases and purified as described above. In addition, restricted vector DNA was dephosphorylated using calf intestine phosphatase (CIP). Ligation of specific EAV cDNA fragments with expression vector DNA was performed as described previously (Rösen-Wolff et al., 1991). The resulting recombinant plasmid constructs are listed in Table 2. The specificity of the reaction products was confirmed by nucleotide sequence analysis of the insert and flanking vector regions.

Nucleotide Sequence Analysis

PCR products were treated with 1 vol phenol:chloroform (5:1) and precipitated with 3 vol 95% ethanol containing 100 mM sodium acetate. The DNA was then washed with 70% ethanol and dissolved in bidestilled water to a final concentration of 20 ng/μl. Plasmid DNA was prepared using the Qiagen tip100 Kit (Qiagen, Hilden, Germany) according to the instructions of the manufacturer. Purified DNA was adjusted in H₂O to a final concentration of 1 μg/μl. Purified DNA was automatically sequenced with a 373A “Extended” DNA sequencer using the BigDye Terminator-Taq cycle sequencing technique (Applied Biosystems GmbH, Weiterstadt, Germany). Each sequencing reaction was performed in a volume of 20 μl containing 100 ng of a PCR product or 0.5 μg of plasmid DNA, 50 pmol of the sequencing primer, and 5 μl of the BigDye Terminator reaction mixture. The cycle sequencing reaction was incubated for 28 cycles in an automated temperature cycling reactor (GeneE, Techne, Cambridge, UK) under cycling conditions of 96° C. for 30 sec and 60° C. for 4 min per cycle. The samples were prepared for electrophoresis as described by the manufacturer. The electrophoresis of the samples was carried out on a 36-well 48-cm WTR (well to read) polyacrylamide gel. The nucleotide sequences obtained from individual sequencing reactions were assembled using the Sequence Navigator software (version 2.1, Applied Biosystems GmbH, Weiterstadt, Germany). Nucleotide and amino acid sequences were compared to current GenBank, EMBL, and SwissProt database sequence entries using the BLAST service of the National Center for Biotechnology Information (National Library of Medicine, Bethesda, Md., USA). Physico-chemical properties of proteins were determined and conserved sequence motifs were identified with the PHYSCHEM and PROSITE programs included in the PC/Gene software (release 6.85, A. Bairoch, University of Geneva, Switzerland). The ClustalX program (version 1.64b) (Thompson et al., 1997) was used to generate multiple sequence alignments.

Preparation of Viral RNA and Northern Blot Analysis

Total cellular RNA was isolated at different times after infection using the guanidium/cesium chloride method as described previously (Rösen-Wolff et al., 1988, 1989; Rösen-Wolff and Darai, 1991). The northern blot analyses of these RNAs were carried out using formaldehyde agarose gel (1%) electrophoresis as described elsewhere (Rösen-Wolff et al., 1988, 1989; Rösen-Wolff and Darai, 1991).

DNA Vaccination of Animals

The immunogenic potential of the EAV translation units were investigated in vivo using BALB/c mice that were administered with DNA of the constructed expression vectors harboring and expressing the cDNA of the individual ORFs of EAV. The BALB/c mice were injected with about 100 μg/DNA diluted in 100 μl PBS. The DNA was injected subcutaniously and into the tibialis cranialis muscle (Musculus gastrocnemius) and with a 27 gauge needle. The mice were boosted 3 to 5 times with the same quantities of DNA and under the same conditions at about two week intervals. Control mice received the same amount of parental expression vectors via an identical route and frequency.

Neutralization Test

Neutralization tests were carried out by diluting EAV-specific mouse or rabbit sera with PBS (1:2 to 1:1024) in a Falcon microtiter plate. Serum dilutions (50 μl) were mixed with 100 TCID₅₀ of EAV (50 μl). The serum-virus mixture was incubated in a 5% CO₂-air atmosphere at 37° C. for 2 h. Subsequently, 5×10³ RK13 cells in suspension were added to each sample of the serum-virus mixture. After 12 h the infected cultures were overlayed with BME containing 10% FCS and 0.5% carboxymethylcellulose. Then the cultures were incubated for 3-4 days at 37° C. in a 5% CO₂-air atmosphere. Titers of infectious units were determined after staining with 1% crystal violet.

Immunoblot Analysis

Confluent monolayers of cells were harvested by scraping the cells from the culture well, petri dishes, and/or flasks after being washed three times with PBS (pH 7.2). The final cell pellet was resuspended in distilled water. Protein concentration was measured under the standard method (Bradford, 1976). Samples were dissolved in an equal volume of lysis buffer (0.01 M Tris HCl, 10% glycerol, 2% SDS, 5% β-mercaptoethanol, 0.1% (w/v) bromophenol blue, pH 8), heated for five minutes at 95° C., and subjected to SDS-PAGE according to the method of Laemmli (1970). Proteins derived from infected and transfectant cells, as well as recombinant N protein were separated by SDS-PAGE and electroblotted onto nitrocellulose filters using semi-dry electroblotting chambers (Renner, Dannstadt, Germany). Transfer efficiency was monitored by Ponceau staining (Sigma, Munich, Germany). Filters were blocked for 1 h and incubated with a 1:1000 and 1:2000 dilution of the rabbit antisera mentioned above. Alkaline phosphatase conjugated antibodies (anti rabbit or mouse Ig-AP, Boehringer Mannheim, Germany) were used to detect interaction of the rabbit or mouse antiserum with EAV protein.

Immunofluorescence Assay

Indirect Immunofluorescence assay was performed essentially as described earlier (Welzel et al., 1998). Briefly, RK13 cell lines were seeded on tissue chamber slides (Nunc Inc., Naperville, USA). The monolayer cell culture were infected with EAV at the MOI of 2 PFU/cell and 48 h after infection the cells were fixed with acetone-methanol and the slides were stored at −20° C. Fluorescein isothiocyanate (FITC)-conjugated anti-rabbit or mice IgG (F(ab′)2 fragment immunoglobulin (Boehringer, Mannheim, FRG) was used as second antibody. In addition, rhodamine B-isothiocyanate (Merck, Darmstadt, Germany) was used at a final concentration of 10 ng/μl for counterstaining of the cells, together with the second antibody.

Enzyme Linked Immunosorbent Assay (ELISA)

Polysorp F8 Microtiter Plates (Nunc, Wiesbaden, Deutschland) were coated with 50 μl EAV Protein (EAV+Host (RK13)) at a concentration of 2 μg×ml⁻¹ in PBS (+0.05% N₃Na) over night at room temperature, followed by three cycles of washing with H₂O and then postcoated with 300 μl Blocking Buffer (0.017 M Na₂B₄O₇×10 H₂O, 0.12 M Na Cl, 0.05% Tween 20, 1 mM EDTA, 5% BSA, 0.05% NaN₃) for 3 h at 28° C. followed by three cycles of washing with H₂O.

For the assay, the following reagents were successively used: rabbit anti EAV serum at a reciprocal dilution up to 16000 or mouse anti EAV serum at a reciprocal dilution up to 800. The dilutions were made in Sample buffer POD (DADE Behring, Marburg, Deutschland). After three cycles of washing with 300 μl/well, horse-radish peroxidase labeled rabbit-anti IgG second antibody or the horse-radish peroxidase labeled mouse-anti IgG second antibody (Boehringer, Mannheim, Deutschland) at a predetermined optimum dilution of 1:3000 each was added. The dilutions were made in Blocking buffer. Incubation steps were done for 1 h at 28° C., each followed by three cycles of washing with 300 μl/well Washing Buffer (DADE Behring, Marburg, Deutschland). Color was developed by adding 200 μl/well of freshly prepared Buffer/Substrate TMB and Chromogen TMB (10:1) (DADE Behring, Marburg, Deutschland). The assay was stopped after 30 min by the addition of 50 μl/well Stopping Solution POD (DADE Behring, Marburg, Deutschland) and read according to standard procedures at 450 nm on an automatic ELISA reader (MR5000, DYNATECH, Denkendorf, Deutschland).

Lymphocyte Activation Assay

A Lymphocyte activation assay for the eventual use of the cellular response of the immunized mice was established. BALB/c mouse, female, anti-mouse CD3ε (500A2, PharMingen, Cat. No. 01511D), anti-mouse CD69 (H1.2F3, PharMingen, Cat. No. 01505B),

Geys buffer (140 mM NH₄Cl, 2.7 mM KCl, 6.5 mM Na₂HPO₄, 1.5 mM KH₂PO₄, 0.7 mM CaCl₂, 0.5 mM MgCl₂), FACS buffer (PBS, 2% FCS, 0.01% NaN₃), Polystyrene round bottom tubes (Falcon, Becton Dickinson, Cat. No. 2052), and six well plate (TPP, Cat. No. 9206) were used. The mesurement of the samples was performed using FACScan (Becton Dickinson), five measurable parameters: three high performance secondary photomultiplier with bandpass filter: 530 nm for fluorescein isothiocyanate (FITC), 585 nm for phycoerythrin (PE) and 650 nm (red fluorescence), forward and side scatter.

One mouse was killed, spleenectomy was performed and spleen was in a sterile tube with 1×BME medium. Spleen was passed through a sterile homogeniser for intact spleen cell separation. The homogeniser was washed with 1×BME medium. The solution was centrifuged for 3 min with 1500 rpm. The pellet was dissolved in 1.5 ml Geys buffer. After that the solution was centrifuged for 3 min with 1500 rpm. The pellet was dissolved in 9 ml BME medium with 10% FCS. One 6 well plate was filled with 1 ml 1×BME medium with 10% FCS, in 3 wells of the plate additional 1 μg/ml anti-mouse CD3ε was added. 1.5 ml cell solution was added to each well. The 6 well plate was incubated at 37° C., 5% CO₂ for three days. Solution with activated and not activated cells were each divided in four Polystyrene round bottom tubes. The tubes were filled with FACS buffer and centrifuged for 3 min with 1500 rpm. The supernatant was discarded and the pellet was resuspended in two of four tubes with 100 μl anti-mouse CD3ε (1:100 diluted in FACS buffer) and incubated for 30 minutes at 4° C. For double fluorescence the cells were centrifuged for 3 min with 1500 rpm, the pellet was resuspended with 100 μl anti-mouse CD69 (1:100 diluted in FACS buffer) and incubated for 30 minutes at 4° C. again. The cells were centrifuged for 3 min with 1500 rpm, the pellet was resuspended in 2 ml FACS buffer, centrifuged again for 3 min with 1500 rpm, and resuspended in 500 μl FACS buffer each, vortexed and measured in the FACScan.

Computer-Assisted Sequence Analysis

Nucleotide sequences were compiled using the ABI sequence navigator software version 1.2. Nucleotide and amino acid sequences were analyzed using the PC/GENE program release 6.85 (Intelligenetics Inc. Mountain View, Calif., U.S.A.) and OMIGA program release 11.3 (Oxford Molecular Group Ltd., Oxford, UK).

Results

Establishment of a Cell Culture System for Virus Propagation, Isolation of Virus Particles, and Extraction of Viral RNA

The equine arteritis virus (EAV) used in this study was grown and propagated on rabbit kidney cells (RK-13). Virions of EAV were prepared, purified, and the viral RNA was extracted.

Generation of Specific Rabbit Antisera Against Whole Virus

In order to specifically detect viral gene products by serological assays a polyclonal hyperimmune rabbit antiserum was raised against complete EAV virion components in New Zealand white rabbits. The protocol of the immunization experiment is given in Table 1. The titer of the antiserum was found to be >1:2000 as determined by Western Blot analysis.

Generation of Rabbit Antisera Against Synthetic Polypeptides

The biophysical analysis of the nucleocapsid protein (ORF 7) of EAV indicated that a strong antigenic domain was located within the first 38 amino acids of the ORF 7 gene product. In order to investigate the immunogenic potential of the N-terminus of the viral nucleocapsid protein a synthetic polypeptide corresponding to amino acid residues 1-38 was synthesized and linked to keyhole limpet hemocyanine (KLH) and used to raise polyclonal monospecific antiserum in rabbits. The antiserum was found to detect specific EAV gene product in Western Blot assay at a dilution of 1:200.

Amplification of Viral Genes by RT-PCR and Molecular Cloning of Viral Genes in Expression Vectors

The EAV genome consists of about 12.287 nucleotides with a short 3′poly-(A) tail. For molecular cloning of the viral genome specific oligonucleotides were synthesized (Table 2) and purified viral RNA was used in 3′-RACE experiments to generate a cDNA bank from the genomic RNA including cDNA of viral mRNA transcripts. The amplified cDNA fragments include the viral open reading frames (ORFs) 2 to 7, which encode the small glycoprotein, nonstructural proteins, large glycoprotein, membrane protein, and nucleocapsid protein, respectively (Table 3). The viral genes were molecularly cloned into a suitable expression vector (pCR3.1, pDisplay, and pcDNA3.1/His A, B, C; Invitrogen). The identity of the cDNA of the individual viral ORFs was confirmed by nucleotide sequence analysis. The properties of the constructed mammalian expression vectors harboring and expressing EAV-specific cDNA of the individual viral translation units are summarized in Table 3.

As an additional goal for the development of efficient EAV DNA vaccines in future it is envisaged to construct expression vectors harboring and expressing overlapping or full-length viral cDNA. An expression library of cloned high molecular weight viral cDNA was constructed spanning the entire viral genome. Using optimized long-range RT-PCR protocols a number of overlapping viral cDNA fragments ranging from 2,000 to over 8,500 bp were generated. The identities of the RT-PCR products were confirmed by nucleotide sequence analysis. This library is essential to establish full-length cDNA clones expressing the entity of viral antigens simultaneously in in vivo and in vitro when necessary. The properties of the individual RT-PCR products are summarized in Table 4.

DNA Vaccination of Mice with Different Vector Constructs and Evaluation of the Corresponding Immune Response

DNA Vaccination of Mice with Vector Construct Expressing Viral ORFs 5 and 7

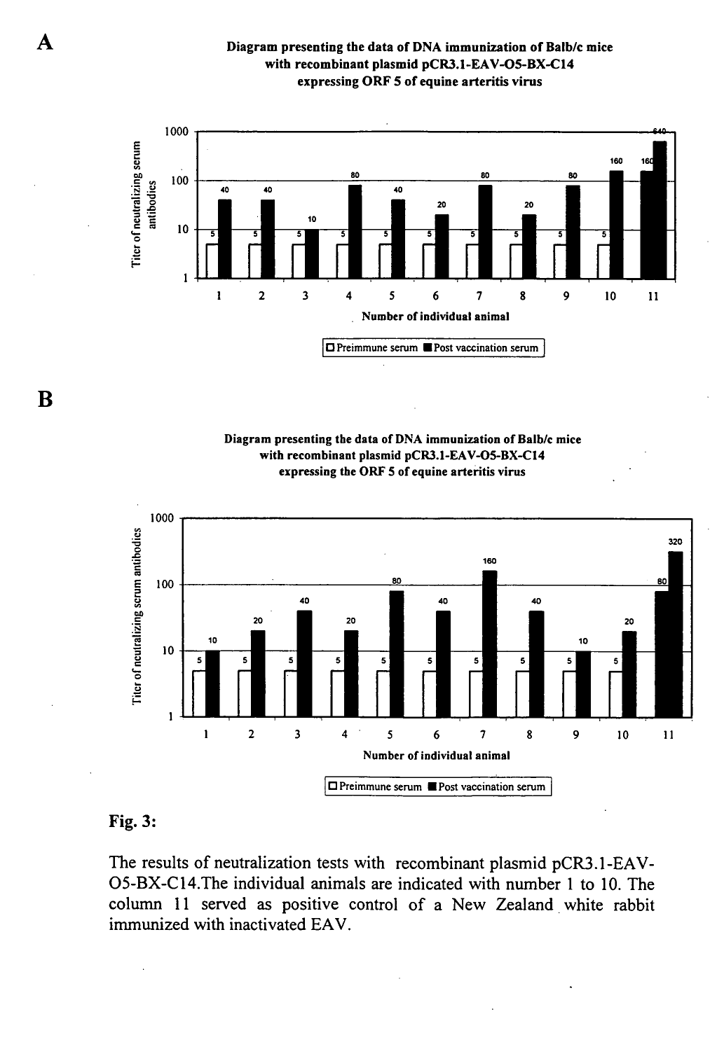

Balb/c mice were used as a model system for the evaluation of the immune responses against individual gene products of EAV raised by administration of recombinant plasmid DNA. The capability of the expression vectors harboring and expressing EAV ORF5 and ORF7 that encode the viral nucleocapsid protein and the viral major glycoprotein to induce immune response in the mouse system was investigated. The animals were inoculated subcutaneously and intramuscularly for five times with 100 μg of the particular DNA in 14-day intervals. Analysis of the individual mouse sera revealed that the administered DNAs of the vectors expressing both viral gene products are able to induce significant immune response in mice as tested by neutralization test (NT). The results of these studies are summarized in Table 5 and are shown in FIG. 3 for ORF 5. A significant immune response was detected when the animals were immunized with the recombinant plasmids pCR3.1-EAV-O7-BX-C3 and pCR3.1-EAV-O5-C14 harboring ORFs 7 and 5 coding for viral nucleocapsid protein and large envelope glycoprotein (G_{L}), respectively. As shown in Table 5 it was found that the recombinant plasmids pCR3.1-EAV-O7-BX-C3 are able to raise antibodies against EAV in mice (80% immune response) as detected by Neutralization Test. Similar results (50-70% immune response) were obtained in two independent experiments when the animals were inoculated with the recombinant plasmids pCR3.1-EAV-O5-BX-C14 (Table 5 and FIGS. 3A and B).

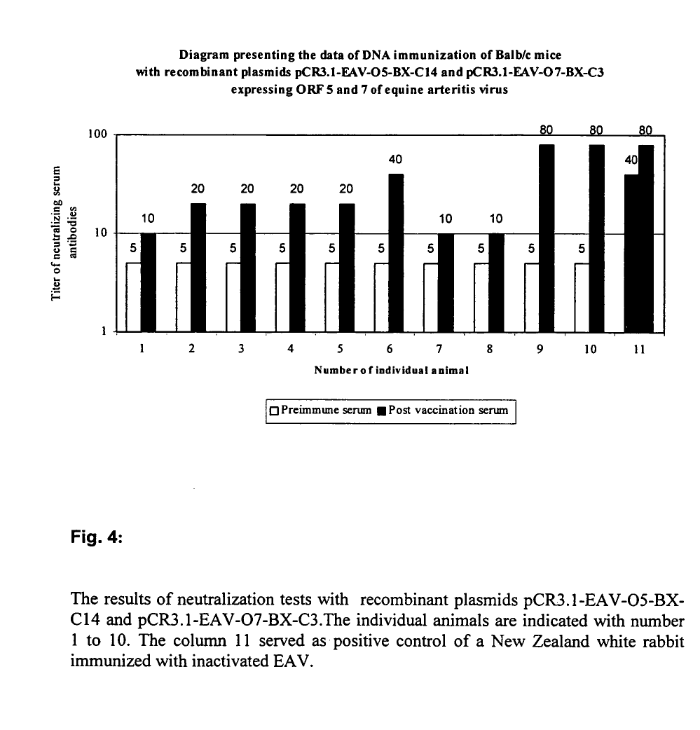

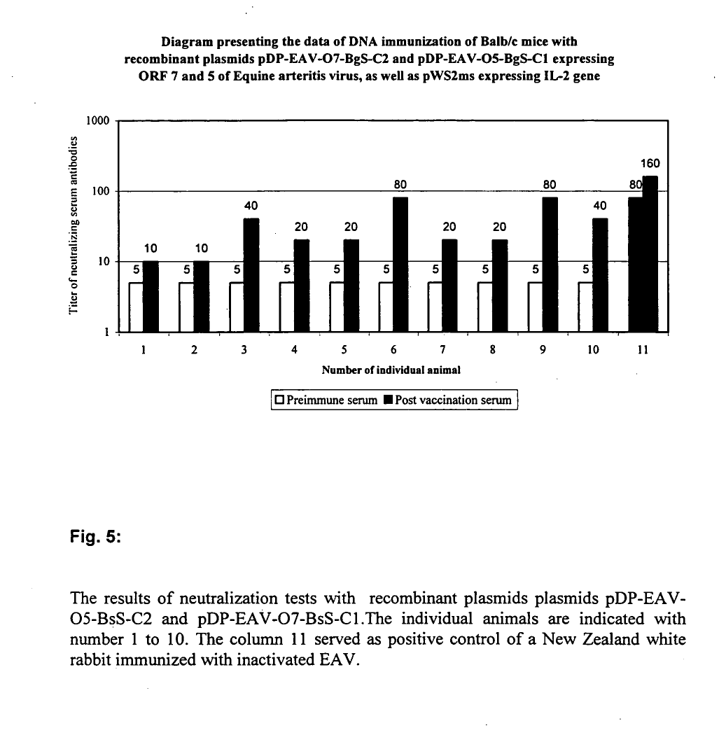

In the next step of this investigation the animals were administered with the DNAs of the recombinant plasmids pCR3.1-EAV-O7-BX-C3 and pCR3.1-EAV-O5-C14 simultaneously under the same conditions described above. The results of this study that is in agreement with the corresponding results obtained from the analysis of the gene products of the EAV ORFs 7 and 5 (Table 5 and FIG. 3) are summarized in Table 5 and shown in FIG. 4. These data indicate that the native DNA of recombinant plasmids pCR3.1-EAV-O7-BX-C3 (harboring ORF 7) and pCR3.1-EAV-O5-C14 (harboring ORF 5) is able to express the corresponding gene products (viral nucleocapsid protein and large envelope glycoprotein (G_{L})) in vivo.

The results of these studies unambiguously underline that the gene products of the EAV ORFs 7 and 5 are suitable candidates for development of a DNA vaccine protecting an EAV infection.

DNA Vaccination of Mice with Vector Construct Expressing Viral ORFs 5, 7 and L-2

In order to determine whether or not the L-2 is able to enhance the immunogenic potential of the gene product of the EAV ORFs 5 and 7 by DNA vaccination the following experiments were performed. An expression vector (pWS2 ms) containing the murine IL-2 expression cassette (kindly provided by Dr. W. Schmidt, Intercell, Vienna) was co-inoculated with two novel constructed recombinant plasmids pDP-EAV-O7-BgS-C2 and pDP-EAV-O5-C1 harboring and expressing the EAV ORFs 7 and 5 that encode the viral nucleocapsid protein and large envelope glycoprotein (G_{L}), respectively. The construction of these expression vectors based on pDisplay system that is similar to pCR3.1. This system additionally allows the cell surface expression of inserted epitopes by fusion to an N-terminal signal peptide and a C-terminal transmembrane domain. Balb/c mice were inoculated subcutaneously and intramuscularly for four times with 100 μg of each of the vector constructs in 14-day intervals. Under the conditions used an enhanced EAV-specific immune response in mice was detectable as shown in Table 5.

DNA Vaccination of Mice with Vector Construct Expressing Viral ORFs 5 and 6

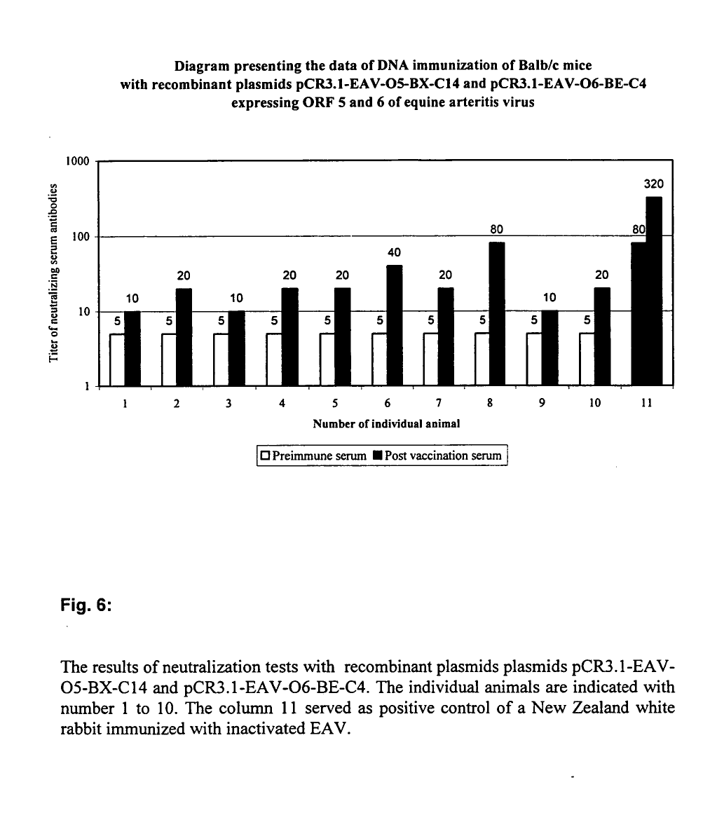

The EAV consists of an unglycosylated membrane protein (M, 17 kDa, gene product of ORF 6). It was of particular interest to proof whether or not this viral gene product can eventually influence and/or enhance the immunogenic potential of the gene products of EAV ORF 5 in vivo. A recombinant plasmid pCR3.1-EAV-O6-BE-C3 was constructed in which the translation unit of the EAV ORF 6 was inserted into the corresponding site of the expression vectors pCR3.1. Balb/c mice were inoculated with recombinant plasmids pCR3.1-EAV-O6-BE-C3 and pCR3.1-EAV-O5-C14 under the conditions described above. The sera obtained from the Balb/c mice (pre and post DNA vaccination) were analyzed using neutralization test and the results are given in Table 5 and shown in FIG. 6. A significant enhanced potential of the gene product of the EAV ORF 6 on the functional activities of the viral large envelope glycoprotein (gene product of EAV ORF 5) in vivo was not observed.

DNA Vaccination of Mice with Vector Construct Expressing Viral ORFs 3 and 4

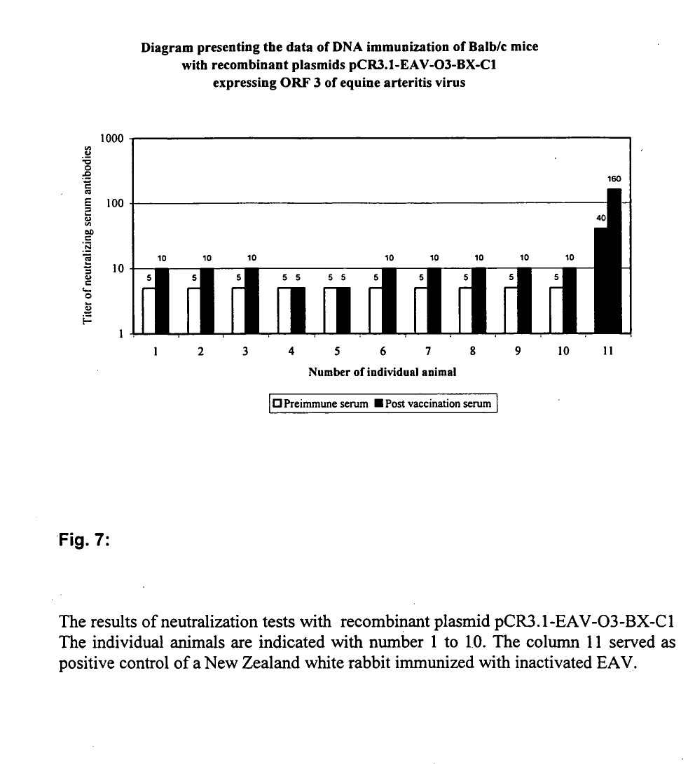

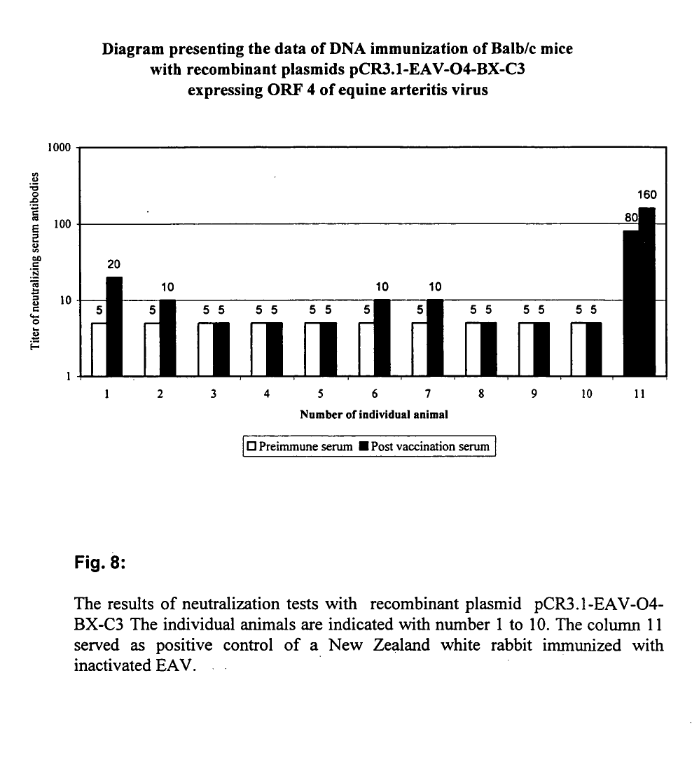

Although the functional activity of the gene products of two EAV ORFs 3 and 4 is not known, the eventual activity of these viral proteins in vivo was investigated. Two recombinant plasmids pCR3.1-EAV-O3-BX-C1 and pCR3.1-EAV-O4-BX-C3 was constructed (Table 3) in which the cDNA of the EAV ORFs 3 and 4 were inserted into the corresponding site of the expression vector pCR3.1. The sera obtained from the Balb/c mice (pre- and post DNA-vaccination) that were inoculated with these recombinant plasmids were analyzed using neutralization test. The results of these studies are summarized in Table 5 and shown in FIGS. 7 and 8 for the gene products of EAV ORFs 3 and 4, respectively. No neutralizing antibodies were detected in the immunized Balb/c mice under the conditions used.

DNA Vaccination of Mice with Vector Construct Expressing EAV N-Terminal Hydrophilic Ectodomain of Large Envelope Glycoprotein (G_{L})

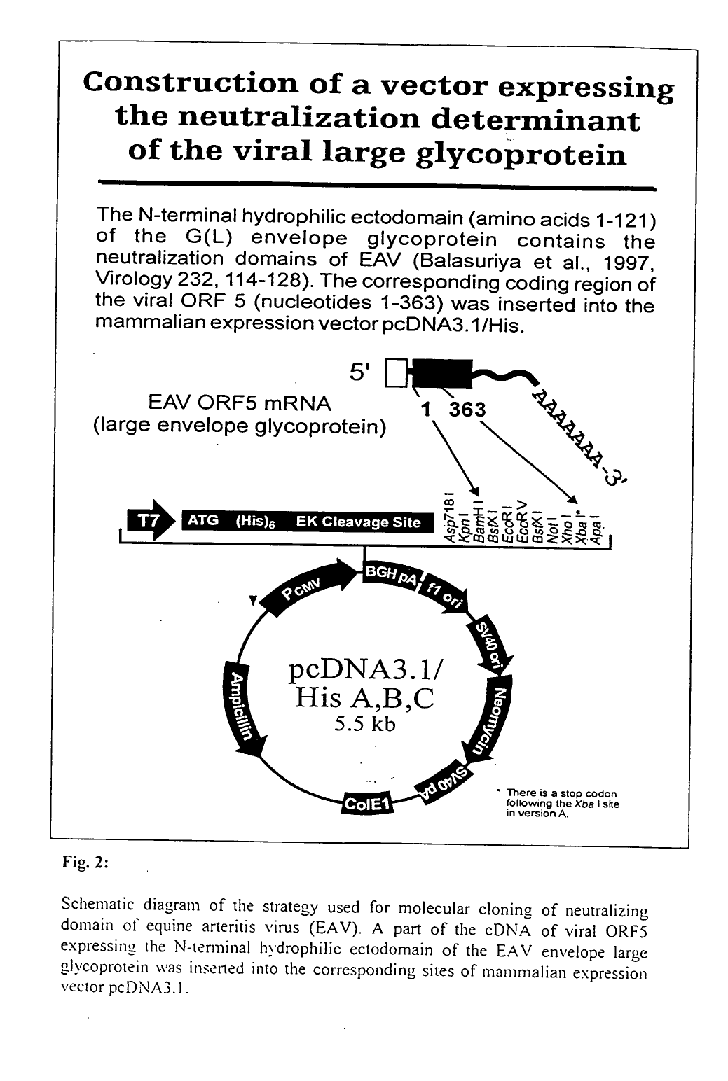

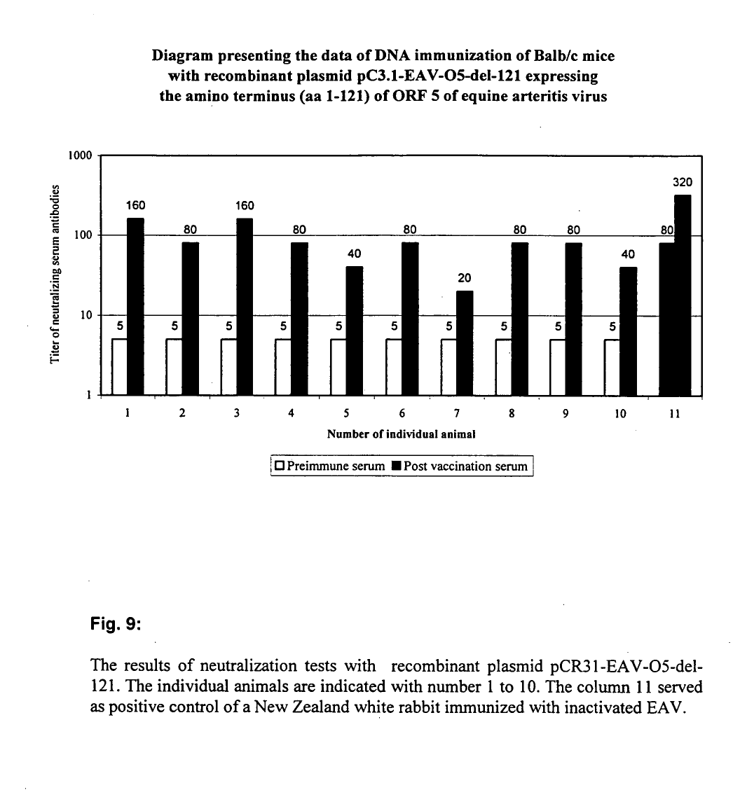

Balasuriya and coworkers (1995) found that the neutralization determinants of EAV are located on the gene product of ORF 5 (large envelope glycoprotein) within the N-terminal ectodomain (amino acid positions 1-121; Balasuriya et al., 1997). Accordingly, a novel expression vector based on pcDNA3.1/His system was constructed. This vector is similar to pCR3.1, but allows the specific detection and purification of the expressed fusion proteins using N-terminal amino acid sequence tags. The strategy of this experiment is shown in FIG. 2. A recombinant plasmid was constructed and termed pC31-HIS-EAV-O5-del-121. The insert of this expression vector possesses the coding region of EAV ORF 5 corresponding to amino acids 1-121 of the viral large envelope glycoprotein which was inserted into the corresponding sites of the parental vector pcDNA3.1-HIS-C (Table 5 and FIG. 2). Balb/c mice were inoculated with recombinant plasmid pC31-HIS-EAV-O5-del-121 under the conditions described above. The sera obtained from the Balb/c mice (pre and post DNA vaccination) were analyzed using neutralization test. As shown in Table 5 and FIG. 9 it was found that the majority of the DNA vaccinated animals (90%) developed a significant neutralizing antibody against EAV.

DNA Vaccination of Mice with Vector Constructs Expressing Viral Membrane and Glycoproteins in Combination with IL-2 Gene Expression

As described above, it had been shown that the viral N-glycosylated major membrane protein (G_{L}), the N-glycosylated minor membrane protein (G_{S}, 25 kDa), and unglycosylated membrane protein (M, 17 kDa) are able to develop significant immune response in vivo as detected by DNA vaccination of Balb/c mice. Furthermore, the enhancer function of IL-2 gene expression during DNA vaccination is known. However, an intermolecular interaction of these proteins during gene expression in vivo e.g. by DNA vaccination is not known, so far. Therefore it was of particular importance to proof whether or not these proteins are capable to induce an adequate immune response by simultaneous expression in vivo.

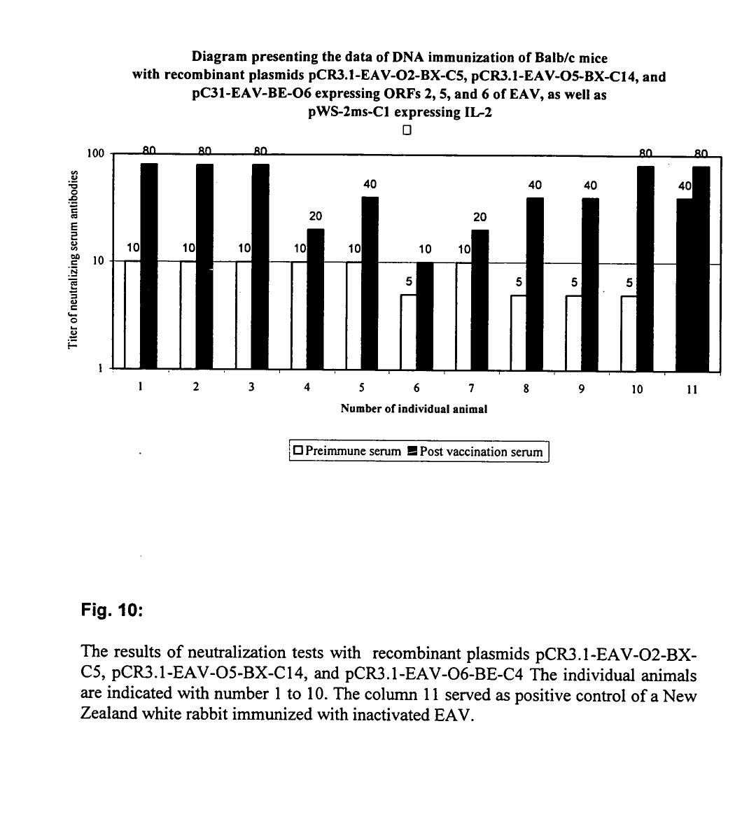

Four recombinant plasmids pCR3.1-EAV-O6-BE-C4, pCR3.1-EAV-O₂-BX-C5, pC3.1-EAV-O5-BX-C14, and pWS2 ms were used. These vector constructs are able to express viral membrane protein, glycoproteins G_{S}, G_{L}, and mice interleukin 2, respectively. Balb/c mice were inoculated with a DNA mixture containing the four recombinant plasmids under the standard conditions described above. The sera obtained from the Balb/c mice (pre- and post DNA vaccination) were analyzed using neutralization test and the corresponding data are summarized in Table 5 and shown in FIG. 10. Under the conditions used in this experiment 90% of the DNA vaccinated mice developed significant immune response against EAV.

DNA Vaccination of Mice with Vector Constructs Expressing Viral Small Glycoprotein and the Gene Product of the ORF 4

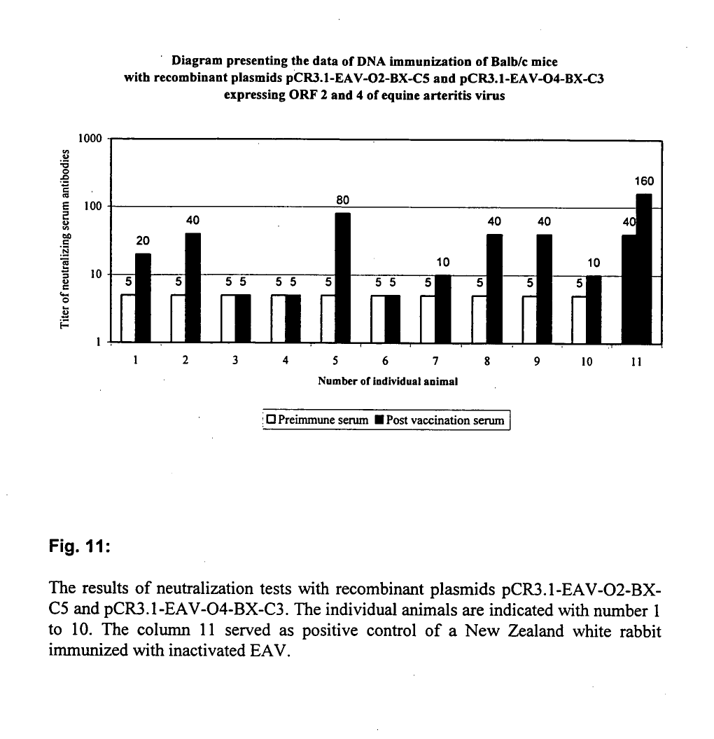

The capability of the EAV small glycoprotein (Gene products of ORF 2) and the gene product of ORF 4 to develop immune response in vivo was investigated by DNA vaccination of Balb/c mice. Two recombinant plasmids pCR3.1-EAV-O₂-BX-C5 and pCR3.1-EAV-O4-BX-C3 were used. These vector constructs are able to express viral small glycoproteins G_{S}, and the gene product of EAV ORF 4, respectively. Balb/c mice were inoculated with a DNA mixture containing the two recombinant plasmids under the standard conditions described above. The sera obtained from the Balb/c mice (pre and post DNA vaccination) were analyzed using neutralization test. As shown in FIG. 11 and Table 5, it was found that about 50% of the DNA vaccinated animals developed NT-titer against EAV.

Logistics for DNA Vaccination of Horses

In addition to the objective of our studies, it was necessary to develop and establish a novel screening system for detection of humoral and cellular immune response in vaccinated horses. An enzyme linked immunosorbent assay (ELISA) and a lymphocyte activation assay were established as described in the session of material and methods. Furthermore, it was necessary to prepare a convenient kit for vaccination of horses with the constructed expression vectors harboring and expressing the viral gene products of ORFs 2 to 7.

Development of a Novel Enzyme Linked Immunosorbent Assay (ELISA)

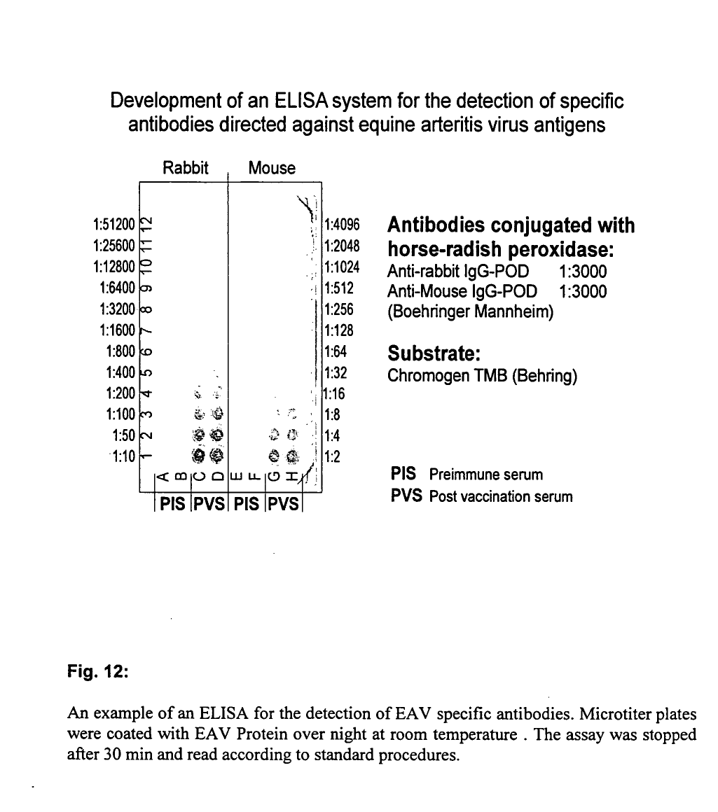

Polysorp F8 Microtiter Plates were coated with Protein of EAV infected RK-13 cells at a concentration of 2 μg×ml⁻¹. In this system antibodies against EAV (rabbit, mouse, and horse serum, etc.) can be detected. Horse-radish peroxidase labeled rabbit-anti IgG or horse-radish peroxidase labeled mouse-anti IgG served as second antibody. The color was developed by adding Buffer/Substrate TMB and Chromogen TMB and the adsorbance was determined according to standard procedures at 450 nm on an automatic ELISA reader (for detail see session material and methods). An example of the results of this analysis is shown in FIG. 12. The viral antigens prepared for ELISA can be used for performance of about 20,000 ELISA assays.

Development of a Lymphocyte Activation Assay

A Lymphocyte activation assay for the eventual use of the cellular response of the immunized mice was established. BALB/c mouse, female, anti-mouse CD3ε (500A2, PharMingen, Cat. No. 01511D), anti-mouse CD69 (H1.2F3, PharMingen, Cat. No. 01505B), Geys buffer (140 mM NH₄Cl, 2.7 mM KCl, 6.5 mM Na₂HPO₄, 1.5 mM KH₂PO₄, 0.7 mM CaCl₂, 0.5 mM MgCl₂), FACS buffer (PBS, 2% FCS, 0.01% NaN₃), Polystyrene round bottom tubes (Falcon, Becton Dickinson, Cat. No. 2052), and six well plate (TPP, Cat. No. 9206) were used. The measurement of the samples was performed using FACScan (Becton Dickinson), five measurable parameters: three high performance secondary photo multiplier with band pass filter: 530 nm for fluorescein isothiocyanate (FITC), 585 nm for phycoerythrin (PE) and 650 nm (red fluorescence), forward and side scatter.

One mouse was killed, spleenectomy was performed and spleen was transferred to a sterile tube with 1×BME medium. Intact spleen cells were separated and were washed with 1×BME medium. The solution was centrifuged for 3 min with 1500 rpm. The pellet was dissolved in 1.5 ml Geys buffer. After that the solution was centrifuged for 3 min with 1500 rpm. The pellet was dissolved in 9 ml BME medium with 10% FCS. One 6 well plate was filled with 1 ml 1×BME medium with 10% FCS, in 3 wells of the plate additional 1 μg/ml anti-mouse CD3ε was added. 1.5 ml cell solution was added to each well. The 6 well plate was incubated at 37° C., 5% CO₂ for three days. Solutions with activated and not activated cell were each divided in four Polystyrene round bottom tubes. The tubes were filled with FACS buffer and centrifuged for 3 min with 1500 rpm. The supernatant was discarded and the pellet was resuspended in two of four tubes with 100 μl anti-mouse CD3ε (1:100 diluted in FACS buffer) and incubated for 30 minutes at 4° C. For double fluorescence the cells were centrifuged for 3 min with 1500 rpm, the pellet was resuspended with 100 μl anti-mouse CD69 (1:100 diluted in FACS buffer) and incubated for 30 minutes at 4° C. again. The cells were centrifuged for 3 min with 1500 rpm, the pellet was resuspended in 2 ml FACS buffer, centrifuged again for 3 min with 1500 rpm, and resuspended in 500 μl FACS buffer each, vortexed and measured in the FACScan.

TABLE 1
An example of experimental protocol used for production
of polyclonal antibodies against equine arteritis
virus in New Zealand white rabbit

| Date | Treatment |
|---|---|
| 09.06.1998 | Preimmun-serum |
| | 10 × 620 μl |
| 10.06.1998 | 1^{st} inoculation* |
| | 0.5 ml/s.c. |
| 24.06.1998 | 2^{nd} inoculation* |
| | 0.5 ml/s.c. |
| 08.07.1998 | 3^{rd} inoculation* |
| | 0.5 ml/s.c. |
| 22.07.1998 | 4^{th} inoculation* |
| | 0.5 ml/s.c. |
| | First bleeding (10 ml) |
| | Titer by immunoblot analysis = 1:200 |
| 30.07.1998 | 5^{th} inoculation* |
| | 0.5 ml/s.c. |
| 11.08.1998 | Final bleeding (30 × 1.5 ml) |
| | Titer by immunoblot analysis => 1:2000 |

Virus: Equine arteritis virus: Virion in 0.5 ml PBS, purified by sucrose gradient centrifugation

Animal: New Zealand white rabbit (ca 2 kg/female)

TABLE 2

List of oligonucleotide primers that were
used for the amplification and molecular
cloning of equine arteritis virus cDNA.
Artificial sequences containing
restriction endonuclease recognition sites
(underlined) are shown in boldface
letters.

| | |
|---|---|
| Primer Name | Nucleotide Sequence |

| | |
|---|---|
| P-XbaI-EAV-O5- | 5′-GGGTCTAGAGTCACCACAAAATGAATCTATG |
| Rl | GC-3′ |
| P-BamHI-EAV-O5- | 5′-GGGGGATCCTGGTACGTTGGGCTCAACGAT |
| Fl | G-3′ |
| P-XbaI-EAV-O7- | 5′-GGGTCTAGACCACACAGGAGAATATCCACGT |
| Rl | C-3′ |
| P-BamHI-EAV-O7- | 5′-GGGGGATCCCGCAGTTGGTAACAAGCTTGTC |
| Fl | G-3′ |
| P-XbaI-EAV-O5- | 5′-GGGTCTAGAGTCACCACAAAATGAATCTATG |
| Rl | GC-3′ |
| P-BamHI-EAV-O5- | 5′-GGGGGATCCTGGTACGTTGGGCTCAACGAT |
| Fl | G-3′ |
| P-BamHI-EAV-O2- | 5′-GGGGGATCCATTTCTGTGATTGATGCAGCG |
| Fl | C-3′ |
| P-XbaI-EAV-O2- | 5′-GGGTCTAGAGGCATATTCATAACCCGTGTGG |
| Rl | ACTAC-3′ |
| P-BamHI-EAV-O3- | 5′-GGGGGATCCTTTGACCGGACCGGCCACATGG |
| Fl | GTC-3′ |
| P-XbaI-EAV-O3- | 5′-GGGTCTAGAGTAAGTAAAATTACGAGCCTCT |
| Rl | GCAGC-3′ |
| P-BamHI-EAV-O4- | 5′-GGGGGATCCCCTTTGTAGATGAAGATCTACG |
| Fl | GC-3′ |
| P-XbaI-EAV-O4- | 5′-GGGTCTAGAGCAATACAATCATAGATAACAT |
| Rl | CGTTGAGCCC-3′ |
| P-BamHI-EAV-O6- | 5′-GGGGGATCCCAGCTGAGGTATGGGAGCCATA |
| Fl | G-3′ |
| P-EcoRI-EAV-O6- | 5′-GGGGAATTCCATGCGCAGTAGGTCATTGTAG |
| Rl | C-3′ |
| P-BamHI-EAV-O5- | 5′-GGGGGGATCCTGGTACGTTGGGCTCAACGAT |
| Fln | G-3′ |
| P-XbaI-EAV-O5- | 5′-GGGGGTCTAGAGTCACCACAAAATGAATGTA |
| Rln | TGGC-3′ |
| P-BamHI-EAV-O7- | 5′-GGGGGGATCCCGCAGTTGGTAACAAGCTTGT |
| Fln | CG-3′ |
| P-XbaI-EAV-O7- | 5′-GGGGGTCTAGACCACACAGGAGAATATCCAC |
| Rln | GTC-3′ |
| P-SalI-EAV-O5- | 5′-GGGGTCGACGTCACCACAAAATGAATCTATG |
| Rl | GC-3′ |
| P-EcoRI-EAV-O7- | 5′-GGGGAATTCCACACAGGAGAATATCCACGT |
| Rl | C-3′ |
| P-EAV-O5-Rl | 5′-CAACTATGCCGAATTCACGGCC-3′ |
| P-EAV-O5-Fl | 5′-GGCCGTGAATTCGGCATAGTTG-3′ |
| P-BamHI-EAV-O2- | 5′-GGGGGGATCCATTTCTGTGATTGATGCAGCG |
| Fln | C-3′ |
| P-XbaI-EAV-O2- | 5′-GGGGGTCTAGAGGCATATTCATAACCCGTGT |
| Rln | GCACTAC-3′ |
| P-BamHI-EAV-O3- | 5′-GGGGGGATCCTTTGACCGGACCGGCCACATG |
| Fln | GGTC-3′ |
| P-XbaI-EAV-O3- | 5′-GGGGGTCTAGAGTAAGTAAAATTACGAGCCT |
| Rln | CTGCAGC-3′ |
| P-BamHI-EAV-O4- | 5′-GGGGGGATCCCCTTTGTAGATGAAGATCTAC |
| Fln | GGC-3′ |
| P-XbaI-EAVO4- | 5′-GGGGGTCTAGAGCAATACAATCATAGATAAC |
| Rln | ATCGTTGAGCCC-3′ |
| P-BamHI-EAV-O6- | 5′-GGGGGGATCCCAGCTGAGGTATGGGAGCCAT |
| Fln | AG-3′ |
| P-EcoRI-EAV-O6- | 5′-GGGGGGAATTCCATGCGCAGTAGGTCATTGT |
| Rln | AGC-3′ |
| P-SalI-EAV-O5- | 5′-GGGGGGGTCGACTGGCTCCCATACCTCAGCT |
| Rln | GC-3′ |
| P-BglII-EAV-O7- | 5′-GGGGGGAGATCTATGGCGTCAAGACGATCAC |
| Fln | GTCCG-3′ |
| P-SalI-EAV-O7- | 5′-GGGGGGGTCGACCGGCCCTGCTGGAGGCGCA |
| Rln | AC-3′ |
| P-BglII-EAV-O2- | 5′-GGGGGGAGATCTATGCAGCGCTTTTCTTTCT |
| Fln | CATGC-3′ |
| P-SalI-EAV-O2- | 5′-GGGGGGGTCGACCAAAATCTTGCGCCGCGGC |
| Rln | AAAG-3′ |
| P-BglII-EAV-O6- | 5′-GGGGGGAGATCTATGGGAGCCATAGATTCAT |
| Fln | TTTGTGG-3′ |
| P-SalI-EAV-O6- | 5′-GGGGGGGTCGACTTGTAGCTTGTAGGCTGTC |
| Rln- | GCCG-3′ |
| P-EcoRI-EAV-O1- | 5′-GGGGGGGAATTCATGGCAACCTTCTCCGCTA |
| Fln | CTGG-3′ |
| P-XbaI-EAV-O1- | 5′-GGGGGGTCTAGATCACACGGGCCCAATGACT |
| Rln | GAACC-3′ |
| P-AflII-TOEAV- | 5′-CCCCTTAAGTGCCATATACGGCTCACCACCA |
| Fl | TATACAC-3′ |
| P-MroI-TOEAV-Rl | 5′-CCCTTCCGGAGGTTCCTGGGTGGCTAATAAC |
| | TACTTC-3′ |
| PR-BAM-5102 | 5′-GGGGGGATCCCAGTACTTTGGC-3′ |
| PF-BAM-5097 | 5′-GGGGGGATCCCAGCGACACCCGAGGCAC |
| | G-3′ |
| PR-SPH-8570 | 5′-GGGGGGCATGCTGTAGGTGGGCCATGGATCA |
| | AGTCC-3′ |
| PF-SPH-8565 | 5′-GGGGGGCATGCCACCTGGGCCAAGAAATTGA |
| | CC-3′ |
| PR-EAV-6959- | 5′-GCTGGCCGCGTAATGCTGGTTGGG-3′ |
| 6946 | |
| PF-EAV-5948- | 5′-CCGTGCTGGATGTGAGGCCGTTACCG-3′ |
| 5973 | |
| P-EAV-GL-ECTO-F | 5′-GGGGGGATCCATGTTATCTATGATTGTATTG |
| | CTATTC-3′ |
| P-EAV-GL-ECTO-R | 5′-GGGGGGTCTAGACAACACAACTATGCCGAAT |
| | TCAC-3′ |
| P-pcDNA3.1/ | 5′-ATGGTATGGCTAGCATGACTGGT-3′ |
| HisC-F | |
| P-pcDNA3.1/ | 5′-CAAACAACAGATGGCTGGCAACT-3′ |
| HisC-R | |

TABLE 3
Properties of the constructed mammalian expression vectors harboring
EAV-specific cDNA of the individual translations units.

| | EAV-Specific Insert | Expression Vector | Oligonucleotide |
|---|---|---|---|
| Construct Name | (Nucleotide Position) | (Restriction Sites) | Primers |
| pCR3.1-EAV-O2-BX | ORF 2: small | pCR3.1 | P-BamHI-EAV-02-F1n |
| | glycoprotein | (BamHI/XbaI) | P-XbaI-EAV-02-R1n |
| | (9807-10490) | G/GATCC/T/CTAGA | |
| pCR3.1-EAV-O3-BX | ORF3 | pCR3.1 | P-BamHI-EAV-03-F1n |
| | (10289-10780) | (BamHI/XbaI) | P-XbaI-EAV-03-R1n |
| | | G/GATCC/T/CTAGA | |
| pCR3.1-EAV-O4-BX | ORF4 | pCR3.1 | P-BamHI-EAV-04-F1n |
| | (10683-11141) | (BamHI/XbaI) | P-XbaI-EAV-04-R1n |
| | | G/GATCC/T/CTAGA | |
| pCR3.1-EAV-O5-BX | ORF 5: large | pCR3.1 | P-BamHI-EAV-05-F1n |
| | glycoprotein | (BamHI/XbaI) | P-XbaI-EAV-05-R1n |
| | (11129-11896) | G/GATCC/T/CTAGA | |
| pDP-EAV-O5-BgS | ORF 5: large | PDisplay | P-BglII-EAV-05-F1n |
| | glycoprotein | (BglII/SalI) | P-SalI-EAV-05-R1n |
| | (11129-11896) | A/GATCT/G/TCGAC | |
| pCR3.1-EAV-O6-BE | ORF 6: membrane protein | pCR3.1 | P-BamHI-EAV-06-F1n |
| | (11884-12372) | (BamHI/EcoRI) | P-EcoRI-EAV-06-R1n |
| | | G/GATCC/G/AATTC | |
| pCR3.1-EAV-O7-BX | ORF 7: | pCR3.1 | P-BamHI-EAV-07-F1n |
| | nucleocapsid protein | (BamHI/XbaI) | P-XbaI-EAV-07-R1n |
| | (12296-12628) | G/GATCC/T/CTAGA | |
| pDP-EAV-O7-BgS | ORF 7: | PDisplay | P-BglII-EAV-07-F1n |
| | nucleocapsid protein | (BglII/SalI) | P-SalI-EAV-07-R1n |
| | (12296-12628) | A/GATCT/G/TCGAC | |
| pCR3.1-HIS-EAV- | partial ORF 5: | pcDNA3.1/HIS C | P-EAV-GL-ECTO-F |
| O5-del-1-121-BX | C-terminal ectodomain of | (BamHI/XbaI) | P-EAV-GL-ECTO-R |
| | large glycoprotein | G/GATCC/T/CTAGA | |
| | (11129-11492) | | |

TABLE 4
Properties of the equine arteritis virus specific cDNA obtained by long range RT-PCR

| | | Size of | Site (s) of | |
|---|---|---|---|---|
| PCR product | Nucleotide | Viral cDNA | Restriction | Oligonucleotide |
| (viral ORFs) | Position | Fragment bp | Enzyme (s) | Primers |
| PCR-EAV-1-5102 | 1-5102 | 5102 | AflII/BamHI | P-AflII-TOEAV-F1 & |
| (truncated ORF 1 a*) | | | C/TTAAG/G/GATCC | PR-BAM-5102 |
| PCR-EAV-5097-8570 | 5097-8570 | 3473 | BamHI/SphI | PF-BAM-5097 & |
| (Part of ORF1 a & b*) | | | G/GATCC/GCATG/C | PR-SPH-8570 |
| PCR-EAV-8565-12687 | 8565-12687 | 4122 | SphI/MroI | PF-SPH-8565 & |
| (Part of ORF1 b & 2-7**) | | | GCATG/C/T/CCGGA | P-Mro I-TOEAV-R1 |
| PCR-EAV-1-8570 | 1-8570 | 8570 | AflII/SphI | P-AflII-TOEAV-F1 & |
| (ORF 1 a & truncated 1b*) | | | C/TTAAG/GCATG/C | PR-SPH-8570 |
| PCR-EAV-5097-12687 | 5097-12687 | 7590 | BamHI/MroI | PF-BAM-5097 & |
| (Part of ORF1a*-7**) | | | G/GATCC/T/CCGGA | P-Mro I-TOEAV-R1 |
| PCR-EAV-1-6959 | 1-6959 | 6959 | AflII | P-AflII-TOEAV-F1 & |
| (ORF1 a & part of 1b*) | | | C/TTAAG | PR-EAV-6959-6946 |
| PCR-EAV-5948-12687 | 5948-12687 | 6739 | MroI | PF-EAV-5948-5973 & |
| (Part of ORF1b*-7**) | | | T/CCGGA | P-Mro I-TOEAV-R1 |

*ORF1: ORF 1a: 208-5391, ORF 1b: 5388-9734 nucleotides
**ORF2-7: 9807-12628 nucleotides

TABLE 5
Summary of the results obtained by DNA immunization of mice
using different gene products of equine arteritis virus

| | | Average | | |
|---|---|---|---|---|
| | | of NT-AB^{a} titer | | |
| Experiment | Expression Vector | minimum/maximum | | % of |
| (No. of animals) | (ORF/Gene product) | PIS | PVS | Immunresponse^{b} |
| MV-00-01(10) | pCR3.1 | <1:10 | <1:10 | 0 |
| 07/27/1998 to | | | | |
| 10/19/1998 | | | | |
| MV-O7-01(10) | pCR3.1-EAV-O7-BX-C3 | <1:10/1:10 | 1:20/1:640 | 80 |
| 07/27/1998 to | (ORF 7/nucleocapsid | | | |
| 10/19/1998 | protein = NP) | | | |
| MV-O5-01(10) | pCR3.1-EAV-O5-BX-C14 | <1:10 | 1:10/1:160 | 70 |
| 08/29/1998 to | (ORF 5/large envelope | | | |
| 11/11/1998 | glycoprotein = G_{L}) | | | |
| MV-O5-02(10) | pCR3.1-EAV-O5-BX-C14 | <1:10 | 1:10/1:160 | 50 |
| 12/07/1998 to | (ORF 5/G_{L}) | | | |
| 03/20/1999 | | | | |
| MV-57-01(10) | pCR3.1-EAV-O5-BX-C14 | <1:10 | 1:10/1:80 | 30/70^{c} |
| 09/11/1998 to | (ORF 7/NP) | | | |
| 11/23/1999 | pCR3.1-EAV-O7-BX-C3 | | | |
| | (ORF 5/G_{L}) | | | |
| MV-57IL2-1 (10) | pDP-EAV-O5-BgS-C1 | <1:10/1:10 | 1:10/1:80 | 40/80^{c} |
| 10/26/1998 to | (ORF 7/NP) | | | |
| 01/25/1999 | pDP-EAV-O7-BgS-C2 | | | |
| | (ORF 5/G_{L}) | | | |
| | pWS2ms (IL2) | | | |
| MV-56-01(10) | pCR3.1-EAV-O5-BX-C14 | <1:10 | 1:10/1:80 | 20/70^{c} |
| 12/07/1998 to | (ORF 7/NP) | | | |
| 02/08/1999 | pCR3.1-EAV-O6-BE-C4 | | | |
| | (ORF 6/membrane protein) | | | |
| MV-O3-01(10) | pCR3.1-EAV-O3-BX-C1 | <1:10 | <1:10/1:10 | 0 |
| 12/21/1998 to | (ORF 3/?) | | | |
| 03/22/1999 | | | | |
| MV-O4-01(10) | pCR3.1-EAV-O4-BX-C3 | <1:10 | <1:10/1:20 | 0 |
| 12/22/1998 to | (ORF4/?) | | | |
| 03/22/1999 | | | | |
| MV-O5d-01(10) | pC3.1-HIS-EAV-O5-del-121 | <1:10 | 1:20/1:160 | 90/100^{c} |
| 12/29/1998 to | (amino terminus of | | | |
| 03/22/1999 | large envelope | | | |
| | glycoprotein; the first | | | |
| | 121 amino acids) | | | |
| MV-256IL2-1(10) | pCR3.1-EAV-O2-BX-C5 | <1:10/1:10 | 1:10/1:80 | 70/90^{c} |
| 02/12/1999 to | (ORF 2/small glycoprotein) | | | |
| 04/07/1999 | pCR3.1-EAV-O5-BX-C14 | | | |
| | (ORF 5/G_{L}) | | | |
| | pCR3.1-EAV-O6-BE-C4 | | | |
| | (ORF 6/membrane protein) | | | |
| | pWS2ms (IL2 gene) | | | |
| MV-99/5(10) | pCR3.1-EAV-O2-BX-C5 | <1:10/1:10 | <1:10/1:80 | 40/50^{c} |
| 05/14/1999 to | (ORF 2/small | | | |
| 07/09/1999 | glycoprotein) | | | |
| | pCR3.1-EAV-O4-BX-C3 | | | |
| | (ORF4/?) | | | |

^{a}Neutralizing antibodies
^{b}Neutralizing titer 1:20 excluded
^{c}Neutralizing titer 1:20 included
PIS: Preimmune serum;
PVS: Post vaccination serum
?Protein of unknown function

### EXAMPLE 2

### DNA Vaccination of Horses

Preparation of a DNA Vaccine Kit for Application in Horses

A convenient kit for vaccination of horses harboring the individual constructed recombinant plasmid (10 mg DNA) expressing the viral gene products of ORFs 2 to 7 was prepared. The properties of the DNA vaccination kit are summarized in Table 6. In order to proof the ability of the expression of EAV cDNAs of ORFs 2 and 5 to 7 in the autologous animal system horse it was necessary to screen the horse sera prior to immunization trial. The selection of the suitable animals was based on the results obtained from the analysis of the horse sera. These studies allowed detecting the existence of a previous naturally occurred EAV infection in horses. Five sera were obtained from Prof. Dr. H. Müller (Institut für Virologie, Veterinärmedizinische Fakultät, Universität Leipzig). The horse sera were analyzed by neutralization assay and ELISA as described above. The results of these experiments for sera obtained from Leipzig are summarized in table 7. In contrast, it was found that three horses from the animal farms of Leipzig did not developed specific antibodies against an EAV infection. Consequently, it seem to be rational that the animals from the later farm can be considered for evaluation of EAV DNA vaccine in its natural host.

Preparation of Autologous Skin Fibroblasts

Two skin biopsies were performed from each of the five horses involved in the study (Daggy, Frieda, Friedrich, Jessy, Nelke). Skin samples were cut into (5-8) slices and attached to cell culture flasks. After a 30 min incubation at 37° C. and 5% CO₂, skin samples were cultured in Dulbecco's modified eagles medium (DMEM) containing 10% fetal calf serum (FCS), antibiotics and antimycotics. Fibroblasts growing out of the skin samples were passaged twice and extended to 2×10⁷ cells. From each horse cells were frozen in liquid nitrogen (N2) in aliquots containing at least 2×10⁶ cells. Several aliquots were thawed and cultured in DMEM containing 10% FCS and antibiotics to verify the competence of these cells for further growth. Cells were passaged more than 20 times. For transfection experiments, however, cell of passage 15 and more proved to be not suitable.

Purification of Plasmid DNA

Plasmids were obtained from Boehringer Ingelheim (named pCR3.1-EAV-O₂-BX-C3), pCR3.1-EAV-O5-BX-C14, and pCR3.1-EAV-O7-BX-C3). After selection of ampicillin resistent e. coli K12 colonies, bacterial cultures containing the different plasmids were grown to large scale. From each plasmid 500 μl DNA at a concentration of 1.5 μg/μl were isolated and stored at −20° C. for the transfection experiments.

Transfection Experiments

Transfection experiments were initially performed in 12 and 24 well plates. Titration experiments in the 24 well plate format using cell numbers between 1.5×10⁴ and 3×10⁵ per well revealed that 1.25×10⁵ cells seeded in each well were almost confluent (85%) within 24 hours.

a) Lipofection as Transfection Reagents

Titration experiments performed in a 24 well plate format using 5-20 μl/well Lipofectin showed that amounts >15 μl were toxic to the cells. Titration experiments using different amounts (5-80 μg/well) of DNA with 12.5 μl Lipofectin revealed that concentrations of 80 μg DNA showed the highest transfection efficiencies. However, the efficiency in these transfection experiments did not exceed 10%.

b) LipofectAMINE as Transfection Reagent

In order to increase the transfection efficiency, transfection experiments were performed using additional transfection reagents (LipofectAMINE, LipofectAMINE plus, DMRIE). Compared to the results of the transfection experiments using Lipofectin. DMRIE did not result in higher number of transfected cells. LipotectAMINE showed to be more toxic to the cells, but the transfection efficiencies were higher. The recommdation of the supplier to use in addition to LipofectAMINE the ‘plus’ reagent did not increase the transfection rate. Therefore, LipofectAMINE seems to be the reagent of choice to transfect the cultured primary horse skin fibroblasts.

In the initial experiments using 7.5 μl LipofectAMINE and 5 μg of DNA, about 15% of the target cells were transfected. Currently transfection experiments are being performed to further increase the transfection efficiency. So far, the transfection efficiency is about 20%.

Isolation of Peripheral Blood Lymphocytes

Three weeks and one day before the initial immunisation, 50 ml of heparinised blood was collected from each horse by jugular venipuncture. The blood was centrifuged at 400×g for 5 min and the plasma was removed. Blood calls (10 ml) were resuspended with phosphate buffered saline (PBS) to a volume of 25 ml, layed on a Ficoll Hypaque gradient (15 ml) and centrifuged at 400×g for 30 min. The PBMC were collected from the interface, washed twice in PBS, counted and frozen in aliquots in N₂ in 10% DMSO and 90% FCS.

To test the viability of the PBMC, cells were thawed, cultured in Isocove's modified Eagles medium (IMEM) containing 10% FCS and antibiotics. PBMC were stimulated for two days with 2.5 μg/ml pookweed mitogen and cultured thereafter in the presence of 200U of human recombinant IL-2. PBMC showed a good proliferation, were grown to 2×10⁶ cells/ml and were frozen again as aliquots in N₂.

Detection of EAV-Specific Antibodies

Serum was collected 4 months, three weeks and one day before the initial immunisation. Serum of the first timepoint was investigated at the Institute of Medical Virology (Prof. Darai) in Heidelberg for the presence of EAV-specific antibodies. The results are summarised in table 10.

Immunization of the Horses

All five horses were immunised by intramuscular (i.m.) injection and intradermal application of the EAV plasmid DNA encoding (parts of) ORF 2, ORF 5, and ORF 7 using a gene gun. The i.m. inoculations were applied to the musculi semimembranosus/semitendinosus/gluteus. The gene gun application of the DNA was performed on both sides of the neck. The corresponding parts of the skin (40 cm²) were shaved before to enable a good DNA application using the gene gun. A detailed protocol of the DNA immunisation (including sedation of the horses, amount of DNA, adjuvants, buffers) is provided in example 4. The horses were investigated 24 and 48 h post immunisation for local and systemic reactions. None of the animals developed fever and the local reactions (thickness of the skin, development and involution of papules) are summarised in tables 12 and 13.

Collection of Blood and Serum Samples

Peripheral blood lymphocytes, serum and plasma of the horses were taken as outlined in Table 12. Up to date, blood and serum samples were taken three times after the 3^{rd} booster immunization to measure the kinetics of antibody titres and activities of cytotoxic T-lymphocytes.

Determination of the Maximum Cellular ⁵¹Cr-Uptake

Titration experiments were perfomed using (i) constant amounts of ⁵¹Cr with different cell-concentrations and incubation times and (ii) constant incubation times with different cell-concentrations and amounts of ⁵¹Cr.

Target cells (5×10³, 2×10⁴, and 5×10⁴) were labeled with 100 μCi for each 90 min., 150 min., and 240 min. The cellular ⁵¹Cr-uptake as well as ⁵¹Cr present in the culture supernatant were determined. In the supernatants of the labeled cultures, a linear increase of ⁵¹Cr was measured (FIG. 14a). The amount of cellular ⁵¹Cr also increased with longer incubation times and higher numbers of cells.

Similar results were obtained when these experiments were performed using constant incubation times with different cell concentrations and amounts of ⁵¹Cr (FIG. 14a). The substraction of the ⁵¹Cr present in the cells and the ⁵¹Cr released into the supernatant is shown in (FIG. 14b). For the measurement of cytotoxic T-lymphocyte activities in the blood of the immunized horses, it was decided to label 5×10⁴ target cells with 200 μCi for 240 min.

Measurement of Cytotoxic T-Lymphocyte Activities after the 2^{nd} and 3^{rd} Booster Immunization

The preparation of the target cells including transfection and ⁵¹Cr-labeling was performed as described in detail in Example 3. The isolation of the peripheral blood lymphocytes, the cell culture in vitro as well as the restimulation of the effector cells was also performed as outlined in Example 3.

As described above, we labeled 5×10⁴ target cells with 200 μCi for 240 min. To avoid spontaneous release of ⁵¹Cr by the cells the target cells were kept on ice for 45 min after the first washing step. The effector cells were added to the cultures using effector/target cell ratios of 3:1, 25:1, and 50:1. After incubation for 8 h at 37° C. and 5% CO₂ culture plates were centrifuged at 1000 RPM for 3 min and each supernatant was measured for the presence of ⁵¹Cr in a scintillation. Negative control culture supernatants consisted of labelled cells without the addition of effector cells (spontaneous ⁵¹Cr-release of the cells). Positive control culture supernatants consisted of cultures of labeled cells after cell lysis using 10% Triton X-100 (maximum ⁵¹Cr-uptake). The results of the ⁵¹Cr-release of the individual cultures including the effector cells prior to immunization, two weeks after the 2^{nd} and two weeks after the 3^{rd} booster immunisation (see Table 11) are included as table 18. FIGS. 15a)-15e) show the calculated specific lysis ≧0 according to Hammond S A, Issel C J, and Montelaro R C (1998): General method for the detection and in vitro expansion of equine cytolytic T lymphocytes. J Immunol Method 213: 73-85.

The data sheets containing the ⁵¹Cr-release of the individual cultures (table 18) show the relative small standard deviations between the four individually handled cultures of each sample. Compared with the earlier cytotoxic T-cell assays performed with cells after the first immunization and the 1^{st} booster immunization (see example 3), the differences between the negative control culture supernatants (spontaneous ⁵¹Cr-release of the cells) and the positive control culture supernatants (maximum ⁵¹Cr-uptake by the cells) are greater in the current assay. In general, e measured higher percentages of the calculated specific lysis (FIG. 15). In the cultures containing effector cells obtained prior to immunization, however, we also measured relatively high percentages of specific lysis. It would be expected to have higher amounts of ⁵¹Cr released in the supernatants when higher number of effector cells were added to the targets. The values measured in the supernatants of the cultures with effector/target cell ratios of 3:1, 25:1, and 50:1, however, did not show such a consistence in specific lysis. Possibly, the number of antigen expressing target cells (approximately 20%) are not high enough to enable the measurement of speicific lysis. To enlarge the number of antigen positive cells, we used in the current experiments 5×10⁴ target cells. Other possible reasons for the inconsistency of specific lysis are not efficient restimulations of the effector cells in vitro or other unknown methodical details.

Summary of the Measured Cytotoxic T-Lymphocyte Activities

Due to the inconsistency of the measured specific lysis it is difficult to draw conclusions concerning the antigen(s) most suited for the induction of cytotoxic T-lymphocytes. In an attempt to give an overview of the measured specific lysis we calculated for each ORF the average value (X) of the specific lysis of all different effector/target cell ratios (3:1, 25:1, 50:1) and subtracted the average value (Y) of the negative controls (results of the cells before immunization), which were calculated similarly.

$$X = \frac{{X}_{1} ( 3 \text{:} 1 ) + {X}_{2} ( 25 \text{:} 1 ) + {X}_{3} ( 50 \text{:} 1 )}{3} Y = \frac{{X}_{1} ( 3 \text{:} 1 ) + {Y}_{2} ( 25 \text{:} 1 ) + {Y}_{3} ( 50 \text{:} 1 )}{3}$$

The results show the absolute increase of the cell lysis in percent at the indicated times for each ORF, compared to the values obtained before immunization.

Two different scales were used to express the increase of specific lysis. In example 5, Table 19, measured lysis between 0 and 5% is given a +(plus). In example 5, Table 20, measured lysis between 0 and 5% is given a—(minus). Horse Daggy was serologically positive before immunization. This explains the measured specific lysis already two weeks after the first immunization. Horse ‘Frieda’ had a weak antibody response before the first immunization. Cytotoxic T-lymphocytes directed against the expression product of ORF 2 and 7 were also measured in the blood of this animal two weeks after the first immunization. The serologically negative animals ‘Nelke’ and ‘Friedrich’ did not develop cytotixic T-lymphocytes after the first immunization. Only in horse ‘Jessy’, also serologically negative before immunization, cytotoxic T-lymphocytes directed against the expression product of ORF 7 were measured. Unfortunately, the specific lysis was not consistent at the following time points. After the 3^{rd} booster immunization, however, specific lysis (>10%) eas measured in all animals. The activity of cytotoxic T-lymphocytes was directed in horse ‘Frieda’ and ‘Jessy’ against the expression product of ORF 5, in horse ‘Daggy’ and ‘Friedrich’ against the expression product of ORF 7 and in horse ‘Nelke’ against the expression product of ORF 7.

TABLE 6
Content of a kit produced for DNA-vaccination of
horses using expression vectors harboring and expressing
individual gene of equine arteritis virus

| | Concentration | Total of |
|---|---|---|
| | DNA | plasmid DNA |
| Expression vectors | μg/μl | mg |
| pCR3.1-EAV-O2-BX-C5 | 1 | 10 |
| Gene product: small glycoprotein | | |
| pCR3.1-EAV-O3-BX-C1 | 1 | 10 |
| Gene product: unknown | | |
| pCR3.1-EAV-O4-BX-C3 | 1 | 10 |
| Gene product: unknown | | |
| pCR3.1-EAV-O5-BX-C14 | 1 | 10 |
| Gene product: large envelope | | |
| glycoprotein | | |
| pCR3.1-EAV-O6-BE-C4 | 1 | 10 |
| Gene product: membrane protein | | |
| pCR3.1-EAV-O7-BX-C3 | 1 | 10 |
| Gene product: nucleocapsid protein | | |
| pDP-EAV-O5-BgS-C1 | 1 | 10 |
| Gene product: large envelope | | |
| glycoprotein | | |
| pDP-EAV-O7-BgS-C2 | 1 | 10 |
| Gene product: nucleocapsid protein | | |
| pC3.1-HIS-EAV-O5del-121-C12 | 1 | 10 |
| Gene product: The N-terminal (121 aa) | | |
| hydrophilic ectodomain of large | | |
| envelope glycoprotein | | |

TABLE 7
Test of EAV specific antibodies in preimmune serum
The results of neutralization test (NT) and ELISA test in which
the preimmune sera of 5 horses from Leipzig were screened for
existence of a humoral immune response developed during
a natural infection with wild type equine arteritis virus (EAV)

| | Titer of serum | Titer of serum | |
|---|---|---|---|
| Horse | NT^{a} | ELISA | Remarks |
| Daggi | 1:40 | 1:100 | positive |
| Frieda | 1:20 | 1:50 | boundary |
| Friedrich | 1:10 | <1:50 | |
| Jessy | 1:10 | <1:50 | |
| Nelke | 1:10 | <1:50 | |
| Positive | 1:160^{b} | | positive |
| Control | 1:80^{c} | 1:200^{c} | positive |
| Serum | | | |

^{a}Multiplicity of infection (MOI): 100 PFU of EAV/1000 RK-13 cells/well
^{b}Rabbit antiserum raised against EAV
^{c}Horse serum was obtained from Prof. Dr. Ludwig, Berlin

TABLE 8
Duration of Immunity: Determination of antibody titer by neutralization test (NT) in the serum of
5 horses after DNA vaccination with the cDNA EAV ORF 2, 5 and 7 and cDNA encoding equine IL 2.

| | Pre-vaccine | 1. Post-vaccine | 2. Post-vaccine | 3.Post-vaccine | 4. Post-vaccine | 5. Post-vaccine | 6. Post-vaccine |
|---|---|---|---|---|---|---|---|
| | Serum | Serum | Serum | Serum | Serum | Serum | Serum |
| | 22.5.2000 | 5.6.2000 | 28.7.2000 | 22.9.2000 | 19.1.2001 | 16.3.2001 | 11.5.2001 |
| Horse | NT-Titer: | NT-Titer: | NT-Titer: | NT-Titer: | NT-Titer: | NT-Titer: | NT-Titer: |
| Daggy | 1:64 | 1:256 | 1:256 | 1:264 | 1:128 | 1:96 | 1:16 |
| Frieda | <1:2 | 1:256 | 1:256 | 1:264 | 1:64 | 1:32 | 1:16 |
| Friedrich | <1:2 | 1:64 | 1:128 | 1:512 | 1:48 | 1:32 | 1:8 |
| Jessy | <1:2 | 1:256 | 1:128 | 1:128 | 1:16 | 1:16 | 1:8 |
| Nelke | 1:8 | 1:256 | 1:256 | 1:128 | 1:16 | 1:16 | 1:16 |

TABLE 9
Immunization schedule with the cDNA EAV ORF 2, 5 and 7 and with
cDNA encoding equine IL 2. Combined application of cDNA (in μg)
via Gene Gun (GG) and intramuscular injection (i.m.)

| | 1. Basic | | | |
|---|---|---|---|---|
| | immunisation: | 1. Booster | 2. Booster | 3. Booster |
| Horse | 23.Mai 2000 | 6.Juni 2000 | 21.Juni 2000 | 14.Juli 2000 |
| Daggy | 35 μg GG + | 35 μg GG + | 35 μg GG + | 35 μg GG + |
| | 1400 μg i.m | 1400 μg i.m | 1400 μg i.m | 1400 μg i.m |
| Frieda | 35 μg GG + | 35 μg GG + | 35 μg GG + | 35 μg GG + |
| | 1400 μg i.m | 1400 μg i.m | 1400 μg i.m | 1400 μg i.m |
| Friedrich | 35 μg GG + | 35 μg GG + | 35 μg GG + | 35 μg GG + |
| | 1400 μg i.m | 1400 μg i.m | 1400 μg i.m | 1400 μg i.m |
| Jessy | 35 μg GG + | 35 μg GG + | 35 μg GG + | 35 μg GG + |
| | 1400 μg i.m | 1400 μg i.m | 1400 μg i.m | 1400 μg i.m |
| Nelke | 35 μg GG + | 35 μg GG + | 35 μg GG + | 35 μg GG + |
| | 1400 μg i.m | 1400 μg i.m | 1400 μg i.m | 1400 μg i.m |

TABLE 10
Age of Horses

| | | anti-EAV |
|---|---|---|
| horses | age (years) | antibodies* |
| Daggi | 22 | ++ |
| Frieda | 16 | ? |
| Jessy | 16 | − |
| Friedrich | 10 | − |
| Nelke | 6 | − |

*before immunisation (10/99)

TABLE 11
Sample collection and immunization regimen

| | DNA | collection of | isolation |
|---|---|---|---|
| | application | plasma samples | of PBMC* |
| prae-immunisation | n.d. | 02.03.00 | 02.03.00 |
| prae-immunisation | n.d. | 23.03.00 | 23.03.00 |
| prae-immunisation | n.d. | 22.05.00 | 22.05.00 |
| Immunisation | 23.05.00 | 05.06.00 | 05.06.00 |
| 1. Booster | 06.06.00 | 20.06.00 | 20.06.00 |
| 2. Booster | 21.06.00 | 13.07.00 | 13.07.00 |
| 3. Booster | 14.07.00 | 28.07.00 | 28.07.00 |
| | n.d. | 25.08.00 | 25.08.00 |
| | n.d. | 22.09.00 | 22.09.00 |

*peripheral blood mononuclear cells
n.d.: not done

TABLE 12
Skin Reaktions of Horses: 24 hours post injectionem after first immunisation*

| | Daggy | | Frieda | | Nelke | | Friedrich | | Jessy | |
|---|---|---|---|---|---|---|---|---|---|---|
| Numbers of | le: 6 | | le: 4 | | le: 2 | | le: 5 | | le: 6 | |
| papules | ri: 6 | | ri. 6 | | ri: 1 | | ri: 5 | | ri: 6 | |
| Description of | le: severe swollen, | | le: severe swollen, | | le: moderate swollen, | | le: not to mild | | le: mild to moderate | |
| papules | epidermis becomes | | epidermis becomes | | Str.c. normal | | swollen, | | swollen, | |
| | eroded | | severe eroded | | ri: see left | | Str.c. normal | | Str.c. scaled | |
| | ri: severe swollen, | | ri: moderate swollen, | | | | ri: moderate swollen, | | ri: mild swollen, | |
| | 1 papule without | | Str.c. mild eroded | | | | Str.c. normal | | Str.c. scaled | |
| | epidermis | | | | | | | | | |
| Skin thickness | le: | ri: | le: | ri: | le: | ri: | le: | ri: | lc: | ri: |
| i) normal | 0.25 cm | 0.30 cm | 0.30 cm | 0.30 cm | 0.20 cm | 0.20 cm | 0.30 cm | 0.35 cm | 0.40 cm | 0.40 cm |
| ii) Papules | 0.60 cm | 0.60 cm | 0.60 cm | 0.60 cm | 0.45 cm | 0.40 cm | 0.40 cm | 0.40 cm | 0.60 cm | 0.60 cm |

| | | | | | |
|---|---|---|---|---|---|
| Diameter | le: 1.1-1.2 cm | le: 0.8-1.0 cm | le: 0.6-0.8 cm | le: 1.0-1.1 cm | le: 0.5-0.6 cm |
| | ri: 1.0 cm | ri: 0.7-0.8 cm | ri: 0.7 cm | ri: 0.9-1.2 cm | ri: 0.5 cm |
| | | | | | 1 papule: 1.0 cm |
| Features | | On both sides: | Very thin skin, | 1 papule: Str.c. | 1 papule with erosin, |
| | | gnat bites with | le: 2 white circles | mild swollen, congested | strong oedema and |
| | | allergic | | and folding of the | pruitus |
| | | oedema | | skin | |

le: left side of neck;
ri: right sight of neck;
Str.c.: Stratum corneum;
*Immunisation date: 23/05/2000; Skin reaction protocol date: 24/05/2000

TABLE 13
Skin Reaktions of Horses: 48 hours post injectionem after first immunisation*

| | Daggy | | Frieda | | Nelke | | Friedrich | | Jessy | |
|---|---|---|---|---|---|---|---|---|---|---|
| Numbers of | le: 6 | | le: 4 | | le: 2 | | le: 5 | | le: 6 | |
| papules | ri: 6 | | ri: 6 | | ri: 1 | | ri: 5 | | ri: 6 | |
| Description of | le: severe swollen, | | lc: severe swollen, | | le: moderate swollen, | | le: no to mild | | le: mild to moderate | |
| papules | epidermis becomes | | epidermis becomes | | Str.c. normal | | swollen, | | swollen, | |
| | eroded | | severe eroded | | ri: see left | | Str.c. normal | | Str.c. scaled | |
| | ri: severe swollen, | | ri: moderate swollen, | | | | ri: moderate swollen, | | ri: mild swollen, | |
| | 1 papule without | | Str.c. mild eroded | | | | Str.c. normal | | Str.c. scaled | |
| | epidermis | | | | | | | | | |
| Skin thickness | le: | ri: | le: | ri: | le: | ri: | le: | ri: | le: | ri: |
| i) normal | 0.25 cm | 030 cm | 0.30 cm | 0.30 cm | 0.20 cm | 0.20 cm | 0.30 cm | 0.35 cm | 0.40 cm | 0.40 cm |
| ii) Papules | 0.70 cm | 0.60 cm | 0.50-o.60 cm | 0.45 cm | 0.45 cm | 0.40 cm | 0.40 cm | 0.40 cm | 0.60 cm | 0.60 cm |

| | | | | | |
|---|---|---|---|---|---|
| Diameter | le: 1.1-1.2 cm | le: 0.8-1.0 cm | le: 0.6-0.8 cm | le: 1.0-1.1 cm | le: 0.5 |
| | ri: 1.0 cm | ri: 0.7-0.8 cm | ri: 0.7 cm | ri: 0.9-1.2 cm | ri: 0.9-1.2 cm |
| Features | | On both sides: | Very thin skin, | 1 papule: Str.c. | 1 papule with erosin, |
| | | gnat bites with | le: 2 white circles | mild swollen, | strong oedema and |
| | | allergic | | congested and | pruitus |
| | | oedema | | folding of the | |
| | | | | skin | |

le: left side of neck;
ri: right sight of neck;
Str.c.: Stratum corneum;
*Immunisation date: 23/05/2000; Skin reaction protocol date: 24/05/2000

### EXAMPLE 3

### Cytotoxic T-Lymphocyte (CTL Assay)

1. Immunization of Horses

1.1. DNA Application and Observed Skin Reactions

The horses (Daggy, Frieda, Friedrich, Jessy, Nelke) were immunised according to the immunisation regimen (Table 14) by intramuscular (i.m.) injection and intradermal gene gun application of EAV ORF-2, EAV ORF-5, and EAV ORF-7 expression plasmids. The i.m. inoculations were applied to the musculi semimembranosus/semitendinosus/gluteus. The gene gun applications of the DNA were performed on both sides of the neck. To enable an optimal gene gun DNA application the corresponding parts of the skin (40 cm²) had been shaved before. The horses were investigated 24 h after the DNA applications for systemic and local reactions. None of the animals developed fever or local reactions such as thickening of the skin, development and involution of papules. Protocols are included as Tables 12 and 13 (see above). Photographs of the skin areas involved were taken 24 h after DNA application.

TABLE 14
Sample collection and immunisation regimen

| | DNA | collection of | isolation |
|---|---|---|---|
| | application | plasma samples | of PBMC* |
| Prae-immunisation | n.d. | 02.03.00 | 02.03.00 |
| Prae-immunisation | n.d. | 23.03.00 | 23.03.00 |
| Prae-immunisation | n.d. | 22.05.00 | 22.05.00 |
| Immunisation | 23.05.00 | 05.06.00 | 05.06.00 |
| 1. Booster | 06.06.00 | 20.06.00 | 20.06.00 |
| 2. Booster | 21.06.00 | 13.07.00 | 13.07.00 |
| 3. Booster | 14.07.00 | 28.07.00 | 28.07.00 |

*peripheral blood mononuclear cells
n.d.: not done

1.2. Detection of EAV-Specific Antibodies

Serum and plasma samples of the immunised animals were taken two/three weeks after the DNA applciations as outlined in Table 14.

2. Preparation of Target Cells

2.1. Transfection Experiments in T25 Cell Culture Flasks

Skin fibroblasts (1.43×10⁶) cells) were incubated at 37° C. and 5% and 5% CO₂ in T25 (25 cm²) cell culture flasks using Dulbecco's modified Eagles medium (DMEM) containing 10% fetal calf serum (FCS) and antibiotics. Cells were transfected with 100 μg DNA of a Green Fluorescent Protein (GFP) expression plasmid in 1.3 ml Optimem® (Gibco) by the addition of 87.5 μl LipofectAMINE® (Gibco). After 24 h cells were treated with trypsin and seeded in a concentration of 5×10³ to 5×10⁴ cells per well in 96-well-plates. About 20% of the cells were transfected after 24 h of incubation at 37° C. and 5% CO₂ as determined by immunofluorescence.

2.2. Titration Experiments Using Polyclonal EAV-Specific Rabbit Serum (11.08.98)

Vero-cells (10⁴) were seeded in each well of a 96-well-plate and grown over night (o/n) in DMEM culture medium containing 10% FCS and antibiotics at 37° C. and 5% CO₂. The monolayers were subsequently infected with a 1:100 or 1:1000 diluted Vero-cell propagated EAV stock (Institute of Virology, University of Leipzig). The first EAV-specific cytopathic effects (CPE) were observed 36 h post infection and the cultures were immediately fixed for 30 min with ethanol at 4° C. The 96-well-plate was stored with ethanol at −70° C.

For immunohistochemical detection of viral antigens in infected cells, cultures were rinsed for 5 min with phosphate buffered saline (PBS) at room remperature (RT). In order to block endogenous peroxidases, cultures were incubated for 5 min with 7.5% H₂O₂ in methanol. Cells were rinsed with PBS and incubated for 1 h with serum dilutions (polyclonal EAV-specific rabbit serum from 11.08.98, pre-immune [rabbit #98/8] from 10.06.98 ranging from 1:100 and 1:200 up to 1:1600 in 0.05% Tween 20-PBS. Cultures were rinsed twice with 0.05% Tween 20-PBS and once with destined water and incubated for 1 h with a biotinylated anti-rabbit antibody diluted 1:750 at 37° C. Cells were rinsed as before with Tween 20-PBS and destined water and incubated for 30 min at 37° C. with streptavidin-peroxidase (1:500 in Tween 20-PBS). Cultures were rinsed again and the substrate AEC (14.25 ml Na-Acetat buffer 0.75 μl AEC, 75 μl H₂O₂ 1:10 diluted in destined water) was added. Even in the highest antibody dilution (1:1600) clear positive reactions were visible.

2.3. Transfection of Skin Fibroblasts with EAV-Specific Expression Plasmids

Transfection experiments were performed similarly to the protocol described in example 2 using the 24-well-plate format. Immunohistochemical analysis using the polyclonal EAV-specific rabbit serum (11.08.98), diluted 1:800 in 0.05% Tween 20-PBS, showed that about 20% of the cells were transfected with the EAV ORF-2, EAV ORF-5, and EAV ORF-7 expression plasmids.

2.4. Culturing of Target Cells Prior to CTL Assay

Skin fibroblasts of the horses were cultured in Iscove's modified Eagles medium (IMEM) containing 10% FCS and antibiotics. Cultures were extended to three T25 cell culture flasks and transfected when being confluent for 80-90% with each 100 μg of the EAV ORF-2, EAV ORF-5, and EAV ORF-7 expression plasmids. After 24 h, cultures were treated with trypsin, cells were counted and labeled in suspension with ⁵¹Cr.

3. Preparation of Effector Cells

3.1. Inactivation of EAV Using ¹³⁷Cs γ-Rays

In order to obtain viral antigen for the restimulation of EAV-specific effector cells, Vero-cells were infected with EAV at a multiplicity of infection (moi) of 1. After 36 h, cell cultures were freeze/thawed two times and the cultures were centrifuged for 5 min at 1750×g. The supernatant was pelleted by centrifugation o/n at 19.000 RPM. The pellet was resuspended in 3 ml PBS and the protein concentration was measured using a BCA-Protein Assay® (Pierce). Aliquots of the samples containing 2 mg/ml were stored at −70° C.

Samples were exposed on dry ice to ¹³⁷Cs γ-rays ranging from 15 Gy up to 1 k Gy. Virus titration on Vero-cells revealed that the infectious titre had decreased only by <log1, even after exposure to 1 k Gy.

3.2 Inactivation of EAV Using Ultraviolet Radiation

Because EAV could not be inactivated by ¹³⁷CS γ-rays, samples were exposed to ultraviolet radiation (254 nm) at a distance of 5 cm for 5 min up to 80 min. Virus titration on Vero-cells revealed that no infectious virus could be detected after a 5 min exposure. To completely inactivate EAV in the samples used for the restimulation of the effector cells, these samples were exposed for 30 min and stored aliquots containing 2 mg of protein per ml at −70° C.

3.2. Culturing of Effector Cells Prior to CTL Assay

PBMC were cultured in IMEM containing 10% FCS and antibiotics. After stimulation for two days with 2.5 μg/ml pookweed mitogen, cells were cultured for two days in the presence of 200 U of human recombinant IL-2 (Amersham-Pharmacia). EAV-specific cytotoxic T-cells were restimulated for four days with 30 μg/ml inactivated EAV in the presence of 100 U IL-2. Thereafter, cultures were extended for four days in the presence of 200 U of IL-2. Cells were counted and used in the CTL assay.

4. Measurement of EAV-Specific Cytotoxic T-Cells

4.1. ⁵¹Cr-Labeling of Target Cells

After trypsin treatment, transfected target cells were resuspended in 1.5 ml and labeled for 2 h with 100 μCi ⁵¹Cr/10⁷ cells. Cells were washed three times with culture medium and 7.7×10³ cells were seeded in each well of a 96 U-bottom cell culture plate in a volume of 150 μl. Cultures were incubated for 4 h at 37° C. and 5% CO₂ to let cells adhere to the plate.

4.2. Addition of Effector Cells

PBMC were counted and added to the cultures in 100 μl volumes and effector/target cell ratios of 3:1, 25:1, and 50:1. Cells were incubated for 8 h in a total volume of 250 μl at 37° C. and 5% CO₂.

4.3. Measurement of ⁵¹Cr Release in Cultures with Different Effector/Target Cell Ratios

Culture plates were centrifuged at 1000 RPM for 3 min and each supernatant was measured for the presence of ⁵¹Cr in a scintillation counter for 60 sec. Negative control culture supernatants consisted of labeled cells without the addition of effector cells (spontaneous ⁵¹Cr release of the cells). Positive control culture supernatants consisted of cultures of labeled cells after cell lysis using 10% Triton X-100 (maximum ⁵¹Cr uptake). The results of the ⁵¹Cr release of the individual cultures including the effector cells prior to immunisation, two weeks after the 2nd immunisation, and two weeks after the 3^{rd} booster immunisation (Table 14) are included s. table 18. FIGS. 9a-e) show the calculated specific lysis ≧0 according to Hammond S A, Issel C J, and Montelaro R C (1998): General method for the detection and in vitro expansion of equine cytolytic T lymphocytes. J. Immunol Methods 213: 73-85.

The data sheets of the ⁵¹Cr release of the individual cultures show a relative small difference between the negative control culture supernatants (spontaneous ⁵¹Cr release of the cells) and the positive control culture supernatants (Maximum ⁵¹Cr uptake by the cells). The specific lysis is therefore difficult to measure, even if the standard deviations between the four individually handled cultures of each sample are small (Table 18).

### EXAMPLE 4

### DNA-Vaccination of Horses

1. Schedule for DNA for Vaccination of Horses

| | | |
|---|---|---|
| May 23, 2000 | 1^{st} immunization | day 0 |
| Jun. 6, 2000 | 2^{nd} immunization | day 14 |
| Jun. 21, 2000 | 3^{rd} immunization | day 29 |
| Jul. 14, 2000 | 4^{th} immunization | day 51 |

2. Strategy and Preparation of DNA for Vaccination
2.1. Application of DNA using Helios Gene Gun System (BIO-RAD):
2.1.1. Number of shots per animal and per vaccination: 10
2.1.2. Total DNA per shot: 3.5 μg

2.1.3. Number of expression vectors used: 7 (the properties and source of the DNA used for vaccination are summarized in Table 15)

| | |
|---|---|
| 1. | pCR3.1-EAV-O2-BX-C5 |
| 2. | pCR3.1-EAV-O5-BX-C14 |
| 3. | pDP-EAV-O5-BgS-C1 |
| 4. | pCR3.1-HIS-EAV-O5del-121-C12 |
| 5. | pCR3.1-EAV-O7-BX-C3 |
| 6. | pDP-EAV-O7-BgS-C2 |
| 7. | pCR3.1-Horse-IL2 |

2.1.4. DNA of individual expression vector per shot: 0.5 μg=500 ng
2.1.5. Total DNA of individual expression vector per animal and per vaccination: 5 μg
2.1.6. Total DNA per vaccination per animal: 7×5=35 μg
2.1.7. Number of animals: 5
2.1.8. Total DNA of individual expression vector per vaccination: 5×35=175 μg
2.1.9. Number of vaccination: 4
2.1.10. Total DNA of individual expression vector administered: 175×4=700 μg
2.2. Application of DNA through intramuscular route.
2.2.1. Number of inoculations per animal and per vaccination: 4
2.2.2. DNA of individual expression vector per inoculation: 50 μg
2.2.3. Total DNA of individual expression vector per animal and per vaccination: 50×4=200 μg
2.2.4. Number of expression vectors used: 7 (see Table 15)
2.2.5. Total DNA per vaccination per animal: 7×200=1.4 mg
2.2.6. Number of animals: 5
2.2.7. Total DNA of expression vector administered: 5×1.4=7.0 mg
2.2.8. Number of vaccination: 4
2.2.9. Total DNA of individual expression vector administered: 200×5×4=4 mg
2.2.10. Transfection reagent:
   DOTAP Liposomal (Roche; Cat.# 1811177) 50 μg/ml BME (3 ml/animal)
   Buffer I containing DNA (yellow cup tube)
Lipofectin (Life Technology; Cat.# 18292-011) 50 μg/ml BME (3 ml/animal)

Buffer II (white cup tube) add to DNA prior to application

TABLE 15
Properties of the cDNA used for the
undividual DNA-vaccination of horses

DNA used per
vaccination by
μg/horse

| | Gene | Classical |
|---|---|---|
| Expression vectors | GUN | i.m. |
| PCR3.1-EAV-O2-BX-C5 (1 μg/μl)* | 5 | 200 |
| 1.clone/5. Passage 16.06.1999 | | |
| Gene product: small glycoprotein | | |
| pCR3.1-EAV-O3-BX-C1 (1 μg/μl)* | | |
| 1.clone/6. Passage 17.06.1999 | | |
| Gene product: unknown | | |
| pCR3.1-EAV-O4-BX-C3 (1 μg/μl)* | | |
| 1.clone/6. Passage 18.06.1999 | | |
| Gene product: unknown | | |
| pCR3.1-EAV-O5-BX-C14 (1 μg/μl) | 5 | 200 |
| 1.clone/5. Passage 05.06.1999 | | |
| pDP-EAV-O5-BgS-C1 (1 μg/μl) | 5 | 200 |
| 1.clone/5. Passage 07.06.1999 | | |
| pCR3.1-HIS-EAV-O5del-121-C12 (1 μg/μl) | 5 | 200 |
| 1.clone/6. Passage 04.06.1999 | | |
| Gene product: large envelope | | |
| glycoprotein | | |
| pCR3.1-EAV-O6-BE-C4 ( 1 μg/μl) | | |
| 1.clone/5. Passage 15.06.1999 | | |
| Gene product: membrane protein | | |
| pCR3.1-EAV-O7-BX-C3 (1 μg/μl) | 5 | 200 |
| 1.clone/6. Passage 09.06.1999 | | |
| pDP-EAV-O7-BgS-C2 (1 μg/μl) | 5 | 200 |
| 1.clone/5. Passage 10.06.1999 | | |
| Gene product: nucleocapsid protein | | |
| pCR3.1-Horse-IL2 (1 μg/2 μl) | 5 | 200 |
| 2.clone/2. Passage 06.02.2000 | | |
| Gene product: Horse interleukin-2 | | |

3. Preparation of the DNA for Vaccination of Horses Using Helios Gene Gun System (BIO-RAD) and Optimization Kit (Catalog 165-2424)
3.1. Calculation:
3.1.1. MLQ=1 (1 mg Au/shot)
3.1.2. DRL=3.5 (3.5 μg DNA/mg Au, particle=1.6 μm)
3.1.3. Cartridge length=13 mm
3.1.4. Nalgene tubing=750 mm
3.1.5. Factor for Au=750/13=58 mg Au
3.1.6. 58×3.5 μg DNA=203 μg DNA ˜210 μg DNA
3.2. Procedure:
3.2.1. In a 1.5 ml microfuge tube, weigh out 60 mg Au.
3.2.2. To the measured Au add 210 μl 0.05 M Spermidine.
3.2.3. Vortex # 3.2.2. for few seconds, then sonicate 3-5 seconds.
3.2.4. To the Au and Spermidine mixture, add 210 μg total DNA of seven expression vectors=30 μg of individual expression vectors).
3.2.5. Mix DNA, Spermidine and Au by vortexing ˜5 seconds.
3.2.6. Add 210 μL CaCl₂ dropwise into # 3.2.5. while vortexing at a moderate rate
3.2.7. Precipitate for 10 min in room temperature.
3.2.8. Most of the Au will now be in a pellet, but some may be on the sides of the tube.

The supernatant should be relatively clear. Spin the microcarrier solution in a microfuge for 15 seconds to pellet the Au.
3.2.9. Remove the supernatant and discard.
3.2.10. Resuspend the pellet in the remaining supernatant by vortexing briefly. Wash the pellet three times with 1 ml 100% ethanol each time.
3.2.11. Spin for 5 seconds in a microfuge between each wash. Discard the supernatants.
3.2.12. After the final ethanol wash, resuspend the pellet in 200 μl of the ethanol solution containing 0.05 mg/ml PVP in ethanol.
3.2.13. Add # 3.2.12 in 2800 μl 0.05 mg/ml PVP in ethanol.
3.2.14. Preparation of cartridges:

May 20, 2000: 300 cartridges were prepared according to the above protocol and the Helios Gene Gun system Instruction Manual. Each cartridge=single shot contains 3.5 μg DNA corresponding to 0.5 μg=500 ng DNA of individual expression vectors.

4. Preimmune Sera

The preimmune sera of 5 horses (Daggi, Frieda, Friedrich, Jessy, and Nelke) that were obtained in October 1999 and analysed by NT and ELISA tests developed in our laboratory for screening of antibodies against equine arteritis virus (EAV). The results are summarized in Table 16.

TABLE 16
The results of neutralization (NT) and ELISA tests in which
the preimmune sera of 5 horses were screened for existence
of a humoral immune response developed during a natural infection
with wild type equine arteritis virus (EAV)

| | | Titre of serum | Titre of serum | |
|---|---|---|---|---|
| No | Horse | NT^{a} | ELISA | Remarks |
| 1 | Daggi | 1:40 | 1:100 | positive |
| 2 | Frieda | 1:20 | 1:50 | boundary |
| 3 | Friedrich | 1:10 | <1:50 | |
| 4 | Jessy | 1:10 | <1:50 | |
| 5 | Nelke | 1:10 | <1:50 | |
| | Positive | 1:160^{b} | 1:200 | positive |
| | Control | 1:80^{c} | | positive |
| | Serum | | | |

^{a}Multiplicity of infection (MOI): 100 PFU of EAV/1000 RK-13 cells/well
^{b}Rabbit antiserum raised against EAV
^{c}Horse serum was obtained from Prof. Dr. Ludwig, Berlin

Vaccination protocol
Tuesday, May 23, 2000
1. Race of horses: Jessy: Shetland Pony, Rest: so called “Warm-blooder”

2. Age of horses

| | | | |
|---|---|---|---|
| 2.1. | Daggi: | 22 | years old |
| 3.2. | Frieda: | 16 | years old |
| 3.3. | Friedrich: | 10 | years old |
| 3.4. | Jessy: | 16 | years old |
| 3.5. | Nelke: | 6 | years old |

3. Vaccination:

| | | | | | | |
|---|---|---|---|---|---|---|
| 3.1. | Jessy | start | 12.15 | end | 12.22 | (10 × shot: |
| | | | | | | 5 per neck side) |
| 3.2. | Nelke | start | 12.30 * | end | 12.38 | (10 × shot: |
| | | | | | | 5 per neck side) |
| 3.3. | Daggi | start | 12.45 §& | end | 12.55 | (12 × shot: |
| | | | | | | 6 per neck side) |
| 3.4. | Frieda | start | 12.47 §& | end | 12.57 | (12 × shot: |
| | | | | | | 6 per neck side) |
| 3.5. | Friedrich | start | 13.10 § | end | 13.15 | (12 × shot: |
| | | | | | | 6 per neck side) |

* Sedativum: Domosedan (Pfizer) = Methyl-4-hydrooxybenzoad 1 ml i.v. (10 mg)
§ Rometar (Serum-Werk Bernburg) = Xylazin i.v. Methyl-4-hydrooxybenzoad
& Daggi and Frieda had been vaccinated together

& Daggi and Frieda had been vaccinated together
4. Sera: 2^{nd} preimmune serum was taken on Monday, May 22, 2000 (3 ml per animal)
5. May 24, 2000: All vaccinated horses developed skin reactions “DTH-reaction” at the target of the shot.
Tuesday, Jun. 6, 2000
6. Sera: 1^{st} post vaccinated serum was taken on Monday, Jun. 5, 2000 (3 ml per animal)

7. Vaccination:

| | | | | | | |
|---|---|---|---|---|---|---|
| 3.1. | Frieda | start | 12.25* | end | 12.30 | (10 × shot: |
| | | | | | | 5 per neck side) |
| 3.2. | Daggi | start | 12.27* | end | 12.33 | (10 × shot: |
| | | | | | | 5 per neck side) |
| 3.3. | Jessy | start | 12.37* | end | 12.43 | (10 × shot: |
| | | | | | | 5 per neck side) |
| 3.4. | Nelke | start | 12.44* | end | 12.47 | (10 × shot: |
| | | | | | | 5 per neck side) |
| 3.5. | Friedrich | start | 12.49* | end | 12.54 | (10 × shot: |
| | | | | | | 5 per neck side) |

*Sedativum: Rometar = Xylazin i.v. Methyl-4 hydrooxybenzoad 2% (Serum-Werk Bernburg)

Vaccination protocol
Tuesday, Jun. 21, 2000
8. Sera: 2^{nd} post vaccinated serum was taken on Monday, Jun. 20, 2000 (1.5 ml per animal)

9. Vaccination:

| | | | | | | |
|---|---|---|---|---|---|---|
| 3.1. | Frieda | start | 12.10* | end | 12.16 | (12 × shot: |
| | | | | | | 6 per neck side) |
| 3.2. | Daggi | start | 12.17* | end | 12.20 | (12 × shot: |
| | | | | | | 6 per neck side) |
| 3.3. | Jessy | start | 12.25* | end | 12.28 | (12 × shot: |
| | | | | | | 6 per neck side) |
| 3.4. | Nelke | start | 12.29* | end | 12.32 | (11 × shot: |
| | | | | | | 5 & 6 per neck side) |
| 3.5. | Friedrich | start | 12.36* | end | 12.39 | (12 × shot: |
| | | | | | | 6 per neck side) |

*Sedativum: Rometar = Xylazin i.v. Methyl-4 hydrooxybenzoad 2% (Serum-Werk Bernburg)

Vaccination protocol
Friday, Jul. 14, 2000
10. Sera: 3^{rd} post vaccinated serum was taken on Jul. 13, 2000, 1.5 ml per animal

11. Vaccination:

| | | | | | | |
|---|---|---|---|---|---|---|
| 3.1. | Daggi | start | 12.00* | end | 12.07 | (12 × shot: |
| | | | | | | 6 per neck side, 530 psi) |
| 3.2. | Frieda | start | 12.08* | end | 12.11 | (12 × shot: |
| | | | | | | 6 per neck side, 530 psi) |
| 3.3. | Jessy | start | 12.21* | end | 12.24 | (12 × shot: |
| | | | | | | 6 per neck side, 430 psi) |
| 3.4. | Nelke | start | 12.25* | end | 12.27 | (12 × shot: |
| | | | | | | 6 per neck side, 500 psi) |
| 3.5. | Friedrich | start | 12.35* | end | 12.42 | (12 × shot: |
| | | | | | | 6 per neck side, 400 psi) |

*Sedativum: Domosedan (Pfizer) = Methyl-4-hydrooxybenzoad 1 ml i.v. (10 mg) § Rometar (Serum-Werk Bernburg) = Xylazin i.v. Methyl-4-hydrooxybenzoad

12. Sera: 4^{th} post vaccinated serum was taken on Jul. 28, 2000, 1.5 ml per animal
13. Determination of neutralizing antibody.

The determination of neutralizing antibodies of individual horse sera (30 serum samples that was labelled with code number 1-30, see Table 3) was performed. The results of neutralization tests are summarized in Table 17.

TABLE 17
The results of neutralization tests (NT) in which the sera of five horses were screened
for detection of a humoral immune response developed after DNA vaccination with the cDNA
of ORFs 2, 5, and 7 of equine arteritis virus (EAV) as indicated in Table 15 and 16

| | NT-Titre of individual horses taken at the different times | | | | | |
|---|---|---|---|---|---|---|
| | prior and post DNA-Vaccination^{a} | | | | | |
| | | | post | post | post | post |
| | Preimmune | preimmune | vaccinated | vaccinated | vaccinated | vaccinated |
| | serum 1 | serum 2 | serum 1 | serum 2 | serum 3 | serum 4 |
| Horse | 20.10.1999 | 22.05.2000 | 05.06.2000 | 20.06.2000 | 13.07.2000 | 28.07.2000 |
| 1. | 1:16 (#01) | 1:64 | 1:256 | 1:256 | 1:256 | 1:256 |
| Daggi | | (#06) | (#11) | (#16) | (#21) | (#26) |
| | | | 1:256^{e} | | 1:256^{e} | |
| 2. | <1:2 (#02) | <1:2 | 1:256 | 1:256 | 1:128 | 1:256 |
| Frieda | | (#07) | (#12) | (#17) | (#22) | (#27) |
| | | | 1:256^{e} | 1:256^{e} | | 1:256^{e} |
| 2. | <1:2 (#03) | <1:2 | 1:64 | 1:128 | 1:128 | 1:128 |
| Friedrich | | (#08) | (#13) | (#18) | (#23) | (#28) |
| 4. | <1:2 (#04) | <1:2 | 1:256 | 1:256 | 1:256 | 1:128 |
| Jessy | | (#09) | (#14) | (#19) | (#24) | (#29) |
| | | | 1:256^{e} | 1:256^{e} | 1:256^{e} | |
| 5. | <1:2 (#05) | 1:8 | 1:256 | 1:128 | 1:256 | <1:2^{C} |
| Nelke | | (#10) | (#15) | (#20) | (#25) | (#30) |
| | | | 1:512 | | 1:512^{e} | l:256^{d} |
| | | | (#15) | | | |

^{a}DNA-Vaccination of horses was performed as follows: May 23, 2000: 1^{st} immunization (day 0), Jun. 6, 2000: 2^{nd} immunization (day 14),
Jun. 21, 2000: 3^{rd} immunization (day 29), and Jul. 14, 2000: 4^{th} immunization (day 51)
^{b}The number in parenthesis indicate the code number of individual sera used by
neutralization test, that was performed by Dr. Herzog, Veterinary laboratory at the
Institute for clinical analysis, 71611 Ludwigsburg, Germany.
^{C}This titre has to be checked again.
^{d}The titre 1:2 was a type mistake. The correct titre was found to be 1:256, (Dr. Herzog laboratory, 08/20/2000)
^{e}The results of the second analysis obtained from those sera with a titre of 1:256, (Dr. Herzog laboratory, 09/06/2000)

Biological material
14. Sera: 5^{th} post vaccinated serum was taken on Aug. 25, 2000 (2×1.5 ml per animal)
15. Sera: 6^{th} post vaccinated serum was taken on Sep. 22, 2000 (2×1.5 ml per animal)

16. Sera: 7^{th} post vaccinated serum was taken on Oct. 23, 2000 (2×1.5 ml per animal).

TABLE 18a
Example 5
Table 18: ⁵¹Cr-release of the individual cultures
2. CTL-Assay, 10./11.08.2000

| | | | | | Prior to Immunization | | |
|---|---|---|---|---|---|---|---|
| | | | | | E:T | | |
| Horse | Cells | ORF | Negative Control | Positive Control | 3 to 1 | 25 to 1 | 50 to 1 |
| Daggy | Transfected | 2 | | | | | |
| | | | 337 | 791 | 448 | 521 | 710 |
| | | | 381 | 615 | 495 | 681 | 525 |
| | | | 392 | 658 | 540 | 389 | 509 |
| | | | 360 | 952 | 542 | 558 | 457 |
| | | Mean | 382.5 | 854 | 508.5 | 532.25 | 551 |
| | | SD | 22.5757883 | 70.869477 | 43.921929 | 112.378453 | 110.166541 |
| | | Specif. Lysis | | | 0.20784985 | 0.34537131 | 0.38351934 |
| Daggy | Transfected | 5 | 211 | 499 | | | |
| | | | 252 | 594 | | | |
| | | | 243 | 556 | | | |
| | | | 221 | 598 | | | |
| | | | | | 250 | 255 | 207 |
| | | | 255 | 501 | 277 | 277 | 330 |
| | | | 270 | 482 | 285 | 296 | 330 |
| | | | 238 | 637 | 225 | 282 | 230 |
| | | Mean | 241.142857 | 549.571429 | 261.75 | 277.5 | 299.25 |
| | | SD | 20.3588883 | 84.0178547 | 30.596891 | 17.0195065 | 44.3198601 |
| | | Specif. Lysis | | | 9.06681334 | 0.15767885 | 0.16515516 |
| Daggy | Transfected | 7 | | | | | |
| | | | 350 | 542 | 812 | 449 | 450 |
| | | | 308 | 719 | 564 | 551 | 445 |
| | | | 261 | 809 | 452 | 445 | 382 |
| | | | 270 | 578 | 577 | 435 | 385 |
| | | Mean | 297.26 | 811.5 | 553.75 | 470.5 | 410.5 |
| | | SD | 40.8395780 | 76.7094084 | 64.4379547 | 65.3142537 | 43.7911711 |
| | | Specif. Lysis | | | 0.51622912 | 0.65151265 | 0.36038158 |

| | | | After the 2nd Booster | | | After the 3rd Booster | | |
|---|---|---|---|---|---|---|---|---|
| | | | E:T | | | E:T | | |
| Horse | Cells | ORF | 3 to 1 | 25 to 1 | 50 to 1 | 3 to 1 | 25 to 1 | 50 to 1 |
| Dag- | Trans- | 2 | | | | | | |
| gy | fected | | | | | | | |
| | | | 503 | 444 | 312 | 556 | 594 | |
| | | | 473 | 512 | 417 | 400 | 640 | |
| | | | 600 | 481 | 402 | 668 | 541 | |
| | | | 709 | 518 | 407 | 416 | 730 | 791 |
| | | Mean | 548.25 | 486.25 | 384.5 | 628.75 | 650 | 791 |
| | | SD | 109.335493 | 33.3903879 | 48.7339717 | 106.019874 | 139.63972 | #DIV:01 |
| | | Specif. | 0.37385534 | 0.25584944 | 0.04476094 | 0.34928481 | 0.64586169 | 0.87182098 |
| | | Lysis | | | | | | |
| Dag- | Trans- | 5 | | | | | | |
| gy | fected | | | | | | | |
| | | | 192 | 259 | 263 | 226 | 233 | |
| | | | 220 | 288 | 259 | 232 | 280 | 247 |
| | | | 223 | 254 | 208 | 242 | 279 | 252 |
| | | | 244 | 274 | 301 | 200 | 223 | 247 |
| | | Mean | 221.75 | 270.75 | 280.25 | 225 | 248.75 | 248.586687 |
| | | SD | 21.7619689 | 11.9286604 | 22.3215143 | 17.8257729 | 25.5130685 | 2.68875135 |
| | | Specif. | −0.06287633 | 0.09590352 | 0.12679451 | −0.05233905 | 0.0246842 | 8.02439401 |
| | | Lysis | | | | | | |
| Dag- | Trans- | 7 | | | | | | |
| gy | fected | | | | | | | |
| | | | 405 | 493 | 407 | 271 | 249 | 251 |
| | | | 383 | 499 | 439 | 206 | 298 | 350 |
| | | | 453 | 630 | 391 | 241 | 265 | 464 |
| | | | 368 | 425 | 430 | 301 | 297 | 493 |
| | | Mean | 417.5 | 512.5 | 416.75 | 277.25 | 275.25 | 394.5 |
| | | SD | 49.3254496 | 84.6738542 | 21.0231526 | 27.5 | 22.2407226 | 115.689006 |
| | | Specif. | 0.38265712 | 0.6849642 | 0.38027049 | −0.0638436 | −0.07000798 | 0.30048098 |
| | | Lysis | | | | | | |

TABLE 18b
2. CTL-Assay, 10./11.08.2000

Prior to Immunization
E:T

| Horse | Cells | ORF | Negative Control | Positive Control | 3 to 1 | 25 to 1 | 50 to 1 |
|---|---|---|---|---|---|---|---|
| Jessy | Transfected | 2 | 516 | 404 | | | |
| | | | 476 | 514 | | | |
| | | | 468 | 446 | | | |
| | | | 381 | 477 | | | |
| | | | | 1080 | 687 | 526 | 538 |
| | | | | 1082 | 570 | 889 | 530 |
| | | | | 809 | 446 | 513 | 577 |
| | | | | 1235 | 646 | 542 | 458 |
| | | Mean | 460.25 | 763.125 | 557.25 | 538.5 | 821.5 |
| | | SD | 56.8528803 | 339.304836 | 80.7574907 | 83.8431074 | 62.8384768 |
| | | Specif. Lysis | | | 0.31712301 | 0.35390274 | 0.2002452 |
| Jessy | Transfected | 5 | 225 | 309 | | | |
| | | | 257 | 246 | | | |
| | | | 229 | 404 | | | |
| | | | 242 | 334 | | | |
| | | | 167 | 353 | 255 | 251 | 246 |
| | | | 217 | 286 | 265 | 257 | 266 |
| | | | 216 | 280 | 236 | 276 | 244 |
| | | | 220 | 286 | 249 | 276 | 256 |
| | | Mean | 224.125 | 308.9 | 250.26 | 254.75 | 255.6 |
| | | SD | 20.4950538 | 54.2428402 | 11.1768125 | 24.4712530 | 10.5039875 |
| | | Specif. Lysis | | | 0.51714719 | 0.37177542 | 0.35280098 |
| Jessy | Transfected | 7 | 470 | 508 | | | |
| | | | 485 | 509 | | | |
| | | | 480 | 584 | | | |
| | | | 433 | 780 | | | |
| | | | 495 | 639 | 849 | 536 | 546 |
| | | | 517 | 571 | 820 | 588 | 652 |
| | | | 801 | 758 | 585 | 518 | 820 |
| | | | 777 | 1582 | 692 | 593 | 533 |
| | | Mean | 554.825 | 739 | 585.5 | 558 | 587.75 |
| | | SD | 146.882514 | 358.058124 | 46.9859746 | 97.2111095 | 57.4716072 |
| | | Specif. Lysis | | | 0.22189492 | 0.02972805 | 0.17989102 |

| | | | After the 2nd Booster | | | After the 3rd Booster | | |
|---|---|---|---|---|---|---|---|---|
| | | | E:T | | | E:T | | |
| Horse | Cells | ORF | 3 to 1 | 25 to 1 | 50 to 1 | 3 to 1 | 25 to 1 | 50 to 1 |
| Jes- | Trans- | 2 | | | | | | |
| sy | fected | | | | | | | |
| | | | 423 | 462 | 518 | 384 | 302 | 400 |
| | | | 526 | 526 | 497 | 448 | 396 | 484 |
| | | | 435 | 435 | 526 | 495 | 395 | 570 |
| | | | 434 | 434 | 508 | 422 | 424 | 363 |
| | | Mean | 449.6 | 501 | 512.75 | 437.25 | 379.25 | 465.5 |
| | | SD | 61.7204022 | 34.5000381 | 13.3010025 | 40.8145542 | 53.2251507 | 96.0858318 |
| | | Specif. | −0.03584508 | 0.13522436 | 0.17103874 | −0.07510812 | −0.28481406 | −0.01225991 |
| | | Lysis | | | | | | |
| Jes- | Trans- | 5 | | | | | | |
| sy | fected | | | | | | | |
| | | | 218 | 287 | 312 | 246 | 247 | 271 |
| | | | 280 | 274 | 293 | 219 | 252 | 289 |
| | | | 240 | 292 | 301 | 271 | 266 | 281 |
| | | | 253 | 274 | 291 | 260 | 267 | 308 |
| | | Mean | 242.25 | 291.75 | 299.25 | 247 | 252.5 | 287.25 |
| | | SD | 10.382755 | 8.17577988 | 9.53502318 | 21.3697608 | 10.214369 | 15.6710987 |
| | | Specif. | 0.22033055 | 0.59954476 | 0.81195766 | 0.27769347 | 0.34446131 | 0.75831250 |
| | | Lysis | | | | | | |
| Jes- | Trans- | 7 | | | | | | |
| sy | fected | | | | | | | |
| | | | 593 | 624 | 577 | 388 | 343 | 401 |
| | | | 489 | 571 | 805 | 409 | 407 | 423 |
| | | | 482 | 567 | 531 | 429 | 458 | 375 |
| | | | 515 | 607 | 563 | 381 | 398 | 403 |
| | | Mean | 800.75 | 592.29 | 589 | 398.75 | 401.5 | 480.5 |
| | | SD | 15.9861080 | 27.7773869 | 30.7678487 | 18.5898718 | 47.1082829 | 10.6992526 |
| | | Specif. | −0.29220530 | 0.2040670 | 0.0778561 | −8.80542373 | 0.83050847 | −8.8359322 |
| | | Lysis | | | | | | |

TABLE 18c
2. CTL-Assay, 10./11.08.2000

| | | | | | Prior to Immunization | | |
|---|---|---|---|---|---|---|---|
| | | | | | E:T | | |
| Horse | Cells | ORF | Negative Control | Positive Control | 3 to 1 | 25 to 1 | 50 to 1 |
| Friedrich | Transfected | 2 | 248 | 2103 | | | |
| | | | 259 | 2704 | | | |
| | | | 229 | 1633 | | | |
| | | | 248 | 2325 | | | |
| | | | 220 | 657 | 315 | 327 | 420 |
| | | | 314 | 657 | 312 | 274 | 291 |
| | | | | 637 | 299 | 269 | 273 |
| | | | 289 | 830 | 301 | 283 | 282 |
| | | Mean | 257.857143 | 1454 | 306.75 | 283.25 | 318 |
| | | SD | 83.2838605 | 882.106545 | 7.93200289 | 23.3291683 | 72.9740285 |
| | | Specif. Lysis | | | 0.04087543 | 0.02058916 | 0.05028066 |
| Friedrich | Transfected | 5 | 272 | 630 | | | |
| | | | 354 | 716 | | | |
| | | | 304 | 607 | | | |
| | | | 281 | 675 | | | |
| | | | 233 | 687 | 250 | 327 | 271 |
| | | | 270 | 632 | 283 | 279 | 256 |
| | | | 220 | 712 | 293 | 275 | 268 |
| | | | 242 | 730 | 259 | 319 | 269 |
| | | Mean | 272 | 599.75 | 287 | 380.75 | 288 |
| | | SD | 43.0248097 | 68.5485211 | 20.0831604 | 25.9020591 | 7.383691 |
| | | Specif. Lysis | | | −0.01188907 | 0.06721216 | −0.01402068 |
| Friederich | Transfected | 7 | 290 | 575 | | | |
| | | | 208 | 658 | | | |
| | | | 318 | 675 | | | |
| | | | 250 | 617 | | | |
| | | | 501 | 989 | 447 | 457 | 580 |
| | | | 537 | 1008 | 414 | 460 | 535 |
| | | | 389 | 1058 | 403 | 383 | 465 |
| | | | 424 | 989 | 436 | 331 | 590 |
| | | Mean | 375.375 | 811.25 | 425 | 407.75 | 547.6 |
| | | SD | 105.247922 | 225.178501 | 20.0831804 | 62.3397917 | 58.83565382 |
| | | Specif. Lysis | | | 0.11385145 | 0.07427588 | 0.30342415 |

| | | | After the 2nd Booster | | | After the 3rd Booster | | |
|---|---|---|---|---|---|---|---|---|
| | | | E:T | | | E:T | | |
| Horse | Cells | ORF | 3 to 1 | 25 to 1 | 50 to 1 | 3 to 1 | 25 to 1 | 50 to 1 |
| Fried- | Trans- | 2 | | | | | | |
| rich | fected | | | | | | | |
| | | | 245 | 289 | 247 | 318 | 228 | 814 |
| | | | 281 | 508 | 321 | 371 | 315 | 670 |
| | | | 267 | 693 | 267 | 335 | 353 | 749 |
| | | | 331 | 295 | 279 | 313 | 238 | 953 |
| | | Mean | 281 | 446.75 | 278.5 | 934.25 | 288 | 798.5 |
| | | SD | 36.4783041 | 198.858446 | 31.2560992 | 26.2472221 | 54.3894401 | 117.016941 |
| | | Specif. | 0.0103479 | 0.15959005 | 0.01725765 | 0.083853 | 0.02352601 | 0.45186653 |
| | | Lysis | | | | | | |
| Fried- | Trans- | 5 | | | | | | |
| rich | fected | | | | | | | |
| | | | 253 | 289 | 253 | 246 | 239 | 239 |
| | | | 277 | 230 | 348 | 238 | 290 | 230 |
| | | | 283 | 252 | 820 | 245 | 282 | 258 |
| | | | 263 | 202 | 343 | 277 | 295 | 286 |
| | | Mean | 259 | 275.25 | 315.5 | 251 | 276.5 | 255.25 |
| | | SD | 13.56496 | 18.4839288 | 43.2560575 | 17.907188 | 25.5659057 | 22.4703505 |
| | | Specif. | −0.00701344 | 0.0075979 | 0.10169492 | −0.0480841 | 0.01052018 | −0.03915839 |
| | | Lysis | | | | | | |
| Fried- | Trans- | 7 | | | | | | |
| rich | fected | | | | | | | |
| | | | 420 | 425 | 451 | 360 | 319 | 412 |
| | | | 478 | 500 | 423 | 376 | 350 | 408 |
| | | | 452 | 505 | 435 | 411 | 419 | 355 |
| | | | 358 | 366 | 438 | 320 | 346 | 336 |
| | | Mean | 426.5 | 449 | 438.75 | 388.75 | 381 | 390.75 |
| | | SD | 52.5468207 | 50.3375208 | 11.5 | 37.7480684 | 42.2453153 | 71.8348801 |
| | | Specif. | 0.1172928 | 0.16891311 | 0.14080672 | −0.01978778 | −0.03297984 | 0.055922 |
| | | Lysis | | | | | | |

TABLE 20
Summary of the measured cytotoxic T-lymphocyte activities

| horse | orf | immunization | 1.booster | 2.booster | 3.booter |
|---|---|---|---|---|---|
| Frieda | 2 | + | + | − | − |
| | 5 | − | + | − | ++ |
| | 7 | + | − | − | − |
| Daggy | 2 | ++ | − | − | +++ |
| | 5 | ++ | + | − | − |
| | 7 | ++ | − | − | − |
| Nelke | 2 | − | − | − | − |
| | 5 | − | − | − | − |
| | 7 | − | − | − | ++ |
| Friedrich | 2 | − | − | − | ++ |
| | 5 | − | − | − | − |
| | 7 | − | − | − | − |
| Jessy | 2 | − | − | − | − |
| | 5 | − | − | +++ | ++ |
| | 7 | + | ++ | − | − |

$x = \frac{{x}_{1} ( 3 : 1 ) + {x}_{2} ( 25 : 1 ) + {x}_{3} ( 50 : 1 )}{3} = after immunization$
$y = \frac{{y}_{1} ( 3 : 1 ) + {x}_{2} ( 25 : 1 ) + {x}_{3} ( 50 : 1 )}{3} = before immunization$
x − y = absolute increase of the specific lysis in percent
− = 0-5%
+ = >5-10%
++ = >10-20%
+++ = °20-30%

TABLE 19
Summary of the measured cytotoxic T-lymphocyte activities

| horse | orf | immunization | 1.booster | 2.booster | 3.booter |
|---|---|---|---|---|---|
| Frieda | 2 | ++ | ++ | − | + |
| | 5 | + | ++ | − | +++ |
| | 7 | ++ | + | + | − |
| Daggy | 2 | +++ | − | − | ++++ |
| | 5 | +++ | ++ | − | − |
| | 7 | +++ | − | − | − |
| Nelke | 2 | + | − | − | − |
| | 5 | + | + | − | − |
| | 7 | − | − | − | +++ |
| Friedrich | 2 | − | − | + | +++ |
| | 5 | + | + | + | − |
| | 7 | + | + | − | − |
| Jessy | 2 | + | + | − | − |
| | 5 | − | − | ++++ | +++ |
| | 7 | ++ | +++ | − | − |

$x = \frac{{x}_{1} ( 3 : 1 ) + {x}_{2} ( 25 : 1 ) + {x}_{3} ( 50 : 1 )}{3} = after immunization$
$y = \frac{{y}_{1} ( 3 : 1 ) + {x}_{2} ( 25 : 1 ) + {x}_{3} ( 50 : 1 )}{3} = before immunization$
x − y = absolute increase of the specific lysis in percent
− = 0%
+ = >0-5%
++ = >5-10%
+++ = °10-20%
++++ = >20-30%

TABLE 18e
2. CTL-Assay, 10./11.08.2000

Prior to Immunization
E:T

| Horse | Cells | ORF | Negative Control | Positive Control | 3 to 1 | 25 to 1 | 50 to 1 |
|---|---|---|---|---|---|---|---|
| Frieda | Transfected | 2 | 121 | 230 | | | |
| | | | 144 | 221 | | | |
| | | | 140 | 222 | | | |
| | | | 153 | 227 | | | |
| | | | 207 | 200 | 110 | 137 | 152 |
| | | | 122 | 212 | 122 | 145 | 137 |
| | | | 121 | 228 | 139 | 168 | 130 |
| | | | 172 | 201 | 139 | 131 | 141 |
| | | Mean. | 194 | 290 | 128.75 | 143 | 140 |
| | | SD | 29.8657109 | 27.1503749 | 10.3077841 | 11.1654228 | 9.20144916 |
| | | Specif. Lysis | | | −0.05 | 0.08571425 | 0.05714283 |
| Frieda | Transfected | 5 | 80 | 118 | | | |
| | | | 87 | 107 | | | |
| | | | 79 | 158 | | | |
| | | | 64 | 134 | | | |
| | | | | 137 | 73 | 81 | 82 |
| | | | 86 | 127 | 64 | 60 | 73 |
| | | | 81 | 132 | 88 | 84 | 92 |
| | | | 96 | 123 | 82 | 104 | |
| | | Mean | 81.2857149 | 126.73 | 84.5 | 86.5 | 85.6665807 |
| | | SD | 6.40966024 | 10.3513282 | 6.3465602 | 0.01285956 | 10.8588581 |
| | | Specif. Lysis | | | 0.07086914 | 0.15683028 | 0.0863503 |
| Freida | Transfected | 7 | | 206 | | | |
| | | | 130 | 307 | | | |
| | | | 130 | 212 | | | |
| | | | 112 | 208 | | | |
| | | | 159 | 210 | 126 | 149 | 140 |
| | | | 149 | 317 | 156 | 194 | 140 |
| | | | | 309 | 144 | 188 | 145 |
| | | | | 264 | 126 | 150 | |
| | | Mean | 131.6 | 281.75 | 138.5 | 146.25 | 144.086037 |
| | | SD | 13.2211051 | 62.5067895 | 14.4598323 | 7.13559164 | 4.60824875 |
| | | Specif. Lysis | | | 0.03088919 | 0.11256248 | 0.10058399 |

| | | | After the 2^{nd} Booster | | | After the 3^{rd} Booster | | |
|---|---|---|---|---|---|---|---|---|
| | | | E:T | | | E:T | | |
| Horse | Cells | ORF | 3 to 1 | 25 to 1 | 50 to 1 | 3 to 1 | 25 to 1 | 50 to 1 |
| Frieda | Trans- | 2 | | | | | | |
| | fected | | | | | | | |
| | | | 130 | 120 | 132 | 131 | 163 | 145 |
| | | | 130 | 165 | 145 | | 149 | 150 |
| | | | 134 | 149 | 143 | 138 | 127 | 127 |
| | | | 141 | 144 | 143 | 144 | 161 | 133 |
| | | Mean. | 133.75 | 140 | 140.75 | 137 | 150 | 138.75 |
| | | SD | 5.10412707 | 8.34523051 | 5.80833259 | 6.65743052 | 16.5327057 | 10.5948101 |
| | | Specif. | −0.60738085 | 0.05714265 | 0.08428571 | 0.02857143 | 0.15238005 | 0.0452381 |
| | | Lysis | | | | | | |
| Frieda | Trans- | 5 | | | | | | |
| | fected | | | | | | | |
| | | | 69 | 72 | 80 | 82 | 105 | 101 |
| | | | | 83 | 80 | 70 | 82 | 90 |
| | | | 82 | 89 | 84 | 93 | 101 | 81 |
| | | | 67 | 83 | 88 | 88 | 85 | |
| | | Mean | 79.3333333 | 80.75 | 87.75 | 84.25 | 86.5 | 80.8685887 |
| | | SD | 81.2386102 | 8.99903283 | 0.08028018 | 7.19559154 | 8.244980 | 10.0180528 |
| | | Specif. | −0.04284918 | −0.01178319 | 0.14218382 | 6.09520031 | 0.57883315 | 0.20033674 |
| | | Lysis | | | | | | |
| Freida | Trans- | 7 | | | | | | |
| | fected | | | | | | | |
| | | | 140 | 133 | 150 | 120 | 103 | 131 |
| | | | 104 | 145 | 147 | 115 | 133 | 148 |
| | | | 145 | 142 | 151 | 110 | 127 | 143 |
| | | | 153 | | 143 | 111 | 151 | 130 |
| | | Mean | 146.5 | 141 | 149.75 | 114 | 170.6 | 135 |
| | | SD | 6.44671155 | 5.58770458 | 0.98861309 | 4.5400057 | 10.8242270 | 0.00007014 |
| | | Specif. | 0.10670885 | 0.07222435 | 0.89049448 | −0.13522850 | −0.02301887 | 0.04017403 |
| | | Lysis | | | | | | |

TABLE 18d
2. CTL-Assay, 10./11.08.2000

Prior to Immunization
E:T

| Horse | Cells | ORF | Negative Control | Positive Control | 3 to 1 | 25 to 1 | 50 to 1 |
|---|---|---|---|---|---|---|---|
| Nelke | Transacted | 2 | 874 | 3272 | 637 | 883 | 644 |
| | | | | 2553 | 676 | 846 | 846 |
| | | | | 2264 | 909 | 715 | 703 |
| | | | | 2928 | 944 | 737 | 689 |
| | | Mean | 874 | 2754.25 | 914.25 | 895.25 | 679.25 |
| | | SD | #DIV:01 | 439.955875 | 71.1354342 | 71.1254342 | 28.5788903 |
| | | Specif. Lysis | | | 0.02140579 | −0.09808785 | −0.10896325 |
| Nelke | Transfected | | 538 | 975 | 608 | 470 | 503 |
| | | | 526 | 821 | 491 | 520 | 455 |
| | | | 540 | 933 | 503 | 478 | 579 |
| | | | 592 | 897 | 825 | 477 | 510 |
| | | Mean | 548.5 | 956.5 | 505.75 | 408.5 | 511.75 |
| | | SD | 28.6135051 | 35.5858385 | 13.2003789 | 22.6342156 | 48.2850343 |
| | | Specif. Lysis | | | −0.10769368 | −0.15479115 | −0.09275184 |
| Nelke | Transfected | | 388 | 1110 | 408 | 502 | 438 |
| | | | 526 | 622 | 514 | 455 | 432 |
| | | | 441 | 892 | 608 | 467 | 499 |
| | | | 376 | 854 | 632 | 513 | 428 |
| | | Mean | 438 | 669.5 | 539.5 | 489.25 | 433 |
| | | SD | 65.4876492 | 118.653899 | 102.389778 | 25.1875828 | 4.454503357 |
| | | Specif. Lysis | | | 0.19403187 | 0.08801258 | −0.00552524 |

| | | | After the 2^{nd} Booster | | | After the 3^{rd} Booster | | |
|---|---|---|---|---|---|---|---|---|
| | | | E:T | | | E:T | | |
| Horse | Cells | ORF | 3 to 1 | 25 to 1 | 50 to 1 | 3 to 1 | 25 to 1 | 50 to 1 |
| Nelke | Trans- | 2 | 825 | 826 | 738 | 480 | 628 | 504 |
| | fected | | | | | | | |
| | | | 773 | 919 | 746 | 552 | 565 | 711 |
| | | | 905 | 836 | 683 | 504 | 611 | 686 |
| | | | 758 | 858 | 603 | 598 | 690 | 630 |
| | | Mean | 614.75 | 607.75 | 710.75 | 558 | 608.5 | 822.75 |
| | | SD | 65.4845883 | 96.5776434 | 28.3240101 | 56.8037557 | 27.2090871 | 31.1839288 |
| | | Specif. | −0.00332403 | −0.332403 | −0.08255801 | −0.15608276 | −0.14852307 | −0.13382585 |
| | | Lysis | | | | | | |
| Nelke | Trans- | | 478 | 505 | 531 | 392 | 450 | 473 |
| | fected | | | | | | | |
| | | | 485 | 520 | 469 | 429 | 554 | 551 |
| | | | 503 | 490 | 489 | 432 | 475 | 503 |
| | | | 484 | 498 | 509 | 458 | 483 | 445 |
| | | Mean | 492 | 901 | 493 | 428 | 492.75 | 492.5 |
| | | SD | 11.4017543 | 16.5529451 | 32.5371582 | 27.5317998 | 42.0347079 | 46.0543156 |
| | | Specif. | −0.14127764 | −0.11918462 | −0.13882054 | −0.2885258 | −0.13943499 | −0.14004814 |
| | | Lysis | | | | | | |
| Nelke | Trans- | | 369 | 410 | 305 | 523 | 553 | 558 |
| | fected | | | | | | | |
| | | | 323 | 482 | 524 | 538 | 630 | 627 |
| | | | 384 | 355 | 501 | 468 | 544 | 515 |
| | | | 451 | 352 | 540 | 649 | 498 | 569 |
| | | Mean | 376.78 | 399.75 | 507.5 | 551.25 | 551.25 | 587.25 |
| | | SD | 65.4518511 | 85.9718881 | 85.1214910 | 70.583039 | 24.1022128 | 48.1474087 |
| | | Specif. | −0.11145034 | −0.05794752 | 0.13402082 | 0.20243574 | 0.17633706 | 0.24601887 |
| | | Lysis | | | | | | |

### REFERENCES

Balasuriya, U. B. R., Hedges, J. F., Nadler, S. A., McCollum, W. H., Timoney, P. J. and MacLachlan, N. J. (1999) Genetic stability of equine arteritis virus during horizontal and vertical transmission in an outbreak of equine viral arteritis. J. Gen. Virol. 80, 1949-1958.

Balasuriya, U. B. R., MacLachlan N. J., DeVries, A. A. F., Rossitto, P. V., and Rottier, P. J. M. (1995). Identification of a neutralization site in the major envelope glycoprotein (G_{L}) of equine arteritis virus. Virology 207, 518-527.
Balasuriya, U. B., Patton, J. F., Rossitto, P. V., Timoney, P. J., McCollum, W. H., and MacLachlan, N. J. (1997). Neutralization determinants of laboratory strains and field isolates of equine arteritis virus: identification of four neutralization sites in the amino-terminal ectodomain of the G(L) envelope glycoprotein. Virology 232, 114-128.
Barry M. A. and Johnston S. A (1997). Biological features of genetic immunization. Vaccine 15, 788-795.
Bradford, M. (1976). A rapid and sensitive method for the quantitation of microgram quantities of proteins utilizing the principle of protein dye binding. Anal. Biochem. 72, 248-254.
den Boon, J. A., Snijder, E. J., Chirnside, E. D., de Vries, A. A., Horzinek, M. C., and Spaan, W. J. (1991). Equine arteritis virus is not a togavirus but belongs to the coronaviruslike superfamily. J Virol 65, 2910-2920.
Gli\check{s}in, V., Crkvenjakov, R., and Byus, C. (1974). Ribonucleic acid isolated by cesium chloride centrifugation. Biochemistry 13, 2633-2637.
Hedges, J. F., Balasuriya, U. B. R., Timoney, P. J., McCollum, W. H., and McLachlan, N. J. (1999). Genetic divergence with emergence of novel phenotypic variants of equine arteritis virus during persistent infection of stallions. J Virol 73, 3672-3681.
Pasquini, S., Xiang, Z., Wang, Y., He, Z., Deng, H., Blaszczyk-Thurin, M., and Ertl, H. C. (1997) Cytokines and costimulatory molecules as genetic adjuvants. Immunol. Cell Biol. 75, 397-401.
Patton, J. F., Balasuriya, U. B. R., Hedges, J. F., Schweidler, T. M., Hullinger, P. J., and MacLachlan, N. J. (1999). Phylogentic characterization of a highly attenuated strain of equine ateritis virus from the semen of a persistently infected standardbred stallion. Arch Virol 144, 817-827.
Pedersen, K. W., Van Der Meer, Y. Roos, N., and Snijder, E. (1999). Open reding frame 1a-encoded subunit of the arterivirus replicase induce endoplasmic reticulum-derived double-membrane vesicles which carry the viral replication complex. J Virol 73, 2016-2026.
Pirzadeh, B., and Dea, S. (1998). Immune response in pigs vaccinated with plasmid DNA encoding ORF5 of porcine reproductive and respiratory syndrome virus. J. Gen. Virol. 79, 989-999.
Pycock, J. F. (1998). Equine viral arteritis risk from imported semen. The Veterinary Record, 26, 699.
Rösen-Wolff, A., Ben-Hur, T., Becker, Y., and Darai, G. (1988). Comparative analysis of the transcripts mapped in the BamHI DNA fragment B of avirulent HSV-1 HFEM, virulent HSV-1 F, and their intratypic recombinant viruses. Virus Res. 10, 315-324.
Rösen-Wolff, A., Ben-Hur, T., Becker, Y., and Darai, G. (1988) Comparative analysis of the transcripts mapped in the BamHI DNA fragment B of avirulent HSV-1 HFEM, virulent HSV-1 F, and their intratypic recombinant viruses. Virus Res. 10, 315-324.
Rösen-Wolff, A., Scholz, J. and Darai, G. (1989) Organotropism of latent herpes simplex virus type 1 is correlated to the presence of a 1.5 kb RNA transcript mapped within the BamHI DNA fragment B (0.738 to 0.809 map units). Virus Res. 12, 43-52.
Rösen-Wolff, A. and Darai, G. (1991) Identification and mapping of the UL56 gene transcript of herpes simplex virus type 1. Virus Res. 19, 115-126.
Stadejek, T., Björklund, H., Ros Bascunana, C., Ciabatti, I. M., Scicluna, M. T., Amaddeo, D., McCollum, W. H., Autorino, G. L., Timoney, P. J. Paton, D. J., Klingeborn, B., and Belak, S. (1999). Genetic diversity of equine ateritis virus. J. Gen. Virol. 80, 691-699.
Snijder, E., Van Tol, H., Pedersen, K. W., Raamsman, M. J. B., and De Vries, A. A. F. (1999). Idenification of a novel structural protein of arteriviruses. J Virol 73, 6335-6345.
Thompson, J. D., Gibson, T. J., Plewniak, F., Jeanmougin, F., and Higgins, D. G. (1997). The CLUSTAL_X windows interface: flexible strategies for multiple sequence alignment aided by quality analysis tools. Nucleic Acids Res. 25, 4876-4882.
van der Meer, Y., Van Tol, H. Locker J. K., and Snijder, E. (1998). DORF1a-encoded replicase subunits are involved in the membrane association of the arterivirus replication complex. Journal of Virology 72, 6689-6698.
van Dinten, L. C., Rensen, S., Gorbalenya, A. E. and Snijder, E. J. (1999). Proteolytic processing of the open reading frame 1b-encoded part of arterivirus replicase is mediated by nsp4 Serine protease and is essential for virus replication. J Virol 73, 2027-2037.
Will, H., Cattaneo, R., Koch, H. G., Darai, G., Schaller, H., Schellekens H., van Eerd, P. M. C. A., and Deinhardt, F. (1982). Cloned HBV DNA causes hepatitis in a chimpanzee Nature 299, 740-742.
Will, H., Cattaneo, R., Darai, G., Deinhardt, F., Schellekens H., and Schaller, H. (1985). Infectious hepatitis B virus from cloned DNA of known nucleotide sequence, Proc. Natn. Acad. Sci. U.S.A. 82, 891-895.

1. A vaccine composition which is protective against equine arterivirus (EAV) infections in horses and induces a cellular immune response, comprising a open reading frame nucleic acid (ORF) 2, ORF 5 and/or ORF7 of EAV.

2. The vaccine composition according to claim 1, wherein said vaccine composition comprises ORF 2, ORF 5 and ORF7 of EAV.

3. The vaccine composition according to claim 1, wherein said vaccine composition further comprises one or several ORF(s) selected from the group of ORF 1a, ORF 1b, ORF 3, ORF 4, ORF 6.

4. The vaccine composition according to claim 1, wherein said nucleic acid is cDNA.

5. The vaccine composition according to claim 1, wherein said vaccine composition comprises one or several nucleic acid vectors each comprising said ORF or ORFs.

6. The vaccine composition according to claim 5, wherein said vector(s) is/are expression vector(s).

7. The vaccine composition according to claim 6, wherein said expression vector(s) comprise(s) a eukaryotic cis-acting transcription/translation sequence functionally linked to said ORF(s).

8. The vaccine composition according to claim 7, wherein said expression vector is selected from the group of pCR3.1, pcDNA3. I/His A, pcDNA3.1/His B, pcDNA3.1/His C, and pDisplay (pD).

9. The vaccine composition according to claim 1, further comprising the nucleic acid encoding equine interleukin 2 (IL-2) or a vector or expression vector comprising said nucleic acid encoding IL-2.

10. The vaccine composition according to claim 1, further comprising pharmaceutically acceptable carrier or excipient.

11. The vaccine composition according to claim 1, further comprising one or several adjuvants selected from the group of Muramyl Dipeptide (MDP), Montanide 720, Poly Inosine:Cytosine (Poly I:C) or plasmid DNA comprising unmethylated cytosine, guanine dinucleotide sequence motifs (CpG).

12. The vaccine composition according to claim 1, consisting of expression vectors comprising ORF2, ORF5 and ORF7 of EAV, respectively, and optionally carrier, excipients or adjuvants and an expression vector comprising the nucleic acid encoding IL-2.

13. The vaccine composition according to claim 1, wherein ORF 2 is SEQ ID No. 2, ORF 5 is SEQ ID No. 5 or SEQ ID No. 9 and ORF 7 is SEQ ID No. 7.

14. The vaccine composition according to claim 1, wherein the nucleic acid or nucleic acid vector or expression vector is encapsulated into cationic liposomes.

15. A nucleic acid vector comprising nucleic acid selected from the group of ORF 1a, ORF 1b, ORF 2, ORF 3, ORF 4, ORF 5, ORF 6 and/or ORF7 of EAV.

16. The nucleic acid vector according to claim 15, wherein said nucleic acid is DNA.

17. The nucleic acid vector according to claim 15, wherein said nucleic acid vector is an expression vector.

18. The nucleic acid vector according to claim 17, wherein said expression vector comprises a eukaryotic cis-acting transcription/translation sequence functionally linked to said nucleic acid(s) specific for said ORF(s).

19. The nucleic acid vector according to claim 17, wherein said expression vector is selected from the group of pCR3.1, pcDNA3.1/His A, pcDNA3.1/His B, pcDNA3.1/His C, and pDisplay (pD).

20. The nucleic acid vector according to claim 15, wherein said nucleic acid vector comprises a nucleic acid selected from the group of SEQ ID No. 2, SEQ ID No. 5, SEQ ID No. 9 and/or SEQ ID No. 7.

21. A method for prophylaxis or treatment of EAV infection in a horse, comprising
(i) coating one or several nucleic acid vector(s) according to any one of claims 15 to 20 onto carrier particles;
(ii) accelerating the coated carrier particles into epidermal cells of the horse in vivo; and
(iii) inducing a protective or therapeutic immune response in said horse upon or after exposure to EAV; and
(iv) monitoring the reduction of EAV-associated symptoms or the reduction of horizontal or vertical transmission.

22. The method according to claim 21, wherein the carrier particles are gold.

23. A method for prophylaxis or treatment of EAV infection in a horse, comprising
(i) injecting a vaccine composition according to claim 1 or one or several nucleic acid vector(s) according to any one of claim 15 into muscular cells of the horse in vivo; and
(ii) inducing a protective or therapeutic immune response in said horse upon or after exposure to EAV, and
(iii) monitoring the reduction of EAV-associated symptoms or the reduction of horizontal or vertical transmission.


SEQUENCE LISTING (61 sequences)

SEQ ID NO: 1 - DNA, 9031 bp, Artificial Sequence
  ORF1a and 1b of EAV
taaaacttttgccacctgaccgccgtgaggctggtttgcggttgtattacaaccattaccgcgaacaaaggaccgggtggctgtctaaaacaggacttcgcttatggcttggagacctgggtttgggcatcaatgcgagctctggagggctgaaattccacattatgaggggttcgcctcagcgagcttggcatatcacaacacgcagctgcaagctgaagagctactacgtttgtgacatctctgaagcagactggtcctgtttgcctgctggcaactacggcggctacaatccaccaggggacggagcttgcggttacaggtgcttggccttcatgaatggcgccactgttgtgtcggctggttgcagttctgacttgtggtgtgatgatgagttggcttatcgagtctttcaattgtcacccacgttcacggttaccatcccaggtgggcgagtttgtccgaatgccaagtacgcaatgatttgtgacaagcagcactggcgcgtcaaacgtgcaaagggcgtcggcctgtgtctcgatgaaagctgtttcaggggcatctgcaattgccaacgcatgagtggaccaccacctgcacccgtgtcagccgccgtgttagatcacatactggaggcggcgacgtttggcaacgttcgcgtggttacacctgaagggcagccacgccccgtaccagcgccgcgagttcgtcccagcgccaactcttctggagatgtcaaagatccggcgcccgttccgccagtaccaaaaccaaggaccaagcttgccacaccgaacccaactcaggcgcccatcccagcaccgcgcacgcgacttcaaggggcctcaacacaggagccactggcgagtgcaggagttgcttctgactcggcacctaaatggcgtgtggccaaaactgtgtacagctccgcggagcgctttcggaccgaactggtacaacgtgctcggtccgttggggacgttcttgttcaagcgctaccgctcaaaaccccagcagtgcagcggtataccatgactctgaagatgatgcgttcacgcttcagttggcactgcgacgtgtggtaccctttggctgtaatcgcttgtttgctccctatatggccatctcttgctttgctccttagctttgccattgggttgatacccagtgtgggcaataatgttgttctgacagcgcttctggtttcatcagctaattatgttgcgtcaatggaccatcaatgtgaaggtgcggcttgcttagccttgctggaagaagaacactattatagagcggtccgttggcgcccgattacaggcgcgctgtcgcttgtgctcaatttactggggcaggtaggctatgtagctcgttccacctttgatgcagcttatgttccttgcactgtgttcgatctttgcagctttgctattctgtacctctgccgcaatcgttgctggagatgcttcggacgctgtgtgcgagttgggcctgccacgcatgttttgggctccaccgggcaacgagtttccaaactggcgctcattgatttgtgtgaccacttttcaaagcccaccatcgatgttgtgggcatggcaactggttggagcggatgttacacaggaaccgccgcaatggagcgtcagtgtgcctctacggtggaccctcactcgttcgaccagaagaaggcaggagcgactgtttacctcaccccccctgtcaacagcgggtcagcgctgcagtgcctcaatgtcatgtggaagcgaccaattgggtccactgtccttggggaacaaacaggagctgttgtgacggcggtcaagagtatctctttctcacctccctgctgcgtctctaccactttgcccacccgacccggtgtgaccgttgtcgaccatgctctttacaaccggttgactgcttcaggggtcgatcccgctttattgcgtgttgggcaaggtgattttctaaaacttaatccggggttccggctgataggtggatggatttatgggatatgctattttgtgttggtggttgtgtcaacttttacctgcttacctatcaaatgtggcattggcacccgcgaccctttctgccgcagagtgttttctgtacccgtcaccaagacccaagagcactgccatgctggaatgtgtgctagcgctgaaggcatctctctggactctctggggttaactcagttacaaagttactggatcgcagccgtcactagcggattagtgatcttgttggtctgccaccgcctggccatcagcgccttggacttgttgactctagcttcccctttagtgttgcttgtgttcccttgggcatctgtggggcttttacttgcttgcagtctcgctggtgctgctgtgaaaatacagttgttggcgacgctttttgtgaatctgttctttccccaagctacccttgtcactatgggatactgggcgtgcgtggcggctttggccgtttacagtttgatgggcttgcgagtgaaagtgaatgtgcccatgtgtgtgacacctgcccattttctgctgctggcgaggtcagctggacagtcaagagagcagatgctccgggtcagcgctgctgcccccaccaattcactgcttggagtggctcgtgattgttatgtcacaggcacaactcggctgtacatacccaaggaaggcgggatggtgtttgaagggctattcaggtcaccgaaggcgcgcggcaacgtcggcttcgtggctggtagcagctacggcacagggtcagtgtggaccaggaacaacgaggtcgtcgtactgacagcgtcacacgtggttggccgcgctaacatggccactctgaagatcggtgacgcaatgctgactctgactttcaaaaagaatggcgacttcgccgaggcagtgacgacacagtccgagctcccaggcaattggccacagttgcatttcgcccaaccaacaaccgggcccgcttcatggtgcactgccacaggagatgaagaaggcttgctcagtggcgaggtttgtctggcgtggactactagtggcgactctggatctgcagtggttcagggtgacgctgtggtaggggtccacaccggttcgaacacaagtggtgttgcctacgtgaccaccccaagcggaaaactccttggcgccgacaccgtgactttgtcatcactgtcaaagcatttcacaggccctttgacatcaatcccgaaggacatccctgacaacattattgccgatgttgatgctgttcctcgttctctggccatgctgattgatggcttatccaatagagagagcagcctttctggacctcagttgttgttaattgcttgttttatgtggtcttatcttaaccaacctgcttacttgccttatgtgctgggcttctttgccgctaacttcttcctgccaaaaagtgttggccgccctgtggtcactgggcttctatggttgtgctgcctcttcacaccgctttccatgcgcttgtgcttgttccatctggtctgtgctaccgtcacgggaaacgtgatatctttgtggttctacatcactgccgctggcacgtcttacctttctgagatgtggttcggaggctatcccaccatgttgtttgtgccacggttcctagtgtaccagttccccggctgggctattggcacagtactagcggtatgcagcatcaccatgctggctgctgccctcggtcacaccctgttactggatgtgttctccgcctcaggtcgctttgacaggactttcatgatgaaatacttcctggagggaggagtgaaagagagtgtcaccgcctcagtcacccgcgcttatggcaaaccaattacccaggagagtctcactgcaacattagctgccctcactgatgatgacttccaattcctctctgatgtgcttgactgtcgggccgtccgatcggcaatgaatctgcgtgccgctctcacaagttttcaagtggcgcagtatcgtaacatccttaatgcatccttgcaagtcgatcgtgacgctgctcgtagtcgcagactaatggcaaaactggctgattttgcggttgaacaagaagtaacagctggagaccgtgttgtggttatcgacggtctggaccgcatggctcacttcaaagacgatttggtgctggttcctttgaccaccaaagtagtaggcggttctaggtgcaccatttgtgacgtcgttaaggaagaagccaatgacaccccagttaagccaatgcccagcaggagacgccgcaagggcctgcctaaaggtgctcagttggagtgggaccgtcaccaggaagagaagaggaacgccggtgatgatgattttgcggtctcgaatgattatgtcaagagagtgccaaagtactgggatcccagcgacacccgaggcacgacagtgaaaatcgccggcactacctatcagaaagtggttgactattcaggcaatgtgcattacgtggagcatcaggaagatctgctagactacgtgctgggcaaggggagctatgaaggcctagatcaggacaaagtgttggacctcacaaacatgcttaaagtggaccccacggagctctcctccaaagacaaagccaaggcgcgtcagcttgctcatctgctgttggatctggctaacccagttgaggcagtgaatcagttaaactgagagcgccccacatctttcccggcgatgtggggcgtcggacctttgctgactctaaagacaagggtttcgtggctctacacagtcgcacaatgtttttagctgcccgggactttttatttaacatcaaatttgtgtgcgacgaagagttcacaaagaccccaaaagacacactgcttgggtacgtacgcgcctgccctggttactggtttattttccgtcgtacgcaccggtcgctgattgatgcatactgggacagtatggagtgcgtttacgcgcttcccaccatatctgattttgatgtgagcccaggtgacgtcgcagtgacgggcgagcgatgggattttgaatctcccggaggaggccgtgcaaaacgtctcacagctgatctggtgcacgcttttcaagggttccacggagcctcttattcctatgatgacaaggtggcagctgctgtcagtggtgacccgtatcggtcggacggcgtcttgtataacacccgttggggcaacattccatattctgtcccaaccaatgctttggaagccacagcttgctaccgtgctggatgtgaggccgttaccgacgggaccaacgtcatcgcaacaattgggcccttcccggagcaacaacccataccggacatcccaaagagcgtgcttgacaactgcgctgacatcagctgtgacgctttcatagcgcccgctgcagagacagccctgtgtggagatttagagaaatacaacctatccacgcagggttttgtgttgcctagtgttttctccatggtgcgggcgtacttaaaagaggagattggagacgctccaccactctacttgccatctactgtaccatctaaaaattcacaagccggaattaacggcgctgagtttcctacaaagtctttacagagctactgtttgattgatgacatggtgtcacagtccatgaaaagcaatctacaaaccgccaccatggcgacttgtaaacggcaatactgttccaaatacaagattaggagcattctgggcaccaacaattacattggcctaggtttgcgtgcctgcctttcgggggttacggccgcattccaaaaagctggaaaggatgggtcaccgatttatttgggcaagtcaaaattcgacccgataccagctcctgacaagtactgccttgaaacagacctggagagttgtgatcgctccaccccggctttggtgcgttggttcgctactaatcttatttttgagctagctggccagcccgagttggtgcacagctacgtgttgaattgctgtcacgatctagttgtggcgggtagtgtagcattcaccaaacgcgggggtttgtcatctggagaccctatcacttccatttccaataccatctattcattggtgctgtacacccagcacatgttgctatgtggacttgaaggctatttcccagagattgcagaaaaatatcttgatggcagcctggagctgcgggacatgttcaagtacgttcgagtgtacatctactcggacgatgtggttctaaccacacccaaccagcattacgcggccagctttgaccgctgggtcccccacctgcaggcgctgctaggtttcaaggttgacccaaagaaaactgtgaacaccagctccccttcctttttgggctgccggttcaagcaagtggacggcaagtgttatctagccagtcttcaggaccgcgttacacgctctctgttataccacattggtgcaaagaatccctcagagtactatgaagctgctgtttccatctttaaggactccattatctgctgtgatgaagactggtggacggacctccatcgacgtatcagtggcgctgcgcgtaccgacggagttgagttccccaccattgaaatgttaacatccttccgcaccaagcagtatgagagtgccgtgtgcacagtttgtggggccgcccccgtggccaagtctgcttgtggagggtggttctgtggcaattgtgtcccgtaccacgcgggtcattgtcacacaacctcgctcttcgccaactgcgggcacgacatcatgtaccgctccacttactgcacaatgtgtgagggttccccaaaacagatggtaccaaaagtgcctcacccgatcctggatcatttgctgtgccacattgattacggcagtaaagaggaactaactctggtagtggcggatggtcgaacaacatcaccgcccgggcgctacaaagtgggtcacaaggtagtcgccgtggttgcagatgtgggaggcaacattgtgtttgggtgcggtcctggatcacacatcgcagtaccacttcaggatacgctcaagggcgtggtggtgaataaagctctgaagaacgccgccgcctctgagtacgtggaaggaccccctgggagtgggaagacttttcacctggtcaaagatgtgctagccgtggtcggtagcgcgaccttggttgtgcccacccacgcgtccatgctggactgcatcaacaagctcaaacaagcgggcgccgatccatactttgtggtgcccaagtatacagttcttgactttccccggcctggcagtggaaacatcacagtgcgactgccacaggtcggaaccagtgagggagaaacctttgtggatgaggtggcctacttctcaccagtggatctggcgcgcattttaacccagggtcgagtcaagggttacggtgatttaaatcagctcgggtgcgtcggacccgcgagcgtgccacgtaacctttggctccgacattttgtcagcctggagcccttgcgagtgtgccatcgattcggcgctgctgtgtgtgatttgatcaagggcatttatccttattatgagccagctccacataccactaaagtggtgtttgtgccaaatccagactttgagaaaggtgtagtcatcaccgcctaccacaaagatcgcggtcttggtcaccgcacaattgattcaattcaaggctgtacattccctgttgtgactcttcgactgcccacaccccaatcactgacgcgcccgcgcgcagttgtggcggttactagggcgtctcaggaattatacatctacgacccctttgatcagcttagcgggttgttgaagttcaccaaggaagcagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatctgttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtga

SEQ ID NO: 2 - DNA, 684 bp, Artificial Sequence
  ORF2 of EAV
tgctatttctgtgattgatgcagcgcttttctttctcatgctacttgcattggctgttgttactgtgtttcttttctggctcattgttgccatcggccgcagcttggtggcgcggtgttcacgaggtgcgcgttacagacctgtttaaggatttgcagtgcgacaacctgcgcgcgaaagatgccttcccgagtctgggatatgctctgtcgattggccagtcgaggctatcgtatatgctgcaggattggttgcttgctgcgcaccgcaaggaagttatgccttccaatatcatgcctatgcccggtcttactcctgattgctttgaccatctggagtcttctagctatgctccatttatcaatgcctatcggcaggcaattttgagtcaatacccacaagagctccagctcgaagccatcaactgtaaattgcttgctgtggttgcaccggcattgtatcataattaccatctagccaatttgaccggaccggccacatgggtcgtgcctacagtgggccagttgcactattatgcttcttcctctatttttgcttcatctgtggaagtgttggcagcaataatactactatttgcatgcataccactagtgacacgagtgtacatctcttttacgcggctaatgtcaccttcccgtcgcacttccagcggcactttgccgc

SEQ ID NO: 3 - DNA, 501 bp, Artificial Sequence
  ORF3 of EAV
accggccacatgggtcgtgcctacagtgggccagttgcactattatgcttcttcctctatttttgcttcatctgtggaagtgttggcagcaataatactactatttgcatgcataccactagtgacacgagtgtacatctcttttacgcggctaatgtcaccttcccgtcgcacttccagcggcactttgccgcggcgcaagattttgtagtgcacacgggttatgaatatgccggggtcactatgttagtgcacttgtttgccaacttggttctgacatttccgagcttagttaattgttcccgccctgtgaatgtctttgctaatgcttcttgcgtgcaagtggtttgtagtcataccaactcaactactggcttgggtcaactttctttttcctttgtagatgaagatctacggctgcatatcaggcctactcttatttgttggtttgccttgttgttggtgcactttctacccatgccacgctgcagaggctcgtaa

SEQ ID NO: 4 - DNA, 458 bp, Artificial Sequence
  ORF4 of EAV
tgaagatctacggctgcatatcaggcctactcttatttgttggtttgccttgttgttggtgcactttctacccatgccacgctgcagaggctcgtaattttacttacattagtcatggattgggccacgtgcacggtcatgaggggtgtaggaattttattaatgtcactcattctgcatttctttatcttaatcccaccactcccactgcgccggctataactcattgtttacttctggttctggcagccaaaatggaacacccaaacgctactatctggctgcagctgcagccgtttgggtatcatgtggctggcgatgtcattgtcaacttggaagaggacaagaggcatccttactttaaacttttgagagcgccggctttaccgcttggttttgtggctatagtttatgttcttttacgactggtacgttgggctcaacgatgttatctatga

SEQ ID NO: 5 - DNA, 767 bp, Artificial Sequence
  ORF5 of EAV
tacgttgggctcaacgatgttatctatgattgtattgctattcttgctttggggtgcgccatcacatgcttacttctcatactacaccgctcagcgcttcacagacttcaccttgtgtatgctgacggatcgcggcgttattgccaatttgctgcgatatgatgagcacactgctttgtacaattgttccgccagtaaaacctgttggtattgcacattcctggacgaacagattatcacgtttggaaccgattgtgatgacacctacgcggtcccagttgctgaggtcctggaacaggcgcatggaccgtacagtgcgctgtttgatgacatgcccccttttatttactatggccgtgaattcggcatagttgtgttggatgtgtttatgttctatcccgttttagttctgtttttcttatcagtactaccctatgctacgcttattcttgaaatgtgtgtatctattctgtttataatctatggcatttacagcggggcctacttggccatgggcatatttgcggccacgcttgctatacattcaattgtggtcctccgccaattactgtggttatgcctggcttggcgataccgctgtacgcttcacgcgtcctttatatcagctgaggggaaagtgtaccccgtagaccccggactcccggttgccgccgtgggcaatcggttgttagtcccaggtaggcccactatcgattatgcagtggcctacggcagcaaagtcaaccttgtgaggttgggggcagctg

SEQ ID NO: 6 - DNA, 489 bp, Artificial Sequence
  ORF6 of EAV
atgggagccatagattcattttgtggtgacgggattttaggtgagtatctagattactttattctgtccgtcccactcttgctgttgcttactaggtatgtagcatctgggttagtgtatgttttgactgccttgttctattcctttgtattagcagcttatatttggtttgttatagttggaagagccttttctactgcttatgcttttgtgcttttggctgcttttctgttattagtaatgaggatgattgtgggtatgatgcctcgtcttcggtccattttcaaccatcgccaactggtggtagctgattttgtggacacacctagtggacctgttcccatcccccgctcaactactcaggtagtggttcgcggcaacgggtacaccgcagttggtaacaagcttgtcgatggcgtcaagacgatcacgtccgcaggccgcctcttttcgaaacggacggcggcgacagcctacaagctacaatga

SEQ ID NO: 7 - DNA, 333 bp, Artificial Sequence
  ORF7 of EAV
tggtaacaagcttgtcgatggcgtcaagacgatcacgtccgcaggccgcctcttttcgaaacggacggcggcgacagcctacaagctacaatgacctactgcgcatgtttggtcagatgcgggtccgcaaaccgcccgcgcaacccactcaggctattattgcagagcctggagaccttaggcatgatttaaatcaacaggagcgcgccaccctttcgtcgaacgtacaacggttcttcatgattgggcatggttcactcactgcagatgccggaggactcacgtacaccgtcagttgggttcctaccaaacaaatccagcgcaaagttgcgc

SEQ ID NO: 8 - DNA, 495 bp, Artificial Sequence
  Partial ORF1 sequence
tggcaaccttctccgctactggatttggagggagttttgttagggactggtccctggacttacccgacgcttgtgagcatggcgcgggattgtgctgcgaagtggacggctccaccttatgcgccgagtgttttcgcggttgcgaaggaatggagcaatgtcctggcttgttcatgggactgttaaaactggcttcgccagttccagtgggacataagttcctgattggttggtatcgagctgccaaagtcaccgggcgttacaatttccttgagctgttgcaacaccctgctttcgcccagctgcgtgtggttgatgctaggttagccattgaagaggcaagtgtgtttatttccactgaccacgcgtctgctaagcgtttccctggcgctagatttgcgctgacaccggtgtatgctaacgcttgggttgtgagcccggctgctaacagtttgatagtgaccactgaccaggaacaagatgggttctgctggt

SEQ ID NO: 9 - DNA, 363 bp, Artificial Sequence
  Truncated ORF5 of EAV
tacgttgggctcaacgatgttatctatgattgtattgctattcttgctttggggtgcgccatcacatgcttacttctcatactacaccgctcagcgcttcacagacttcaccttgtgtatgctgacggatcgcggcgttattgccaatttgctgcgatatgatgagcacactgctttgtacaattgttccgccagtaaaacctgttggtattgcacattcctggacgaacagattatcacgtttggaaccgattgtgatgacacctacgcggtcccagttgctgaggtcctggaacaggcgcatggaccgtacagtgcgctgtttgatgacatgcccccttttatttactatggccgtgaatt

SEQ ID NO: 10 - DNA, 12704 bp, Equine arteritis virus
gctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgccatcgtcgtcgatctctatcaactacccttgcgactatggcaaccttctccgctactggatttggagggagttttgttagggactggtccctggacttacccgacgcttgtgagcatggcgcgggattgtgctgcgaagtggacggctccaccttatgcgccgagtgttttcgcggttgcgaaggaatggagcaatgtcctggcttgttcatgggactgttaaaactggcttcgccagttccagtgggacataagttcctgattggttggtatcgagctgccaaagtcaccgggcgttacaatttccttgagctgttgcaacaccctgctttcgcccagctgcgtgtggttgatgctaggttagccattgaagaggcaagtgtgtttatttccactgaccacgcgtctgctaagcgtttccctggcgctagatttgcgctgacaccggtgtatgctaacgcttgggttgtgagcccggctgctaacagtttgatagtgaccactgaccaggaacaagatgggttctgctggttaaaacttttgccacctgaccgccgtgaggctggtttgcggttgtattacaaccattaccgcgaacaaaggaccgggtggctgtctaaaacaggacttcgcttatggcttggagacctgggtttgggcatcaatgcgagctctggagggctgaaattccacattatgaggggttcgcctcagcgagcttggcatatcacaacacgcagctgcaagctgaagagctactacgtttgtgacatctctgaagcagactggtcctgtttgcctgctggcaactacggcggctacaatccaccaggggacggagcttgcggttacaggtgcttggccttcatgaatggcgccactgttgtgtcggctggttgcagttctgacttgtggtgtgatgatgagttggcttatcgagtctttcaattgtcacccacgttcacggttaccatcccaggtgggcgagtttgtccgaatgccaagtacgcaatgatttgtgacaagcagcactggcgcgtcaaacgtgcaaagggcgtcggcctgtgtctcgatgaaagctgtttcaggggcatctgcaattgccaacgcatgagtggaccaccacctgcacccgtgtcagccgccgtgttagatcacatactggaggcggcgacgtttggcaacgttcgcgtggttacacctgaagggcagccacgccccgtaccagcgccgcgagttcgtcccagcgccaactcttctggagatgtcaaagatccggcgcccgttccgccagtaccaaaaccaaggaccaagcttgccacaccgaacccaactcaggcgcccatcccagcaccgcgcacgcgacttcaaggggcctcaacacaggagccactggcgagtgcaggagttgcttctgactcggcacctaaatggcgtgtggccaaaactgtgtacagctccgcggagcgctttcggaccgaactggtacaacgtgctcggtccgttggggacgttcttgttcaagcgctaccgctcaaaaccccagcagtgcagcggtataccatgactctgaagatgatgcgttcacgcttcagttggcactgcgacgtgtggtaccctttggctgtaatcgcttgtttgctccctatatggccatctcttgctttgctccttagctttgccattgggttgatacccagtgtgggcaataatgttgttctgacagcgcttctggtttcatcagctaattatgttgcgtcaatggaccatcaatgtgaaggtgcggcttgcttagccttgctggaagaagaacactattatagagcggtccgttggcgcccgattacaggcgcgctgtcgcttgtgctcaatttactggggcaggtaggctatgtagctcgttccacctttgatgcagcttatgttccttgcactgtgttcgatctttgcagctttgctattctgtacctctgccgcaatcgttgctggagatgcttcggacgctgtgtgcgagttgggcctgccacgcatgttttgggctccaccgggcaacgagtttccaaactggcgctcattgatttgtgtgaccacttttcaaagcccaccatcgatgttgtgggcatggcaactggttggagcggatgttacacaggaaccgccgcaatggagcgtcagtgtgcctctacggtggaccctcactcgttcgaccagaagaaggcaggagcgactgtttacctcaccccccctgtcaacagcgggtcagcgctgcagtgcctcaatgtcatgtggaagcgaccaattgggtccactgtccttggggaacaaacaggagctgttgtgacggcggtcaagagtatctctttctcacctccctgctgcgtctctaccactttgcccacccgacccggtgtgaccgttgtcgaccatgctctttacaaccggttgactgcttcaggggtcgatcccgctttattgcgtgttgggcaaggtgattttctaaaacttaatccggggttccggctgataggtggatggatttatgggatatgctattttgtgttggtggttgtgtcaacttttacctgcttacctatcaaatgtggcattggcacccgcgaccctttctgccgcagagtgttttctgtacccgtcaccaagacccaagagcactgccatgctggaatgtgtgctagcgctgaaggcatctctctggactctctggggttaactcagttacaaagttactggatcgcagccgtcactagcggattagtgatcttgttggtctgccaccgcctggccatcagcgccttggacttgttgactctagcttcccctttagtgttgcttgtgttcccttgggcatctgtggggcttttacttgcttgcagtctcgctggtgctgctgtgaaaatacagttgttggcgacgctttttgtgaatctgttctttccccaagctacccttgtcactatgggatactgggcgtgcgtggcggctttggccgtttacagtttgatgggcttgcgagtgaaagtgaatgtgcccatgtgtgtgacacctgcccattttctgctgctggcgaggtcagctggacagtcaagagagcagatgctccgggtcagcgctgctgcccccaccaattcactgcttggagtggctcgtgattgttatgtcacaggcacaactcggctgtacatacccaaggaaggcgggatggtgtttgaagggctattcaggtcaccgaaggcgcgcggcaacgtcggcttcgtggctggtagcagctacggcacagggtcagtgtggaccaggaacaacgaggtcgtcgtactgacagcgtcacacgtggttggccgcgctaacatggccactctgaagatcggtgacgcaatgctgactctgactttcaaaaagaatggcgacttcgccgaggcagtgacgacacagtccgagctcccaggcaattggccacagttgcatttcgcccaaccaacaaccgggcccgcttcatggtgcactgccacaggagatgaagaaggcttgctcagtggcgaggtttgtctggcgtggactactagtggcgactctggatctgcagtggttcagggtgacgctgtggtaggggtccacaccggttcgaacacaagtggtgttgcctacgtgaccaccccaagcggaaaactccttggcgccgacaccgtgactttgtcatcactgtcaaagcatttcacaggccctttgacatcaatcccgaaggacatccctgacaacattattgccgatgttgatgctgttcctcgttctctggccatgctgattgatggcttatccaatagagagagcagcctttctggacctcagttgttgttaattgcttgttttatgtggtcttatcttaaccaacctgcttacttgccttatgtgctgggcttctttgccgctaacttcttcctgccaaaaagtgttggccgccctgtggtcactgggcttctatggttgtgctgcctcttcacaccgctttccatgcgcttgtgcttgttccatctggtctgtgctaccgtcacgggaaacgtgatatctttgtggttctacatcactgccgctggcacgtcttacctttctgagatgtggttcggaggctatcccaccatgttgtttgtgccacggttcctagtgtaccagttccccggctgggctattggcacagtactagcggtatgcagcatcaccatgctggctgctgccctcggtcacaccctgttactggatgtgttctccgcctcaggtcgctttgacaggactttcatgatgaaatacttcctggagggaggagtgaaagagagtgtcaccgcctcagtcacccgcgcttatggcaaaccaattacccaggagagtctcactgcaacattagctgccctcactgatgatgacttccaattcctctctgatgtgcttgactgtcgggccgtccgatcggcaatgaatctgcgtgccgctctcacaagttttcaagtggcgcagtatcgtaacatccttaatgcatccttgcaagtcgatcgtgacgctgctcgtagtcgcagactaatggcaaaactggctgattttgcggttgaacaagaagtaacagctggagaccgtgttgtggttatcgacggtctggaccgcatggctcacttcaaagacgatttggtgctggttcctttgaccaccaaagtagtaggcggttctaggtgcaccatttgtgacgtcgttaaggaagaagccaatgacaccccagttaagccaatgcccagcaggagacgccgcaagggcctgcctaaaggtgctcagttggagtgggaccgtcaccaggaagagaagaggaacgccggtgatgatgattttgcggtctcgaatgattatgtcaagagagtgccaaagtactgggatcccagcgacacccgaggcacgacagtgaaaatcgccggcactacctatcagaaagtggttgactattcaggcaatgtgcattacgtggagcatcaggaagatctgctagactacgtgctgggcaaggggagctatgaaggcctagatcaggacaaagtgttggacctcacaaacatgcttaaagtggaccccacggagctctcctccaaagacaaagccaaggcgcgtcagcttgctcatctgctgttggatctggctaacccagttgaggcagtgaatcagttaaactgagagcgccccacatctttcccggcgatgtggggcgtcggacctttgctgactctaaagacaagggtttcgtggctctacacagtcgcacaatgtttttagctgcccgggactttttatttaacatcaaatttgtgtgcgacgaagagttcacaaagaccccaaaagacacactgcttgggtacgtacgcgcctgccctggttactggtttattttccgtcgtacgcaccggtcgctgattgatgcatactgggacagtatggagtgcgtttacgcgcttcccaccatatctgattttgatgtgagcccaggtgacgtcgcagtgacgggcgagcgatgggattttgaatctcccggaggaggccgtgcaaaacgtctcacagctgatctggtgcacgcttttcaagggttccacggagcctcttattcctatgatgacaaggtggcagctgctgtcagtggtgacccgtatcggtcggacggcgtcttgtataacacccgttggggcaacattccatattctgtcccaaccaatgctttggaagccacagcttgctaccgtgctggatgtgaggccgttaccgacgggaccaacgtcatcgcaacaattgggcccttcccggagcaacaacccataccggacatcccaaagagcgtgcttgacaactgcgctgacatcagctgtgacgctttcatagcgcccgctgcagagacagccctgtgtggagatttagagaaatacaacctatccacgcagggttttgtgttgcctagtgttttctccatggtgcgggcgtacttaaaagaggagattggagacgctccaccactctacttgccatctactgtaccatctaaaaattcacaagccggaattaacggcgctgagtttcctacaaagtctttacagagctactgtttgattgatgacatggtgtcacagtccatgaaaagcaatctacaaaccgccaccatggcgacttgtaaacggcaatactgttccaaatacaagattaggagcattctgggcaccaacaattacattggcctaggtttgcgtgcctgcctttcgggggttacggccgcattccaaaaagctggaaaggatgggtcaccgatttatttgggcaagtcaaaattcgacccgataccagctcctgacaagtactgccttgaaacagacctggagagttgtgatcgctccaccccggctttggtgcgttggttcgctactaatcttatttttgagctagctggccagcccgagttggtgcacagctacgtgttgaattgctgtcacgatctagttgtggcgggtagtgtagcattcaccaaacgcgggggtttgtcatctggagaccctatcacttccatttccaataccatctattcattggtgctgtacacccagcacatgttgctatgtggacttgaaggctatttcccagagattgcagaaaaatatcttgatggcagcctggagctgcgggacatgttcaagtacgttcgagtgtacatctactcggacgatgtggttctaaccacacccaaccagcattacgcggccagctttgaccgctgggtcccccacctgcaggcgctgctaggtttcaaggttgacccaaagaaaactgtgaacaccagctccccttcctttttgggctgccggttcaagcaagtggacggcaagtgttatctagccagtcttcaggaccgcgttacacgctctctgttataccacattggtgcaaagaatccctcagagtactatgaagctgctgtttccatctttaaggactccattatctgctgtgatgaagactggtggacggacctccatcgacgtatcagtggcgctgcgcgtaccgacggagttgagttccccaccattgaaatgttaacatccttccgcaccaagcagtatgagagtgccgtgtgcacagtttgtggggccgcccccgtggccaagtctgcttgtggagggtggttctgtggcaattgtgtcccgtaccacgcgggtcattgtcacacaacctcgctcttcgccaactgcgggcacgacatcatgtaccgctccacttactgcacaatgtgtgagggttccccaaaacagatggtaccaaaagtgcctcacccgatcctggatcatttgctgtgccacattgattacggcagtaaagaggaactaactctggtagtggcggatggtcgaacaacatcaccgcccgggcgctacaaagtgggtcacaaggtagtcgccgtggttgcagatgtgggaggcaacattgtgtttgggtgcggtcctggatcacacatcgcagtaccacttcaggatacgctcaagggcgtggtggtgaataaagctctgaagaacgccgccgcctctgagtacgtggaaggaccccctgggagtgggaagacttttcacctggtcaaagatgtgctagccgtggtcggtagcgcgaccttggttgtgcccacccacgcgtccatgctggactgcatcaacaagctcaaacaagcgggcgccgatccatactttgtggtgcccaagtatacagttcttgactttccccggcctggcagtggaaacatcacagtgcgactgccacaggtcggaaccagtgagggagaaacctttgtggatgaggtggcctacttctcaccagtggatctggcgcgcattttaacccagggtcgagtcaagggttacggtgatttaaatcagctcgggtgcgtcggacccgcgagcgtgccacgtaacctttggctccgacattttgtcagcctggagcccttgcgagtgtgccatcgattcggcgctgctgtgtgtgatttgatcaagggcatttatccttattatgagccagctccacataccactaaagtggtgtttgtgccaaatccagactttgagaaaggtgtagtcatcaccgcctaccacaaagatcgcggtcttggtcaccgcacaattgattcaattcaaggctgtacattccctgttgtgactcttcgactgcccacaccccaatcactgacgcgcccgcgcgcagttgtggcggttactagggcgtctcaggaattatacatctacgacccctttgatcagcttagcgggttgttgaagttcaccaaggaagcagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatctgttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtgatgggcttagtgtggtcactgatttcaaattctattcagactattattgctgattttgctatttctgtgattgatgcagcgcttttctttctcatgctacttgcattggctgttgttactgtgtttcttttctggctcattgttgccatcggccgcagcttggtggcgcggtgttcacgaggtgcgcgttacagacctgtttaaggatttgcagtgcgacaacctgcgcgcgaaagatgccttcccgagtctgggatatgctctgtcgattggccagtcgaggctatcgtatatgctgcaggattggttgcttgctgcgcaccgcaaggaagttatgccttccaatatcatgcctatgcccggtcttactcctgattgctttgaccatctggagtcttctagctatgctccatttatcaatgcctatcggcaggcaattttgagtcaatacccacaagagctccagctcgaagccatcaactgtaaattgcttgctgtggttgcaccggcattgtatcataattaccatctagccaatttgaccggaccggccacatgggtcgtgcctacagtgggccagttgcactattatgcttcttcctctatttttgcttcatctgtggaagtgttggcagcaataatactactatttgcatgcataccactagtgacacgagtgtacatctcttttacgcggctaatgtcaccttcccgtcgcacttccagcggcactttgccgcggcgcaagattttgtagtgcacacgggttatgaatatgccggggtcactatgttagtgcacttgtttgccaacttggttctgacatttccgagcttagttaattgttcccgccctgtgaatgtctttgctaatgcttcttgcgtgcaagtggtttgtagtcataccaactcaactactggcttgggtcaactttctttttcctttgtagatgaagatctacggctgcatatcaggcctactcttatttgttggtttgccttgttgttggtgcactttctacccatgccacgctgcagaggctcgtaattttacttacattagtcatggattgggccacgtgcacggtcatgaggggtgtaggaattttattaatgtcactcattctgcatttctttatcttaatcccaccactcccactgcgccggctataactcattgtttacttctggttctggcagccaaaatggaacacccaaacgctactatctggctgcagctgcagccgtttgggtatcatgtggctggcgatgtcattgtcaacttggaagaggacaagaggcatccttactttaaacttttgagagcgccggctttaccgcttggttttgtggctatagtttatgttcttttacgactggtacgttgggctcaacgatgttatctatgattgtattgctattcttgctttggggtgcgccatcacatgcttacttctcatactacaccgctcagcgcttcacagacttcaccttgtgtatgctgacggatcgcggcgttattgccaatttgctgcgatatgatgagcacactgctttgtacaattgttccgccagtaaaacctgttggtattgcacattcctggacgaacagattatcacgtttggaaccgattgtgatgacacctacgcggtcccagttgctgaggtcctggaacaggcgcatggaccgtacagtgcgctgtttgatgacatgcccccttttatttactatggccgtgaattcggcatagttgtgttggatgtgtttatgttctatcccgttttagttctgtttttcttatcagtactaccctatgctacgcttattcttgaaatgtgtgtatctattctgtttataatctatggcatttacagcggggcctacttggccatgggcatatttgcggccacgcttgctatacattcaattgtggtcctccgccaattactgtggttatgcctggcttggcgataccgctgtacgcttcacgcgtcctttatatcagctgaggggaaagtgtaccccgtagaccccggactcccggttgccgccgtgggcaatcggttgttagtcccaggtaggcccactatcgattatgcagtggcctacggcagcaaagtcaaccttgtgaggttgggggcagctgaggtatgggagccatagattcattttgtggtgacgggattttaggtgagtatctagattactttattctgtccgtcccactcttgctgttgcttactaggtatgtagcatctgggttagtgtatgttttgactgccttgttctattcctttgtattagcagcttatatttggtttgttatagttggaagagccttttctactgcttatgcttttgtgcttttggctgcttttctgttattagtaatgaggatgattgtgggtatgatgcctcgtcttcggtccattttcaaccatcgccaactggtggtagctgattttgtggacacacctagtggacctgttcccatcccccgctcaactactcaggtagtggttcgcggcaacgggtacaccgcagttggtaacaagcttgtcgatggcgtcaagacgatcacgtccgcaggccgcctcttttcgaaacggacggcggcgacagcctacaagctacaatgacctactgcgcatgtttggtcagatgcgggtccgcaaaccgcccgcgcaacccactcaggctattattgcagagcctggagaccttaggcatgatttaaatcaacaggagcgcgccaccctttcgtcgaacgtacaacggttcttcatgattgggcatggttcactcactgcagatgccggaggactcacgtacaccgtcagttgggttcctaccaaacaaatccagcgcaaagttgcgcctccagcagggccgtaagacgtggatattctcctgtgtggcgtcatgttgaagtagttattagccacccaggaacc

SEQ ID NO: 11 - DNA, 33 bp, Artificial Sequence
  Oligonucleotide primer
gggtctagagtcaccacaaaatgaatctatggc

SEQ ID NO: 12 - DNA, 34 bp, Artificial Sequence
  Oligonucleotide primer
ggggggatcccctttgtagatgaagatctacggc

SEQ ID NO: 13 - DNA, 31 bp, Artificial Sequence
  Oligonucleotide primer
gggggatcctggtacgttgggctcaacgatg

SEQ ID NO: 14 - DNA, 43 bp, Artificial Sequence
  Oligonucleotide primer
gggggtctagagcaatacaatcatagataacatcgttgagccc

SEQ ID NO: 15 - DNA, 32 bp, Artificial Sequence
  Oligonucleotide primer
gggtctagaccacacaggagaatatccacgtc

SEQ ID NO: 16 - DNA, 33 bp, Artificial Sequence
  Oligonucleotide primer
ggggggatcccagctgaggtatgggagccatag

SEQ ID NO: 17 - DNA, 32 bp, Artificial Sequence
  Oligonucleotide primer
gggggatcccgcagttggtaacaagcttgtcg

SEQ ID NO: 18 - DNA, 34 bp, Artificial Sequence
  Oligonucleotide primer
ggggggaattccatgcgcagtaggtcattgtagc

SEQ ID NO: 19 - DNA, 33 bp, Artificial Sequence; identical to SEQ ID NO: 11
  Oligonucleotide primer

SEQ ID NO: 20 - DNA, 33 bp, Artificial Sequence
  Oligonucleotide primer
gggggggtcgactggctcccatacctcagctgc

SEQ ID NO: 21 - DNA, 31 bp, Artificial Sequence; identical to SEQ ID NO: 13
  Oligonucleotide primer

SEQ ID NO: 22 - DNA, 36 bp, Artificial Sequence
  Oligonucleotide primer
ggggggagatctatggcgtcaagacgatcacgtccg

SEQ ID NO: 23 - DNA, 31 bp, Artificial Sequence
  Oligonucleotide primer
gggggatccatttctgtgattgatgcagcgc

SEQ ID NO: 24 - DNA, 33 bp, Artificial Sequence
  Oligonucleotide primer
gggggggtcgaccggccctgctggaggcgcaac

SEQ ID NO: 25 - DNA, 36 bp, Artificial Sequence
  Oligonucleotide primer
gggtctagaggcatattcataacccgtgtgcactac

SEQ ID NO: 26 - DNA, 36 bp, Artificial Sequence
  Oligonucleotide primer
ggggggagatctatgcagcgcttttctttctcatgc

SEQ ID NO: 27 - DNA, 34 bp, Artificial Sequence
  Oligonucleotide primer
gggggatcctttgaccggaccggccacatgggtc

SEQ ID NO: 28 - DNA, 35 bp, Artificial Sequence
  Oligonucleotide primer
gggggggtcgaccaaaatcttgcgccgcggcaaag

SEQ ID NO: 29 - DNA, 36 bp, Artificial Sequence
  Oligonucleotide primer
gggtctagagtaagtaaaattacgagcctctgcagc

SEQ ID NO: 30 - DNA, 38 bp, Artificial Sequence
  Oligonucleotide primer
ggggggagatctatgggagccatagattcattttgtgg

SEQ ID NO: 31 - DNA, 33 bp, Artificial Sequence
  Oligonucleotide primer
gggggatcccctttgtagatgaagatctacggc

SEQ ID NO: 32 - DNA, 35 bp, Artificial Sequence
  Oligonucleotide primer
gggggggtcgacttgtagcttgtaggctgtcgccg

SEQ ID NO: 33 - DNA, 41 bp, Artificial Sequence
  Oligonucleotide primer
gggtctagagcaatacaatcatagataacatcgttgagccc

SEQ ID NO: 34 - DNA, 35 bp, Artificial Sequence
  Oligonucleotide primer
gggggggaattcatggcaaccttctccgctactgg

SEQ ID NO: 35 - DNA, 32 bp, Artificial Sequence
  Oligonucleotide primer
gggggatcccagctgaggtatgggagccatag

SEQ ID NO: 36 - DNA, 36 bp, Artificial Sequence
  Oligonucleotide primer
ggggggtctagatcacacgggcccaatgactgaacc

SEQ ID NO: 37 - DNA, 32 bp, Artificial Sequence
  Oligonucleotide primer
ggggaattccatgcgcagtaggtcattgtagc

SEQ ID NO: 38 - DNA, 38 bp, Artificial Sequence
  Oligonucleotide primer
ccccttaagtgccatatacggctcaccaccatatacac

SEQ ID NO: 39 - DNA, 32 bp, Artificial Sequence
  Oligonucleotide primer
ggggggatcctggtacgttgggctcaacgatg

SEQ ID NO: 40 - DNA, 37 bp, Artificial Sequence
  Oligonucleotide primer
cccttccggaggttcctgggtggctaataactacttc

SEQ ID NO: 41 - DNA, 35 bp, Artificial Sequence
  Oligonucleotide primer
gggggtctagagtcaccacaaaatgaatctatggc

SEQ ID NO: 42 - DNA, 22 bp, Artificial Sequence
  Oligonucleotide primer
ggggggatcccagtactttggc

SEQ ID NO: 43 - DNA, 33 bp, Artificial Sequence
  Oligonucleotide primer
ggggggatcccgcagttggtaacaagcttgtcg

SEQ ID NO: 44 - DNA, 29 bp, Artificial Sequence
  Oligonucleotide primer
ggggggatcccagcgacacccgaggcacg

SEQ ID NO: 45 - DNA, 34 bp, Artificial Sequence
  Oligonucleotide primer
gggggtctagaccacacaggagaatatccacgtc

SEQ ID NO: 46 - DNA, 36 bp, Artificial Sequence
  Oligonucleotide primer
ggggggcatgctgtaggtgggccatggatcaagtcc

SEQ ID NO: 47 - DNA, 33 bp, Artificial Sequence
  Oligonucleotide primer
ggggtcgacgtcaccacaaaatgaatctatggc

SEQ ID NO: 48 - DNA, 33 bp, Artificial Sequence
  Oligonucleotide primer
ggggggcatgccacctgggccaagaaattgacc

SEQ ID NO: 49 - DNA, 31 bp, Artificial Sequence
  Oligonucleotide primer
ggggaattccacacaggagaatatccacgtc

SEQ ID NO: 50 - DNA, 24 bp, Artificial Sequence
  Oligonucleotide primer
gctggccgcgtaatgctggttggg

SEQ ID NO: 51 - DNA, 22 bp, Artificial Sequence
  Oligonucleotide primer
caactatgccgaattcacggcc

SEQ ID NO: 52 - DNA, 26 bp, Artificial Sequence
  Oligonucleotide primer
ccgtgctggatgtgaggccgttaccg

SEQ ID NO: 53 - DNA, 22 bp, Artificial Sequence
  Oligonucleotide primer
ggccgtgaattcggcatagttg

SEQ ID NO: 54 - DNA, 37 bp, Artificial Sequence
  Oligonucleotide primer
ggggggatccatgttatctatgattgtattgctattc

SEQ ID NO: 55 - DNA, 32 bp, Artificial Sequence
  Oligonucleotide primer
ggggggatccatttctgtgattgatgcagcgc

SEQ ID NO: 56 - DNA, 35 bp, Artificial Sequence
  Oligonucleotide primer
ggggggtctagacaacacaactatgccgaattcac

SEQ ID NO: 57 - DNA, 38 bp, Artificial Sequence
  Oligonucleotide primer
gggggtctagaggcatattcataacccgtgtgcactac

SEQ ID NO: 58 - DNA, 23 bp, Artificial Sequence
  Oligonucleotide primer
atggtatggctagcatgactggt

SEQ ID NO: 59 - DNA, 35 bp, Artificial Sequence
  Oligonucleotide primer
ggggggatcctttgaccggaccggccacatgggtc

SEQ ID NO: 60 - DNA, 23 bp, Artificial Sequence
  Oligonucleotide primer
caaacaacagatggctggcaact

SEQ ID NO: 61 - DNA, 38 bp, Artificial Sequence
  Oligonucleotide primer
gggggtctagagtaagtaaaattacgagcctctgcagc